(19) United States
(12) Patent Application Publication
(10) Pub. No.: US 2016/0282351 A1
(43) Pub. Date: Sep. 29, 2016

(54) SALIVARY BIOMARKERS FOR CANCERS, METHODS AND DEVICES FOR ASSAYING THE SAME, AND METHODS FOR DETERMINING SALIVARY BIOMARKERS FOR CANCERS

(71) Applicant: SALIVATECH CO., LTD., Tsuruoka-shi, Yamagata (JP)

(72) Inventors: Masahiro SUGIMOTO, Tsuruoka-sho (JP); Tomoyoshi SOGA, Tsuruoka-shi (JP); Makoto SUNAMURA, Tokyo (JP)

(73) Assignee: SALIVATECH CO., LTD., Tsuruoka-shi, Yamagata (JP)

(21) Appl. No.: 15/032,715

(22) PCT Filed: Oct. 28, 2014
(86) PCT No.: PCT/JP2014/078671

§ 371 Date: Apr. 28, 2016

(30) Foreign Application Priority Data
Oct. 28, 2013 (JP) 2013-223738

(51) Int. Cl. G01N 33/574

(52) U.S. Cl. CPC G01N 33/57488; G01N 33/57415; G01N 33/57438; G01N 33/57407

(57) ABSTRACT

Salivary biomarker characterized as low-molecular-weight compounds named metabolites or combinations of these biomarkers are used for detecting cancers. The salivary biomarker for cancer can be, for example, a combination of creatinine, N1-acetylspermidine, α-aminoadipic acid, N-acetylneuraminic acid, and 1,3-diaminopropane. Due to this configuration, the early detection of pancreatic cancer, breast cancer, oral cancer, and the like is possible in a healthy subject even with saliva having large concentration fluctuations.

### TECHNICAL FIELD

The present invention relates to salivary biomarkers for cancers, methods and devices for assaying the same, and methods for determining the salivary biomarkers for cancer. In particular, the present invention relates to salivary biomarkers to differentiate pancreatic cancer, intraductal papillary mucinous neoplasm (IPMN), breast cancer, and oral cancers from healthy controls, and methods and devices for assaying these biomarkers, and methods for determining these salivary biomarkers.

### BACKGROUND ART

A treatment of pancreatic cancer patients, one of the most maglicant cancers showing a poor prognosis, is still difficult. The median survival year is less than one year for pancreatic cancer patients who do not undego adjuvant therapies, such as chemotherapy and radiotherapy. Thus, detection of pancreatic cancer at the early stages is the only way available to prove the prognosis, indicating the needs of development of novel methods to detect the cancer using a biological sample (body fluid, etc.) minimally or non-invasively.

One of the present inventors previously proposed serum biomarkers to detect liver diseases in Patent Literatures 2 and 3.

Large molecule biomarkers for early detection of pancreatic cancers using blood, serum and plasma samples have been intensively developed (Patent Literatures 4 and 5). For example, carbohydrate antigen 19-9 (CA19-9) is already commonly used as a tumor marker to detect pancreatic cancers and biliary tract cancers as well as to evaluate the effects of chemotherapy. However, early detection of pancreatic cancer using this marker is difficult, and the accuracy of screening cancer is insufficient (Non-Patent Literature 1). In addition, CA19-9 levels do not increase in Lewis negative patients even in the advanced stage. Detection of a pancreatic cancer associated antigen (DUPAN-2 antigen) and a carcinoembryonic antigen (CEA) are also used. However, DUPAN-2 shows low specificity because this marker increases not only for pancreatic cancer but also for biliary tract and liver cancers. CEA also shows low specificity and shows positive for cancers of the digestive system, e.g. esophageal cancer and gastric cancer. Therefore, these markers are not specific to pancreatic cancer. Further, these two markers have not been widely used due to costs.

Polyamines, such as spermine (spermine), and acetylated polyamines, such as N8-acetylspermidine (N8-Acetylspermidine), N1-acetylspermidine (N1-Acetylspermidine), and N1-acetylspermine (N1-Acetylspermine) were known as metabolite biomarkers for various cancers in blood and urine (Non-Patent Literature 2). In a metabolic pathway, arginine is metabolized to ornithine, and then metabolized through putrescine to polyamines. The synthesis of polyamines is usually relatively activated in close to the surface of tumor tissues where oxygen is available compared to the center of the tumor tissue, while synthesis is less activated under a hypoxic condition in the center of the tumor. Despite their hetelogenious conditions in the tumor tissues, overall, the concentration of the polyamines in total tumor tissue increases and a part of these metabolites is transferred to the blood vessel. For example, an increase in the concentration of spermidine in blood is known in patients with breast cancers, prostate cancers and testis tumors (Non-Patent Literature 1). Decreasing the concentrations of spermine and spermidine in blood is reported in patients with acute pancreatitis by experiments on animals (Non-Patent Literature 3).

### CITATION LIST

### Patent Literature

Patent Literature 1: Japanese Patent Application Laid-Open No. 2011-58863
Patent Literature 2: Japanese Patent Application Laid-Open No. 2011-232164
Patent Literature 3: WO2011/158590A1
Patent Literature 4: Japanese Patent Application Laid-Open No. 2011-247869
Patent Literature 5: Japanese Translation of PCT International Application No. 2009-508493
Patent Literature 6: Japanese Patent Application Laid-Open No. 2013-521763

### Non-Patent Literature

Non-Patent Literature 1: Hamada S, Shimosegawa T., Biomarkers of pancreatic cancer, Pancreatology. 2011; 11: 14-9
Non-Patent Literature 2: Soda K (2011), The mechanisms by which polyamines accelerate tumor spread. Journal of Experimental & Clinical Cancer Research. 30(1): 95
Non-Patent Literature 3: Jin H T, Lamsa T, Merentie M, Hyvonen M T, Sand J, Raty S, Herzig K H, Alhonen L, Nordback I (2008). Polyamine levels in the pancreas and the blood change according to the severity of pancreatitis. Pancreatology. 8(1), 15-24
Non-Patent Literature 4: Zhang L, Farrell J J, Zhou H, Elashoff D, Akin D, Park N H, Chia D, Wong D T. (2010), Salivary transcriptomic biomarkers for detection of resectable pancreatic cancer. Gastroenterology. 138(3): 949-57
Non-Patent Literature 5: Sugimoto M, Wong D T, Hirayama A, Soga T, Tomita M, (2010), Capillary electrophoresis mass spectrometry-based saliva metabolomics identified oral, breast and pancreatic cancer-specific profiles, Metabolomics, 6, 78-95
Non-Patent Literature 6: Soga, T., Baran, R., Suematsu M., Ueno, Y., Ikeda, S., Sakurakawa T., Kakazu, Y., Ishikawa, T., Robert, M., Nishioka, T., Tomita, M. (2006), Differential methabolomics reveals ophthalmic acid as an oxidative stress biomarker indicating hepatic glutathione sonsumption., Journal of Biological Chemistry, 281 (24): 16768-16776
Non-Patent Literature 7: Sugimoto et al. Capillary electrophoresis mass spectrometry-based saliva metabolomics identified oral, breast and pancreatic cancer-specific profiles, Metabolomics, 2010, 6, 78-95
Non-Patent Literature 8: Tsutsui et al, High-throughput LC-MS/MS based simultaneous determination of polyamines including N-acetylated forms in human saliva and the diagnostic approach to breast cancer patients, Anal Chem, 2013, 85, 11835-42
Non-Patent Literature 9: Wang Q et al. Investigation and identification of potential biomarkers in human saliva for the early diagnosis of oral squamous cell carcinoma., Clin Chim Acta. 2014; 427: 79-85

### SUMMARY OF INVENTION

### Technical Problem

Conventional screening for protein markers in blood, serum, or plasma is insufficient for early detection of pancreatic cancer. Although blood-based tests are minimally invasive, professionals, such as medical doctors and nurses are required to handle the syringe. Thus, frequency of the test is limited. In contrast, the use of saliva provides definite advantages, i.e. completely non-invasive collection anywhere, which make it possible for frequent and self-sampling tests. For example, salivary biomarkers for lung cancer detection was proposed in Patent Literature 6. As mentioned, because early detection of pancreatic cancer using currently known biomarkers is difficult, frequent salivary testing is the only method to increase the possibility of detecting this cancer in earlier stages.

Detection of pancreatic cancer using mRNA profiles in saliva was proposed in Non-Patent Literature 4.

However, quantification of mRNA requires complex sample processing and addition of RNase inhibitor to saliva just after saliva collection to prevent mRNA degradation. Because of the low reproducibility of microarray-based quantification of mRNA, quantitative PCR (qPCR) is usually used for validating a marker's quantified values. However, each qPCR can profile only one marker, which limits simultaneous quantification of multiple markers. For example, only 35 substances are quantitatively determined in Non-Patent Literature 4. Thus, the use of qPCR limits simultaneous quantification of multiple markers, which cannot capture the holistic view of salivary molecular characteristics, e.g. the overall variation of salivary concentration cannot be determined. Therefore, highly accurate prediction using only a few markers becomes difficult. In the case of qPCR-based quantification, complexity of the sample processing for qPCR may increase artificial noise levels. Thefore, simple methods for quantifying salivary moleculers to minimize possible artificial noise are preferable. Taken together, not only exploring novel biomarkers but also the development of combination techniques for accurately detecting subjects with various cancers, such as pancreatic, breast and oral cancers, and begin diseases including intraductal papillary mucinous neoplasm (IPMN), are required.

The present invention addresses these problems. An object of the present invention is the early detection of cancer such as pancreatic cancer, breast cancer, and oral cancer using saliva.

### Solution to the Problems

The present inventors identified multiple metabolite biomarkers in saliva to discriminate patients with pancreatic cancers from healthy controls. Capillary electrophoresis-mass spectrometry (CE-MS) may be used to simultaneously quantify these metabolite markers. The inventors also developed combinations of these biomarkers to realize accurate discrimination. Although saliva samples should be collected carefully to eliminate diurnal variation, there are difficulties to completely eliminate these variations. Therefore, the inventors also found normalization metabolites for estimating the total concentration of the metabolites in saliva, and developed algorithms to combine metabolite markers and normalization metabolites for more accurate detection of subjects with pancreatic cancers.

Further, the inventors have found markers for breast cancer and oral cancer following similar procedures.

The present invention is based on the aforementioned research results. Salivary biomarkers and their combinations have the potential to solve the aforementioned problems.

Herein, salivary metabolite biomarkers and their combinations were developed to detect certain diseases, including pancreatic cancer, intraductal papillary mucinous neoplasm (IPMN), breast cancer, and oral cancer.

Absolute concentration and the combination of the following salivary metabolite biomarkers can be used for detecting patients with pancreatic disease: N-acetylputrescine (N-Acetylputrescine), adenosine (Adenosine), 3-phospho-D-glyceric acid (3PG), urea (Urea), o-acetylcarnitine (o-Acetylcarnitine), citric acid (Citrate), glycyl-glycine (Gly-Gly), 5-aminovaleric acid (5-Aminovalerate), 4-methyl 2-oxopentanoate (2-Oxoisopentanoate), malic acid (Malate), benzoate ester (Benzoate), fumaric acid (Fumarate), N-acetylaspartic acid (N-Acetylaspartate), inosine (Inosine), 3-methylhistidine (3-Methylhistidine), N1-acetylspermine (N1-Acetylspermine), creatine (Creatine), α-aminoadipic acid (alpha-Aminoadipate), phosphorylcholine (Phosphorylcholine), 2-hydroxypentanoate (2-Hydroxypentanoate), xanthine (Xanthine), succinic acid (Succinate), 6-phosphogluconic acid (6-Phosphogluconate), butanoic acid (Butanoate), homovanillic acid (Homovanillate), O-phosphoserine (O-Phosphoserine), trimethylamine-N-oxide (Trimethylamine N-oxide), piperidine (Piperidine), cystine (Cys), 2-isopropylmalic acid (2-Isopropylmalate), N8-acetylspermidine (N8-Acetylspermidine), N1-acetylspermidine (N1-Acetylspermidine), N-acetylneuraminic acid (N-Acetylneuraminate), glucosamine (Glucosamine), spermine (Spermine), agmatine (Agmatine), N-acetylhistamine (N-Acetylhistamine), methionine (Met), p-4-hydroxyphenylacetic acid (p-4-Hydroxyphenylacetate), N,N-dimethylglycine (N,N-Dimethylglycine), hypotaurine (Hypotaurine), glutamyl-glutamic acid (Glu-Glu), and N1,N12-diacetylspermine (N1,N12-Diacetylspermine).

Relative concentration, i.e. the absolute concentration divided by the concentration of the normalization metabolite, of the following salivary metabolite biomarkers can be used for detecting patients with pancreatic cancer or IPMN: N8-acetylspermidine (N8-Acetylspermidine), creatinine (Creatinine), spermine (Spermine), aspartic acid (Asp), N1-acetylspermidine (N1-Acetylspermidine), N1-acetylspermine (N1-Acetylspermine), cytidine (Cytidine), α-aminoadipic acid (alpha-Aminoadipate), cytosine (Cytosine), betaine (Betaine), urea (Urea), homovanillic acid (Homovanillate), N-acetylneuraminic acid (N-Acetylneuraminate), cystine (Cys), urocanic acid (Urocanate), fumaric acid (Fumarate), 1,3-diaminopropane (1,3-Diaminopropane), hypotaurine (Hypotaurine), nicotinic acid (Nicotinate), agmatine (Agmatine), valine (Val), 2-hydroxy-4-methylpentanoic acid (2-Hydroxy-4-methylpentanoate), alanyl-alanine (Ala-Ala), citric acid (Citrate), glucosamine (Glucosamine), carnosine (Carnosine), glycyl-glycine (Gly-Gly), 2-aminobutyric acid (2AB), arginine (Arg), N-acylglutamic acid (N-Acetylglutamate), glycerophosphoric acid (Glycerophosphate), phosphoenolpyruvic acid (PEP), isoleucine (Ile), adenosine (Adenosine), guanine (Guanine), dihydroxyacetonephosphoric acid (DHAP), and cadaverine (Cadaverine).

As an example combination, the absolute concentration of creatinine, N1-acetylspermidine, α-aminoadipic acid, N-acetylneuraminic acid, and 1,3-diaminopropane in saliva can be used for accurate pancreatic cancer detection. The prediction can be made by using another combination or changing the methodology of combination.

As the salivary biomarker for cancer used to detect breast cancer, the absolute concentration of the following substances or a combination thereof in saliva can be used: choline (Choline), 2-hydroxybutyric acid (2-Hydroxybutyrate), β-alanine (beta-Ala), 3-methylhisdine (3-Methylhistidine), α-aminobutyric acid (2AB), N-acetyl-β-alanine (N-Acetyl-beta-alanine), isethionic acid (Isethionate), N-acetylphenylalanine (N-Acetylphenylalanine), trimethyllysine (N6,N6,N6-Trimethyllysine), α-aminoadipic acid (alpha-Aminoadipate), creatine (Creatine), γ-butyrobetaine (gamma-Butyrobetaine), sarcosine (Sarcosine), pyruvic acid (Pyruvate), urocanic acid (Urocanate), piperidine (Piperidine), serine (Ser), homovanillic acid (Homovanillate), 5-oxoproline (5-Oxoproline), GABA (GABA), 5-aminovaleric acid (5-Aminovalerate), trimethylamine-N-oxide (Trimethylamine N-oxide), 2-hydroxyvaleric acid (2-Hydroxyl)pentanoate), carnitine (Carnitine), isopropanolamine (Isopropanolamine), hypotaurine (Hypotaurine), lactic acid (Lactate), 2-hydroxy-4-methylpentanoic acid (2-Hydroxy-4-methylpentanoate), hydroxyproline (Hydroxyproline), butyric acid (Butanoate), adenine (Adenine), N6-acetyllysine (N-epsilon-Acetyllysine), 6-hydroxyhexanoic acid (6-Hydroxyhexanoate), propionic acid (Propionate), betaine (Betaine), N-acetylputrescine (N-Acetylputrescine), hypoxanthine (hypoxanthine), crotonic acid (Crotonate), tryptophan (Trp), citrulline (Citrulline), glutamine (Gln), proline (Pro), 2-oxoisopentanoic acid (2-Oxoisopentanoate), 4-methylbenzoate (4-Methylbenzoate), 3-(4-hydroxyphenyl)propionic acid (3-(4-Hydroxyphenyl)propionate), cysteic acid (Cysteate), azelaic acid (Azelate), ribulose-5-phosphoric acid (Ru5P), pipecolinic acid (Pipecolate), phenylalanine (Phe), O-phosphoserine (O-Phosphoserine), malonic acid (Malonate), hexanoic acid (Hexanoate), and p-hydroxyphenylacetic acid (p-Hydroxyphenylacetate).

The aforementioned substances that are significant substances and that are not publicly known are indicated in Table 7 below.

A combination of β-alanine, N-acetylphenylalanine, and citrulline can be used as one example of a combination of salivary biomarkers for cancer to detect breast cancer. The prediction can be performed by using a different combination or changing the methodology of combination.

When a value is used in which the concentration of saliva was corrected, the following substances or a combination thereof may be used as a marker: choline (Choline), β-alanine (beta-Ala), 3-methylhisdine (3-Methylhistidine), α-aminobutyric acid (2AB), N-acetyl-β-alanine (N-Acetyl-beta-alanine), isethionic acid (Isethionate), N-acetylphenylalanine (N-Acetylphenylalanine), trimethyllysine (N6,N6,N6-Trimethyllysine), urocanic acid (Urocanate), piperidine (Piperidine), 5-aminovaleric acid (5-Aminovalerate), trimethylamine-N-oxide (Trimethylamine N-oxide), isopropanolamine (Isopropanolamine), hypotaurine (Hypotaurine), hydroxyproline (Hydroxyproline), N6-acetyllysine (N-epsilon-Acetyllysine), 6-hydroxyhexanoic acid (6-Hydroxyhexanoate), N-acetylputrescine (N-Acetylputrescine), azelaic acid (Azelate), dihydroxyacetonephosphoric acid (DHAP), glycolic acid (Glycolate), 4-methyl-2-oxopentanoic acid (4-Methyl-2-oxopentanoate), N-acetylaspartic acid (N-Acetylaspartate), glycerophosphoric acid (Glycerophosphate), 3-hydroxybutyric acid (3-Hydroxybutyrate), benzoic acid (Benzoate), adipic acid(Adipate), 2-isopropylmalate (2-Isopropylmalate), phosphorylchlorine (Phosphorylcholine), N-acetylneuraminic acid (N-Acetylneuraminate), histamine (His), o-acetylcarnitine (o-Acetylcarnitine), N-acetylglucosamine 1-phosphate (N-Acetylglucosamine 1-phosphate), creatinine (Creatinine), arginine (Arg), and syringic acid (Syringate).

The aforementioned substances that are significant substances and that are not publicly known are indicated in Table 8 below.

A combination of N-acetylphenylalanine, N-acetylspermidine, and creatine can be used as one example of a combination of salivary biomarkers for cancer used to detect breast cancer. The prediction can be performed by using a different combination or changing the methodology of combination.

As the salivary biomarker for cancer used to detect oral cancer, the concentration of the following substances or a combination thereof in saliva can be used: Glycyl-glycine (Gly-Gly), citrulline (Citrulline), γ-butyrobetaine (gamma-Butyrobetaine), 3-phenyllactate (3-Phenyllactate), butyric acid (Butanoate), hexanoic acid (Hexanoate), methionine (Met), hypoxanthine (Hypoxanthine), spermidine (Spermidine), tryptophan (Trp), aspartic acid (Asp), isopropanolamine (Isopropanolamine), alanyl-alanine (Ala-Ala), N,N-dimethylglycine (N,N-Dimethylglycine), N1-acetylspermidine (N1-Acetylspermidine), N1-,N8-diacetylspermidine (N1,N8-Diacetylspermidine), N8-acetylspermidine (N8-Acetylspermidine), α-aminobutyric acid (2AB), trimethylamine-N-oxide (Trimethylamine N-oxide), N-acetylaspartic acid (N-Acetylaspartate), adenine (Adenine), 2-hydroxyvaleric acid (2-Hydroxyl)pentanoate), putrescine (Putrescine (1,4-Butanediamine)), 3-phosphoglycerate (3PG), 3-phenylpropionic acid (3-Phenylpropionate), serine (Ser), 1-methylnicotinamide (1-Methylnicotineamide), 3-hydroxy-3-methylglutaric acid (3-Hydroxy-3-methylglutarate), guanine (guanine), 3-(4-hydroxyphenyl)propionic acid (3-(4-Hydroxyphenyl)propionate), 4-methylbenzoate (4-Methylbenzoate), ribulose-5-phosphoric acid (Ru5P), α-aminoadipic acid (alpha-Aminoadipate), N6-acetyllysine (N-epsilon-Acetyllysine), glucosamine (Glucosamine), cystine (Cys), carnosine (Carnosine), urocanic acid (Urocanate), phenylalanine (Phe), 2-deoxyribose-1-phosphoric acid (2-Deoxyribose 1-phosphate), cytidine disodium 5′-monophosphate (CMP), p-hydroxyphenylacetic acid (p-Hydroxyphenylacetate), 3-hydroxybutyric acid (3-Hydroxybutyrate), N-acetylputrescine (N-Acetylputrescine), 7-methylguanine (7-Methylguanine), inosine (Inosine), lysine (Lys), dihydroxyacetonephosphoric acid (DHAP), 3-methylhisdine (3-Methylhistidine), carbamoylaspartic acid (Carbamoylaspartate), creatinine (Creatinine), N-methyl-2-pyrrolidone (1-Methyl-2-pyrrolidinone), pyruvic acid (Pyruvate), propionic acid (Propionate), 5-aminovaleric acid (5-Aminovalerate), N-acetylornithine (o-Acetylornithine), 5-oxoproline (5-Oxoproline), creatine (Creatine), homoserine (Homoserine), fumaric acid (Fumarate), glycine (Gly), and N1,N12-diacetylspermine (N1,N12-Diacetylspermine).

The aforementioned substances that are not publicly known are indicated in Table 9 below.

The present invention provides a method for assaying a salivary biomarker for cancer including the steps of: collecting a saliva sample; and detecting the aforementioned salivary biomarker for cancer in the collected saliva sample.

The present invention provides a device for assaying a salivary biomarker for cancer including means for collecting a saliva sample, and means for detecting the aforementioned salivary biomarker for cancer in the collected saliva sample.

The present invention further provides a method for determining a salivary biomarker for cancer including a procedure of performing ultrafiltration of a saliva sample, means for cyclopedically measuring ionic metabolites in the saliva sample after the ultrafiltration, and a procedure of selecting a substance having high ability of distinguishing a patient with a pancreatic disease from a healthy subject according to concentrations of the measured metabolites.

Correlation of absolute concentration among multiple metabolites can be used for identifying a normalizing metabolite that can eliminate variation of overall concentrations in saliva.

A combination of the salivary biomarkers for cancer can be determined using a mathematical model.

### Advantageous Effects of Invention

According to the present invention, not only pancreatic cancer but also a pancreatic disease including IPMN and chronic pancreatitis, breast cancer, and oral cancer can be detected early using saliva that can be collected non-invasively and simply. In particular, a combination of polyamine with novel metabolite biomarkers makes a highly accurate prediction possible.

### BRIEF DESCRIPTION OF THE DRAWINGS

FIG. 1 is a flowchart illustrating the procedure for determining the biomarkers used in the Examples of the present invention.

FIG. 2 is a diagram illustrating a correlation network between metabolites in saliva used in the Examples.

FIG. 3 is a flowchart illustrating a procedure of developing a mathematical model used in the Examples.

FIG. 4 is a diagram illustrating a model of a decision tree that distinguishes a subject with pancreatic cancer from a healthy subject.

FIG. 5 is a diagram illustrating a receiver operating characteristic (ROC) curve of a mathematical model that distinguishes a subject with pancreatic cancer from a healthy subject using a metabolite concentration normalized with a concentration marker used in the Examples.

FIG. 6 is a diagram in which a risk of pancreatic cancer (PC) for a healthy subject (C), and subjects with pancreatic cancer (PC), chronic pancreatitis (CP), and IPMN is plotted in a model of classifying the healthy subject and the subject with pancreatic cancer in the Examples.

FIG. 7 is a diagram illustrating a stepwise forward selection method used for variable selection in an MLR model that distinguishes a subject with pancreatic cancer from a healthy subject when the absolute concentration of the concentration marker as used in the Examples.

FIG. 8 is a diagram illustrating a forward selection method used for variable selection in the MLR model that distinguishes a subject with pancreatic cancer from a healthy subject when the absolute concentration of the concentration marker as used in the Examples.

FIG. 9 is a diagram illustrating an example of a total concentration of amino acids in saliva used in the Examples.

FIG. 10 is a diagram illustrating an ROC curve in which variables in an MLR model that distinguishes a patient with breast cancer from a healthy subject are β-alanine, N-acetylphenylalanine, and citrulline.

FIG. 11 is a diagram illustrating a ROC curve in which the variables in the same MLR model are N-acetylphenylalanine, N1-acetylspermidine, and creatine.

FIG. 12 is a diagram of a network between metabolites for determination of a concentration-correcting substance for a biomarker for breast cancer.

FIG. 13 includes diagrams illustrating substances belonging to polyamines among substances that give a significant difference between the healthy subject and the patient with breast cancer.

FIG. 14 includes diagrams illustrating examples of substances other than polyamines among the substances that give a significant difference between the healthy subject and the patient with breast cancer.

FIG. 15 includes diagrams illustrating the top five substances that give a significant difference between the healthy subject and the patient with breast cancer and has a smaller p value regardless of the presence or absence of concentration correction, and an ROC curve thereof.

FIG. 16 is a correlation network diagram illustrating a reason for determining Gly to be a correction marker.

FIG. 17 includes diagrams illustrating the concentrations of metabolites in a cancer tissue sample obtained during surgery of oral cancer and a healthy tissue sample near the cancer tissue sample.

FIG. 18 includes diagrams illustrating a difference in the concentration of saliva of a patient with oral cancer from that of the healthy subject when a method of collecting saliva in the patient is changed.

### DESCRIPTION OF EMBODIMENTS

Hereinafter, an embodiment suitably implementing the present invention (hereinafter referred to as the embodiment) will be described in detail. The present invention is not limited to the following embodiments and Examples. In addition, constituents in the following embodiments and Examples include those that can be easily assumed by those skilled in the art, those that are substantially equivalent, and those falling within the scope of the so-called doctrine of equivalents. Further, the constituents disclosed in the following embodiments and Examples may be used in appropriate combination or by appropriate selection.

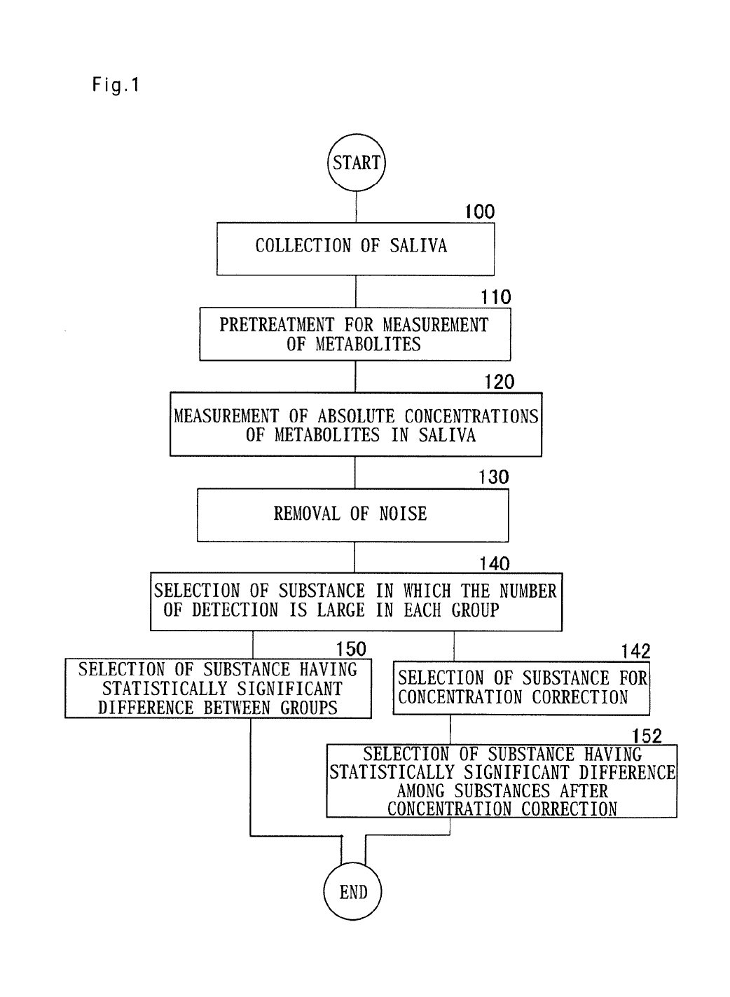

A procedure of determining a biomarker for a pancreatic disease will be described with reference to FIG. 1.

### 1. Saliva Donor

A total of 199 salivary samples were collected from patients with pancreatic cancer with various stages, healthy subjects, and patients with intraductal papillary mucinous neoplasm (IPMN) and chronic pancreatitis. Table 1 lists subject characteristics, such as sex and age. Of these, no patients had undergone chemotherapy.

TABLE 1

| | | THE | SEX | | | AGE | | | |
|---|---|---|---|---|---|---|---|---|---|
| | | NUMBER | | | THE NUMBER | | | | THE NUMBER |
| DISEASE | STAGE | OF CASES | male | female | OF DEFECTS | MINIMUM | MEDIAN | MAXIMUM | OF DEFECTS |
| HEALTHY | — | 63 | 50 | 13 | | 19 | 43 | 73 | |
| CHRONIC | — | 14 | 12 | 2 | | 32 | 49 | 79 | |
| PANCREATITIS | | | | | | | | | |
| TPMN | — | 7 | 6 | 1 | | 63 | 64 | 69 | 2 |
| PANCREATIC | I | 3 | 1 | 2 | | 61 | 68 | 76 | |
| CANCER | II | 3 | 1 | 1 | 1 | 67 | 67.5 | 68 | 1 |
| | III | 18 | 10 | 8 | | 51 | 69.5 | 95 | |
| | Iva | 37 | 14 | 16 | 7 | 45 | 69 | 85 | 7 |
| | Ivb | 54 | 30 | 22 | 2 | 43 | 72 | 86 | 3 |
| | SUBTOTAL OF | 115 | 56 | 49 | 10 | 43 | 70 | 95 | 11 |
| | PANCREATIC | | | | | | | | |
| | CANCER | | | | | | | | |

### 2. Method for Collecting Saliva (Step 100 in FIG. 1)

With respect to collection date

Collection is performed on a day other than a surgery day as much as possible.

With respect to diet

After 21:00 of the day before the collection, do not drink anything but water.

On the day of collection, do not eat breakfast.

Notes before collection of saliva on the day

Collect saliva from AM 8:30 to 11:00 before breakfast.

Brush teeth without use of toothpaste 1 hour or more before the collection of saliva.

Do not strenuously exercise 1 hour before the collection of saliva.

Do not clean the inside of oral cavity (with a toothpick, etc.).

Do not smoke.

Do not drink anything but water.

Method for collecting saliva

The mouth is rinsed with water before the collection of saliva, and non-irritant mixed saliva is collected.

Only saliva that runs spontaneously but is not volitionally generated is collected (sialemesis method). Alternatively, a straw is placed in the mouth when saliva is retained to some extent in the mouth (the time is about 3 minutes), and the saliva runs into a tube (passive drool method). When the face is turned down and saliva in the mouth is pushed into the straw that is vertically set, the saliva is likely to run spontaneously. However, when the saliva adheres to a middle of the straw and does not fall down, the saliva is sent out by the breath (in this case, saliva is easily collected by retaining saliva in the mouth to some extent and then pushing the saliva into the tube at one time as compared with opening of the mouth to the tube).

200 μL or more of saliva (as much as possible) is collected.

During the collection of saliva, the tube is placed on ice and kept at a low temperature as much as possible, and the collection is finished within 15 minutes (even when 200 μL of saliva is not collected, the collection is finished in 15 minutes).

Within 5 minutes, the saliva is cryopreserved on ice at −80° C. or with dry ice for storage. The tube and the straw of collecting saliva is a tube and a straw made of a polypropylene material.

A method for collecting saliva is not limited to the aforementioned method, and another method may be used.

### 3. Pretreatment Method for Measurement of Metabolites in Saliva (Step 110 in FIG. 1)

400 μL of saliva sample is taken, placed in an ultrafiltration filter (molecular weight cutoff: 5,000 Da), and centrifuged at 4° C. and 9,100 g for 3.5 hours. 45 μL of the filtrate and 5 μL of an aqueous solution in which the concentration of each of methionine sulfone (Methionine sulfone), 2-morpholinoethanesulfonic acid (2-Morpholinoethanesulfonic acid), CSA (D-Camphor-10-sulfonic acid), 3-aminopyrrolidine (3-Aminopyrrolidine), and trimesic acid (Trimesate) is 2 mM are mixed to prepare 50 μL of a sample. Measurement was performed by the following method.

### 4. Measurement of Absolute Concentrations of Metabolites in Saliva by Capillary Electrophoresis-Time-of-Flight Mass Spectrometry (CE-TOFMS) (Step 120 in FIG. 1)

Ionic metabolites were identified and quantitatively determined from saliva by metabolome analysis using CE-MS.

A measurement method was performed in accordance with the method described in Non-Patent Literature 6. Hereinafter, the parameters will be described.

### 1) Cationic Metabolite Measurement Mode

HPCE

Capillary: fused silica, 50 μm in inner diameter×100 cm in length
Buffer: 1 M formic acid (formate)
Voltage: positive, 30 kV

### Temperature: 20° C.

Injection: injection under a pressure of 50 mbar for 5 seconds (about 3 nL)
Washing before measurement: with 30 mM ammonium formate (Ammonium Formate) at a pH of 9.0 for 5 minutes, ultrapure water for 5 minutes, and buffer for 5 minutes

TOFMS

Polarity: positive
Capillary voltage: 4,000 V
Fragmentor voltage: 75 V
Skimmer voltage: 50 V

### OCT RFV: 125 V

Drying gas: nitrogen (N₂), 10 L/min
Drying gas temperature: 300° C.
Nebulizer gas pressure: 7 psig
Sheath liquid: 50% methanol/0.1 μM Hexakis (2,2-difluoroethoxy) phosphazene-containing water
Flow rate: 10 mL/min
Reference m/z: 2 methanol ¹³C isotope [M+H]+m/z 66.063061,
Hexakis(2,2-difluoroethoxy)phosphazene [M+H]+m/z 622.028963

### 2) Anionic Metabolite Measurement Mode

HPCE

Capillary: COSMO (+), 50 μm in inner diameter×10.6 cm in length

Buffer: 50 mM ammonium acetate, pH: 8.5

Voltage: negative, 30 kV

Temperature: 20° C.

Injection: injection under a pressure of 50 mbar for 30 seconds (about 30 nL)

Washing before measurement: with 50 mM ammonium acetate at a pH of 3.4 for 2 minutes, and 50 mM ammonium acetate at a pH of 8.5 for 5 minutes

TOFMS

Polarity: negative
Capillary voltage: 3,500 V
Fragmentor voltage: 100 V
Skimmer voltage: 50 V

### OCT RFV: 200 V

Drying gas: nitrogen (N₂), 10 L/min
Drying gas temperature: 300° C.
Nebulizer gas pressure: 7 psig
Sheath liquid: 5 mM ammonium acetate and 50% methanol/0.1 μM Hexakis (2,2-difluoroethoxy) phosphazene-containing water
Flow rate: 10 mL/min
Reference m/z: 2 acetic acid ¹³C isotope [M−H]−m/z 120.038339, Hexakis(2,2-difluoroethoxy) phosphazene+acetic acid [M−H]− 680.035541
ESI needle: platinum

The anionic metabolite measurement may be performed before the cationic metabolite measurement.

### 5. Removal of Noise (Step 130 in FIG. 1)

Signals of a substance in which a value largely varies depending on a measurement day and a substance not derived from a metabolite are removed.

From measurement data, all peaks in which a signal noise ratio was 1.5 or more were first detected. A commercially available standard substance was measured before measurement of the saliva samples. A peak in which a value of mass to charge ratio (m/z) obtained by a mass spectrometer and a corresponding migration time were assigned to a substance name. Thus, identification was performed. In quantitative determination, the peak area of each peak was divided by the area of the peak of the internal standard substance, a fluctation of measurement sensitivity of the mass spectrometer was corrected, and the specific peak area ratio was calculated. The absolute concentration was calculated from a ratio of the specific peak area in the saliva samples to the specific peak area of the standard substance.

### 6. Selection of Substance Detected Highly Frequently in Each Group (Step 140 in FIG. 1)

Only a substance in which the peak can be detected in 30% or more cases (for example, three out of ten) of each group was selected.

### 7. Selection of a Substance Having a Statistically Significant Difference Between Groups (Step 150 in FIG. 1)

After a typical test (in this case, Mann-Whitney test) was performed, a P value was corrected using a false discovery rate (FDR) and a Q value was calculated. A substance having a significant difference of Q<0.05 was selected.

The substance selected by this procedure is a substance selected from N-acetylputrescine (N-Acetylputrescine), adenosine (Adenosine), 3-phospho-D-glyceric acid (3PG), urea (Urea), o-acetylcarnitine (o-Acetylcarnitine), citric acid (Citrate), glycyl-glycine (Gly-Gly), 5-aminovaleric acid (5-Aminovalerate), methyl 2-oxopentanoate (2-Oxoisopentanoate), malic acid (Malate), benzoate ester (Benzoate), fumaric acid (Fumarate), N-acetylaspartic acid (N-Acetylaspartate), inosine (Inosine), 3-methylhistidine (3-Methylhistidine), N1-acetylspermine (N1-Acetylspermine), creatine (Creatine), α-aminoadipic acid (alpha-Aminoadipate), phosphorylcholine (Phosphorylcholine), 2-hydroxypentanoate (2-Hydroxypentanoate), xanthine (Xanthine), succinic acid (Succinate), 6-phosphogluconic acid (6-Phosphogluconate), butanoic acid (Butanoate), homovanillic acid (Homovanillate), O-phosphoserine (O-Phosphoserine), trimethylamine-N-oxide (Trimethylamine N-oxide), piperidine (Piperidine), cystine (Cys), 2-isopropylmalic acid (2-Isopropylmalate), N8-acetylspermidine (N8-Acetylspermidine), N1-acetylspermidine (N1-Acetylspermidine), N-acetylneuraminic acid (N-Acetylneuraminate), glucosamine (Glucosamine), spermine (Spermine), agmatine (Agmatine), N-acetylhistamine (N-Acetylhistamine), methionine (Met), p-4-hydroxyphenylacetic acid (p-4-Hydroxyphenylacetate), N,N-dimethylglycine (N,N-Dimethylglycine), hypotaurine (Hypotaurine), glutamyl-glutamic acid (Glu-Glu), N1,N12-diacetylspermine (N1,N12-Diacetylspermine), and combinations thereof.

### 8. Selection of Substance for Presuming Concentrations of all Metabolites in Saliva and Performing Concentration Correction (Step 142 in FIG. 1)

In all the samples measured (including healthy, breast cancer, oral cancer, IPMN, and pancreatic cancer), correlation values between the metabolites were exhaustively calculated using the determined quantitative values of the metabolites. Combinations of substances satisfying a Pearson correlation coefficient (R) of R≧0.8 were listed. Of a metabolite group in which the most substances correlated with each other, a substance that correlated with the most substances was selected.

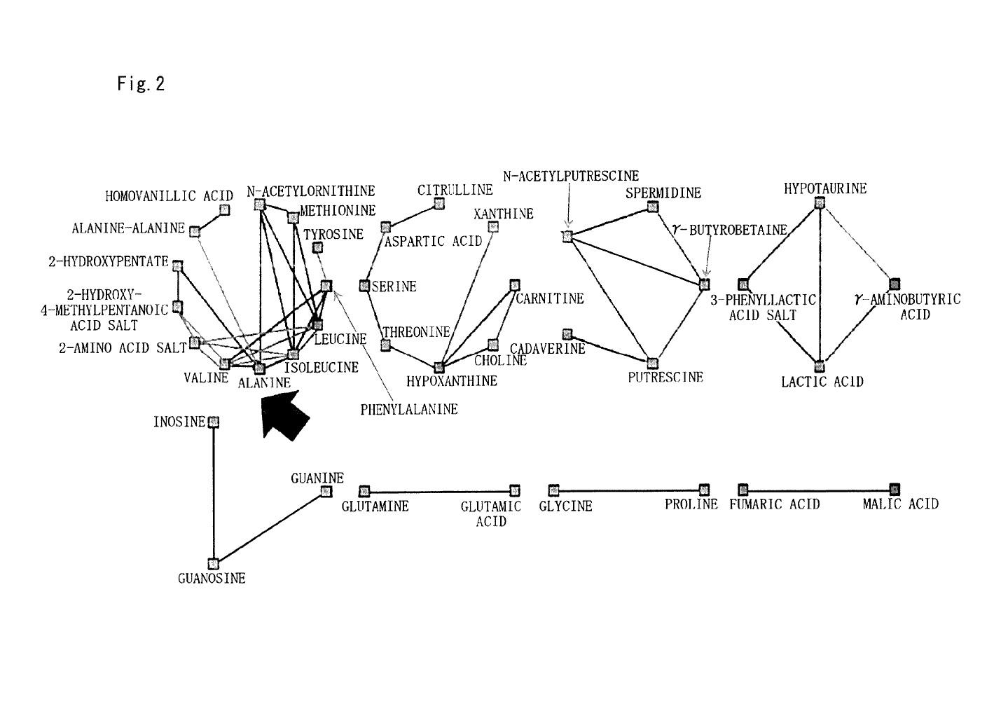

FIG. 2 shows one example of a correlation network diagram of the metabolites in saliva.

9. Selection of a Substance Having a Statistically Significant Difference Among the Substances after Concentration Correction (Step 152 in FIG. 1)

After the typical test (in this case, Mann-Whitney test) was performed using a value in which the concentration of each substance was corrected with the concentration of the substance selected at Step 142, a P value was corrected using the false discovery rate (FDR), and a Q value was calculated. A substance having a significant difference of Q<0.05 was selected.

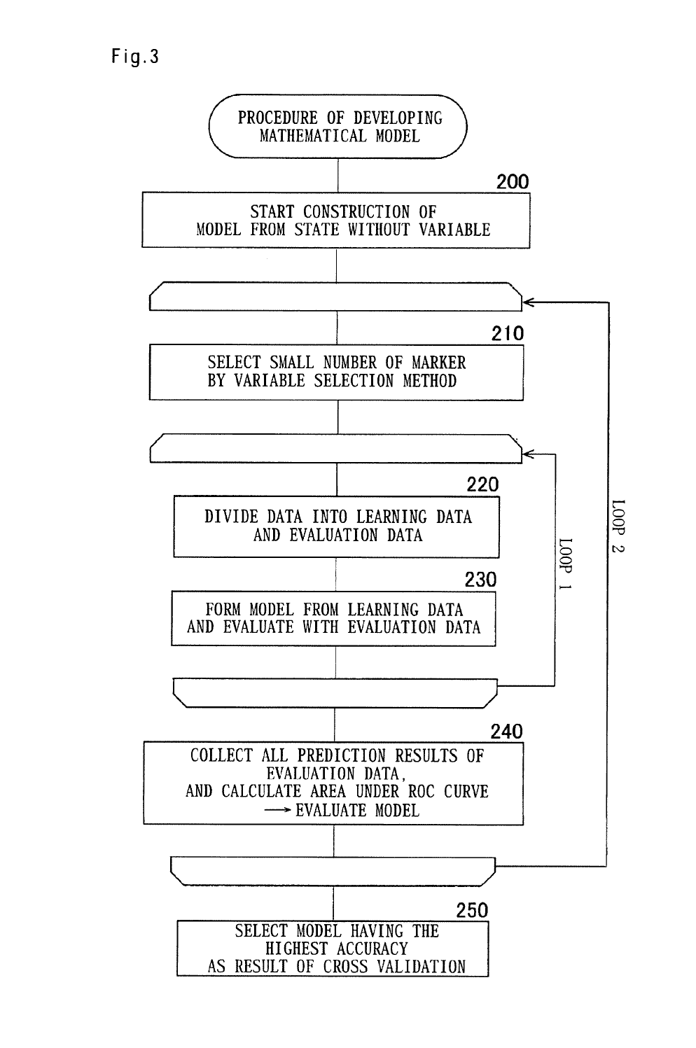

A procedure of developing a mathematical model of distinguishing the subjects with pancreatic cancer from the healthy subjects will be then described with reference to FIG. 3.

Using the marker selected at Step 150 or 152 in FIG. 1, a multiple logistic regression model (MLR model) that is a mathematical model was developed from a state in which a variable did not exist at Step 200. In the analysis of multiple logistic regression (MLR), a regression equation of P that is

ln(P/1−P)=b₀+b₁x₁+b₂x₂+b₃x₃+ . . . +bₖxₖ (1)

is determined using k description variables x₁, x₂, x₃, . . . , and xₖ for a ratio P as a target variable.

Specifically, a combination of the smallest independent variables that did not correlate with each other was selected at Step 210, for example, using a stepwise forward selection method of stepwise variable selection. A P value at which the variable was added was 0.05, a P value at which the variable was eliminated was 0.05, and a variable xᵢ was selected.

At Step 220, the data were divided into learning data and evaluation data, and at Step 230, a model was formed from the learning data and evaluated using the evaluation data. In cross validation of Loop 1 in FIG. 3, Steps 220 and 230 were repeated.

At Step 240, receiver operating characteristic (ROC) analysis was performed using the selected model. An area under the ROC curve (AUC) and a 95% confidential interval (CI) were calculated, and the model was evaluated. In accordance with the ROC curve, a curve of Y=X+α (α is a constant) was drawn. When the value of α was decreased from 1 to 0, the value of α that first touched the ROC curve was determined. Thus, an optimal cut-off value was determined.

Next, the process proceeded to Step 250, and a model having the best accuracy as the result of cross validation was selected.

Herein, a stepwise method is used. The stepwise method includes three kinds of a forward selection method, a stepwise forward selection method, and a backward selection method. The threshold value may be adjusted to a threshold value of P<0.05, and variable may be added. Therefore, the model having the best accuracy can be selected by forming a model many times at a larger loop 2 in FIG. 3.

Specifically, for evaluation of the MLR model, values of risk of pancreatic cancer (PC) with respect to saliva of breast cancer, oral cancer (CP), and IPMN were calculated. A group of the healthy subjects (C), and the subjects with CP and IPMN was formed. An AUC value that could identify pancreatic cancer from this group was calculated. The data were randomly divided into 10, a model was formed using 90% of the data, and the model was evaluated by the rest values of 10%. This operation was repeated 10 times. All the cases were selected once for evaluation, and cross validation (CV) of collecting the evaluation data and calculating the AUC value was performed.

10. Results of Model of Distinguishing Pancreatic Cancer from Searched Substance

FIG. 2 shows substances that exhibited high correlation values with the metabolites quantitatively determined at Step 120 in FIG. 1 at Step 142. In FIG. 2, a line is drawn between substances having R≧0.8. Eight clusters (groups of metabolites) are confirmed, but a cluster on the far left upper side in the drawing contains the most substances. In the cluster, alanine (Ala) forms the most networks with other substances. Therefore, alanine is determined as a metabolite for normalizing the concentration of the whole saliva. The metabolite used for normalization is not limited to the substance forming the most networks with other substances. For example, the total concentration of the metabolites, the sum of signals obtained during measurement of saliva by CE-MS (total ion electropherogram), or the area of a peak that is at a central order when all detected signals are sorted according to size may be used for normalization. The variable selection and the mathematical model are not limited to the stepwise method and the MLR model, respectively.

For example, for the variable selection, a correlation-based feature subset method (see M. A. Hall (1998). Correlation-based Feature Subset Selection for Machine Learning. Hamilton, New Zealand.), a relief method (see Marko Robnik-Sikonja, Igor Kononenko (1997). An adaptation of Relief for attribute estimation in regression. In: Fourteenth International Conference on Machine Learning, 296-304.), an SVM valiable selection method (see I. Guyon, J. Weston, S. Barnhill, V. Vapnik (2002). Gene selection for cancer classification using support vector machines. Machine Learning. 46(1-3): 389-422.), or the like may be applied.

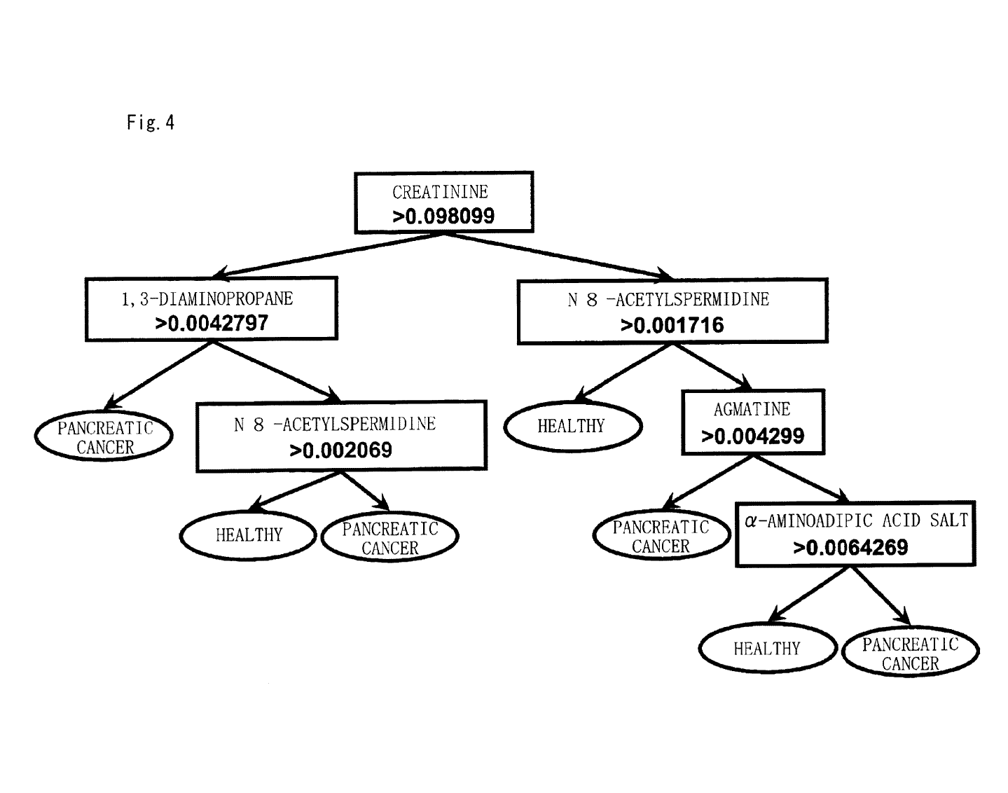

For the mathematical model, a mechanical learning method of dividing two groups may be applied. For example, Bayesian estimate (see Berger, James 0 (1985). Statistical Decision Theory and Bayesian Analysis. Springer Series in Statistics (Second ed.). Springer-Verlag. ISBN 0-387-96098-8.), neural network (ANN) (see D. E. Rumelhart, G. E. Hinton, and R. J. Williams, (1986): Learning representations by back-propagating errors, Nature, 323-9, 533-536.), support vector machine (SVM) (see J. Platt (1998) Fast Training of Support Vector Machines using Sequential Minimal Optimization. In B. Schoelkopf and C. Burges and A. Smola, editors, Advances in Kernel Methods-Support Vector Learning), Alternative decision tree (ADTree) (see Yoav Freund and Llew Mason (1999) The Alternating Decision Tree Algorithm. Proceedings of the 16th International Conference on Machine Learning, 124-133, and Freund, Y., Mason, L. (1999) The alternating decision tree learning algorithm. In: Proceeding of the Sixteenth International Conference on Machine Learning, Bled, Slovenia, 124-133), decision tree (see Ross Quinlan (1993). C4.5: Programs for Machine Learning. Morgan Kaufmann Publishers, San Mateo, Calif.), PART model (see Eibe Frank, Ian H. Witten (1998) Generating Accurate Rule Sets Without Global Optimization. In: Fifteenth International Conference on Machine Learning, 144-151), Random forest, PLS discriminant analysis (see Partial least squares-discriminant analysis; PLS-DA)(Lindgren, F; Geladi, P; Wold, S (1993). The kernel algorithm for PLS. J. Chemometrics 7: 45-59. doi: 10.1002/cem.1180070104.), Orthogonal PLS discriminant analysis (OPLS-DA) (see Trygg, J., & Wold, S. (2002). Orthogonal projections to latent structures (O-PLS). Journal of Chemometrics, 16(3), 119-128, and Breiman, Leo (2001). Random Forests. Machine Learning 45 (1): 5-32. doi: 10.1023/A: 1010933404324.), or the like may be applied. Bootstrap method and Bagging method (see Breiman, Leo (1996) Bagging predictors. Machine, Learning24 (2): 123-140) in which prediction is performed using average and majority of predictive values of a plurality of mathematical models that are obtained by forming a plurality of mechanical learning methods of dividing two groups may be used. Further, separation may be performed using a principal component in principal component analysis (Principal Component Analysis; PCA) (see Hotelling, H. (1933). Analysis of a complex of statistical variables into principal components. Journal of Educational Psychology, 24, 417-441) that is unsupervised learning. FIG. 4 is a model of decision tree that distinguishes the subjects with pancreatic cancer from the healthy subjects. The concentrations of metabolites that were normalized with a concentration marker (in this case, Ala) were used. The area under the ROC curve was 0.856 and the area under the ROC curve during 10-fold cross validation was 0.653.

Substances having a high ability of distinguishing the subjects with pancreatic cancer from the healthy subjects at Step 152 of the above section 9 are shown in Table 2.

TABLE 2

| | CONCENTRATION | | | | | | | | | | |
|---|---|---|---|---|---|---|---|---|---|---|---|
| | NORMALIZED WITH ALA (NO UNIT) | | | | | | | | | | |
| | HEALTHY SUBJECT | | PANCREATIC CANCER | | DETECTION RATIO (%) | | | | | | |
| NAME OF | | STANDARD | | STANDARD | HEALTHY | PANCREATIC | MANN-WHITNEY TEST | | ROC CURVE | | |
| SUBSTANCE | AVERAGE | DEVIATION | AVERAGE | DEVIATION | SUBJECT | CANCER | P VALUE | Q VALUE | AREA | 95% CI | P VALUE |
| N8-ACETYLSPERMIDINE | 0.0015 | 0.0015 | 0.0049 | 0.0071 | 70 | 80 | 1.55773E−06 | 0.000190043 | 0.7175 | 0.6414 to 0.7936 | 0.0001 |
| CREATININE | 0.1793 | 0.1349 | 0.1026 | 0.0945 | 100 | 99 | 1.74565E−05 | 0.001064849 | 0.695 | 0.6140 to 0.7759 | 0.0001 |
| SPERMINE | 0.0129 | 0.0222 | 0.0318 | 0.0477 | 63 | 83 | 3.99507E−05 | 0.001624661 | 0.6852 | 0.6036 to 0.7669 | 0.0001 |
| ASPARTIC ACID | 0.5559 | 0.2454 | 0.4290 | 0.2803 | 98 | 100 | 9.5103E−05 | 0.002320512 | 0.6772 | 0.5965 to 0.7578 | 0.0001 |
| N1-ACETYLSPERMIDINE | 0.0237 | 0.0147 | 0.0407 | 0.0351 | 97 | 99 | 0.000141561 | 0.002878407 | 0.6727 | 0.5929 to 0.7526 | 0.0001424 |
| N1-ACETYLSPERMINE | 0.0019 | 0.0045 | 0.0046 | 0.0060 | 30 | 60 | 7.37102E−05 | 0.002243162 | 0.6579 | 0.5868 to 0.7490 | 0.0002175 |
| CYTIDINE | 0.0232 | 0.0320 | 0.0107 | 0.0198 | 79 | 56 | 0.000175296 | 0.00305516 | 0.6663 | 0.5828 to 0.7499 | 0.0002494 |
| α-AMINOADTPIC ACID | 0.0381 | 0.0234 | 0.0689 | 0.0811 | 97 | 99 | 0.000784335 | 0.010632096 | 0.6525 | 0.5712 to 0.7337 | 0.0007859 |
| CYTOSINE | 0.0042 | 0.0094 | 0.0105 | 0.0180 | 35 | 61 | 0.000495928 | 0.007562907 | 0.6489 | 0.5662 to 0.7316 | 0.001037 |
| BETAINE | 0.2236 | 0.3431 | 0.1548 | 0.4208 | 98 | 98 | 0.001209678 | 0.013416426 | 0.5469 | 0.5614 to 0.7325 | 0.001211 |
| UREA | 39.1920 | 43.7834 | 23.5791 | 42.6852 | 98 | 98 | 0.001345148 | 0.013675675 | 0.6455 | 0.5595 to 0.7316 | 0.001347 |
| HOMOVANILLIC ACID | 0.0653 | 0.1133 | 0.0999 | 0.1260 | 46 | 75 | 0.00108606 | 0.013249934 | 0.845 | 0.5573 to 0.7327 | 0.001404 |
| N-ACETYLNEURAMINIC ACID | 1.6138 | 1.8792 | 0.9916 | 1.3150 | 92 | 95 | 0.00158931 | 0.014559781 | 0.6433 | 0.5560 to 0.7307 | 0.001592 |
| CYSTINE | 0.0040 | 0.0062 | 0.0068 | 0.0071 | 43 | 70 | 0.001670795 | 0.014559781 | 0.0381 | 0.5526 to 0.7236 | 0.002351 |
| UROCANIC ACID | 0.0965 | 0.0780 | 0.0696 | 0.0626 | 98 | 97 | 0.003836014 | 0.029249607 | 0.5313 | 0.5458 to 0.7167 | 0.003834 |
| FUMARIC ACID | 0.0104 | 0.0209 | 0.0206 | 0.0294 | 35 | 59 | 0.00304094 | 0.024732975 | 0.6262 | 0.5418 to 0.7105 | 0.005451 |
| 1,3-DIAMINOPROPANE | 0.0516 | 0.0554 | 0.0304 | 0.0397 | 78 | 73 | 0.00511035 | 0.034103773 | 0.8261 | 0.6368 to 0.7154 | 0.005477 |
| HYPOTAURINE | 0.0322 | 0.0508 | 0.0498 | 0.0739 | 51 | 78 | 0.004991032 | 0.034103773 | 0.6255 | 0.5352 to 0.7157 | 0.005712 |
| NICOTINIC ACID | 0.2649 | 1.1623 | 0.1163 | 0.5318 | 75 | 65 | 0.005311243 | 0.034103773 | 0.6246 | 0.5365 to 0.7127 | 0.006067 |
| AGMATINE | 0.0050 | 0.0050 | 0.0033 | 0.0039 | 81 | 59 | 0.005639892 | 0.034403344 | 0.6244 | 0.5377 to 0.7112 | 0.006123 |
| VALINE | 0.3794 | 0.2213 | 0.4894 | 0.3424 | 100 | 99 | 0.006972284 | 0.040119018 | 0.6225 | 0.5380 to 0.7070 | 0.006964 |
| 2-HYDROXY-4-METHYLPENTANOIC ACID | 0.0645 | 0.0686 | 0.0949 | 0.0948 | 75 | 94 | 0.007234577 | 0.040119018 | 0.6218 | 0.5333 to 0.7103 | 0.007288 |
| ALANYL-ALANINE | 0.0301 | 0.0175 | 0.0247 | 0.0139 | 89 | 86 | 0.010929763 | 0.055559628 | 0.6155 | 0.5261 to 0.7049 | 0.01092 |
| CITRIC ACID | 0.3689 | 0.5593 | 0.2466 | 0.5154 | 95 | 85 | 0.014695008 | 0.067709323 | 0.6107 | 0.5265 to 0.6949 | 0.01474 |
| GLUCOSAMINE | 0.0124 | 0.0117 | 0.0078 | 0.0104 | 85 | 55 | 0.01143552 | 0.055805337 | 0.6108 | 0.5209 to 07004 | 0.0148 |
| CARNOSINE | 0.0029 | 0.0046 | 0.0016 | 0.0040 | 54 | 38 | 0.008103017 | 0.042981222 | 0.609 | 0.5195 to 0.8985 | 0.01629 |
| GLYCINE-GLYCINE | 0.0154 | 0.0158 | 0.0229 | 0.0202 | 56 | 80 | 0.015589845 | 0.067709323 | 0.6086 | 0.5203 to 0.6968 | 0.01677 |
| 2-AMINOBUTYRIC ACID | 0.0758 | 0.1971 | 0.0723 | 0.0719 | 95 | 100 | 0.017659224 | 0.074290531 | 0.6077 | 0.5190 to 0.6965 | 0.01762 |
| ARGININE | 0.5163 | 0.3841 | 0.4082 | 0.4195 | 100 | 99 | 0.019323999 | 0.078584264 | 0.6062 | 0.5197 to 0.6927 | 0.01928 |
| N-ACETYLGLUTAMIC ACID | 0.0035 | 0.0057 | 0.0053 | 0.0060 | 40 | 63 | 0.015062166 | 0.067709323 | 0.6052 | 0.5172 to 0.6933 | 0.02041 |
| GLYCEROPHOSPHORIC ACID | 0.3663 | 0.2880 | 0.3039 | 0.3335 | 100 | 99 | 0.020954571 | 0.082466378 | 0.6048 | 0.5208 to 0.6889 | 0.02091 |
| PHOSPHOENOLPYRUVIC ACID | 0.0130 | 0.0194 | 0.0222 | 0.0398 | 44 | 67 | 0.026459721 | 0.100877686 | 0.5972 | 0.5093 to 0.6852 | 0.03216 |
| ISOLEUCINE | 0.1286 | 0.0693 | 0.1629 | 0.1046 | 100 | 100 | 0.037465083 | 0.138507278 | 0.5945 | 0.5089 to 0.6800 | 0.003738 |
| ADENOSINE | 0.0126 | 0.0131 | 0.0093 | 0.0141 | 81 | 78 | 0.039739367 | 0.142594198 | 0.593 | 0.5040 to 0.6820 | 0.04054 |
| GUANINE | 0.0520 | 0.0403 | 0.0449 | 0.0543 | 94 | 91 | 0.04241365 | 0.147344023 | 0.5921 | 0.5085 to 0.6778 | 0.04236 |
| DIHYDROXYACETONEPHOSPHORIC ACID | 0.2462 | 0.1806 | 0.2041 | 0.1814 | 94 | 97 | 0.047321948 | 0.154153689 | 0.5901 | 0.5043 to 0.6758 | 0.04722 |
| CADAVERINE | 0.5943 | 0.7614 | 0.9336 | 1.2717 | 100 | 99 | 0.048015084 | 0.154153689 | 0.5898 | 0.5039 to 0.5757 | 0.0479 |

In Table 2, a detection ratio shows a ratio of cases in which a peak can be detected relative to all the cases in each group of the healthy subjects and the subjects with pancreatic cancer. A 95% confidential interval (CI) represents a value of 95% confidential interval.

A Mann-Whitney test that is a non-parametric two-group test for the healthy subject group and the pancreatic cancer group was performed between the healthy subjects and the subjects with pancreatic cancer, the p value of each of the metabolites was calculated, and the p value was corrected with the false discovery rate (FDR). A test of q value was performed.

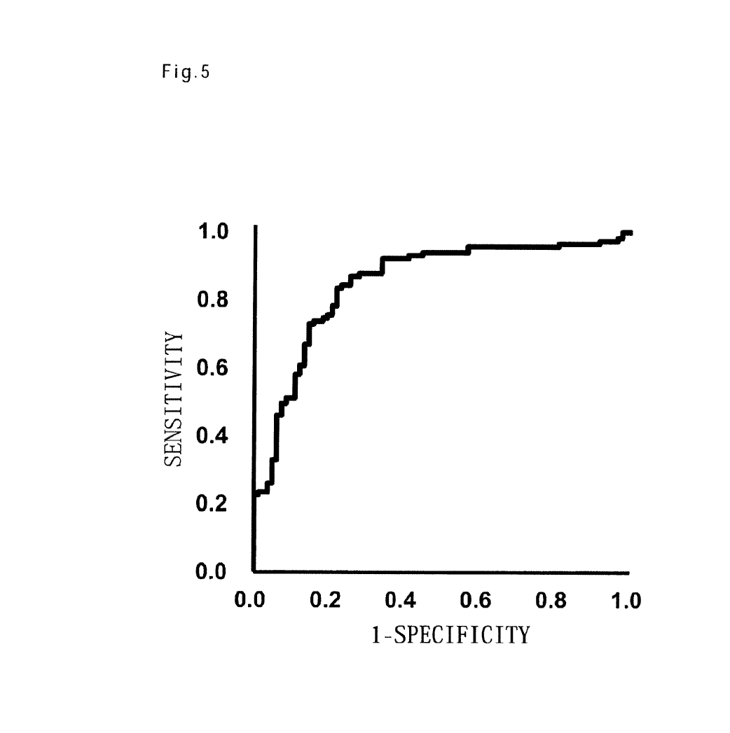

For evaluation of sensitivity and specificity of the substances that distinguish two groups of the subjects with pancreatic cancer (PC) and the healthy subjects (C), receiver operating characteristic (ROC) analysis was performed. The results are shown in FIG. 5. The concentrations of metabolites that were normalized with the concentration marker were used. The substances contained in the MLR model and a parameter and an odds ratio thereof are shown in Table 3.

TABLE 3

| ITEM | PARAMETER | P VALUE | 95% CI | | ODDS RATIO | 95% CI | |
|---|---|---|---|---|---|---|---|
| (INTERCEPT) | −0.0375549 | 0.9179 | −0.7551751 | 0.67987596 | — | — | — |
| CREATININE | 8.81954122 | <.0001 | 5.11051632 | 13.1970776 | 6765.16 | 165.7559 | 538788.1 |
| N1-ACETYLSPERMIDINE | −36.266806 | 0.0017 | −80.649929 | −14.849848 | 1.78E−16 | 4.57E−27 | 3.56E−07 |
| α-AMINOADIPIC ACID | −25.266236 | 0.0039 | −43.368968 | −9.1982815 | 1.04E−11 | 1.48E−19 | 0.000101 |
| N-ACETYLNEURAMINIC | 0.30754159 | 0.0219 | 0.05238651 | 0.58469864 | 1.360077 | 1.053783 | 1.79445 |
| ACID | | | | | | | |
| 1,3-DIAMINOPROPANE | 11.309134 | 0.004 | 4.02635127 | 19.6651022 | 81563.25 | 56.058 | 3.47E+08 |

The area under the ROC curve in FIG. 5 was 0.8763 (95% CI: 0.8209 to 0.9317, p<0.0001). The sensitivity of optimal cut-off value was 0.8348, and (1-specificity) was 0.2169.

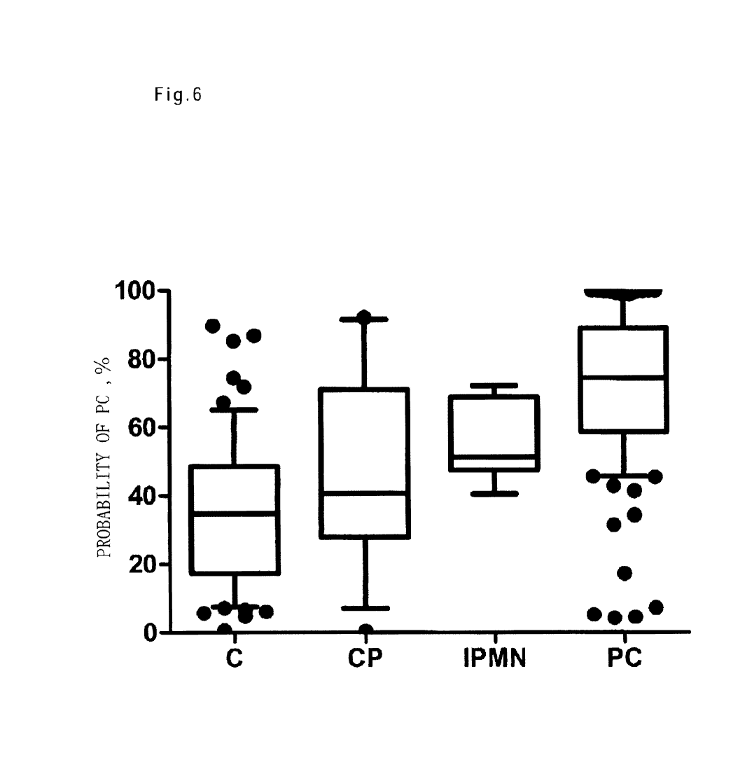

Using the MLR model that can distinguish the healthy subjects and the subjects with pancreatic cancer, values calculated as risk of pancreatic cancer (PC) of the healthy subjects (C) and the subjects with pancreatic cancer (PC) as well as the patients with breast cancer, oral cancer (CP), and IPMN are shown in FIG. 6.

FIG. 6 is a diagram in which the risk of pancreatic cancer (PC) of C, PC, breast cancer, oral cancer (CP), and IPMN is plotted by the model of classifying the healthy subjects (C) and the subjects with pancreatic cancer (PC). A boxplot represents values of 10%, 25%, 50%, 75%, and 90% from the top, and values under 10% and values beyond 90% are expressed as plots.

Table 4 shows an AUC value in which the specificity and general-purpose properties of the MLR model were evaluated.

TABLE 4

| | | DISTINGUISH |
|---|---|---|
| | DISTINGUISH | PC FROM C + |
| | PC FROM C | CP + IPMN |
| WHEN ALL THE | 0.88 | 0.85 |
| DATA ARE USED | | |
| IN CASE OF CV | 0.86 | 0.83 |

Herein, CV represents a case of cross validation.

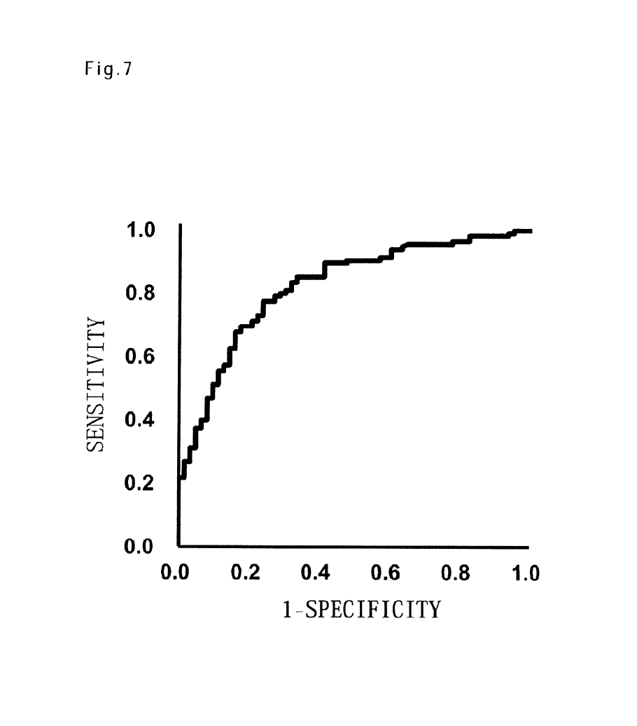

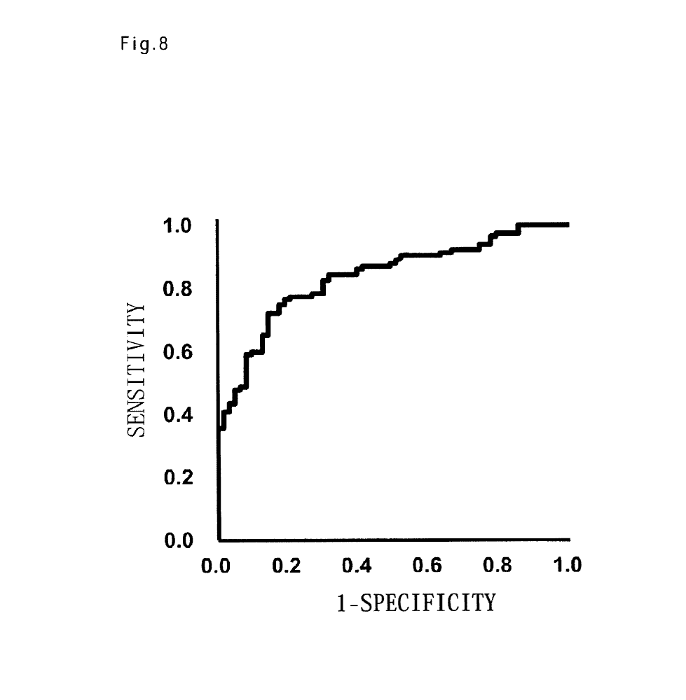

In FIG. 7, an ROC curve at which the MLR model was formed using the absolute concentration without concentration correction is shown. Table 5 shows selected markers and coefficients. The area under the ROC curve was 0.8264 (95% CI: 0.7619 to 0.8874, p<0.0001). The accuracy was slightly decreased as compared with a case with concentration correction, but highly accurate prediction was possible. For formation of this model, a P value at which the variable was added was 0.05, and a P value at which the variable was eliminated was 0.05 using a stepwise forward selection method of stepwise variable selection. FIG. 8 shows an ROC curve at which the P value at which the variable was added was 0.05 using a forward selection method as a method of variable selection. Table 6 shows selected markers and coefficients. The area under the ROC curve was 0.8373 (95% CI: 0.7792 to 0.8954, p<0.0001). Regardless of use of the markers and coefficients that were different from the model of FIG. 7, prediction accuracy of the same level could be achieved.

TABLE 5

| ITEM | PARAMETER | P VALUE | 95% CI | | ODDS RATIO | 95% CI | |
|---|---|---|---|---|---|---|---|
| (INTERCEPT) | 0.40019 | 0.162 | −0.15107 | 0.977355 | — | — | — |
| α-AMINOADIPIC | −0.64356 | <.0001 | −0.95539 | −0.39782 | 0.525421 | 0.384661 | 0.671782 |
| ACID | | | | | | | |
| PHOSPHORYLCHOLINE | −0.04641 | 0.0427 | −0.09647 | −0.00235 | 0.954646 | 0.90804 | 0.997649 |
| N-ACETYLNEURAMINIC | 0.014466 | <.0001 | 0.007874 | 0.02224 | 1.014571 | 1.007905 | 1.022489 |
| ACID | | | | | | | |

TABLE 6

| ITEM | PARAMETER | P VALUE | 95% CI | | ODDS RATIO | 95% CI | |
|---|---|---|---|---|---|---|---|
| (INTERCEPT) | 0.277503 | 0.3624 | −0.31567 | 0.884388 | — | — | — |
| 3PG | 0.166107 | 0.0028 | 0.063545 | 0.282987 | 1.1807 | 1.065608 | 1.327088 |
| N1-ACETYLSPERMINE | −2.26566 | 0.0127 | −4.28489 | −0.65321 | 0.103762 | 0.013775 | 0.520371 |
| α-AMINOADIPIC | −0.9899 | <.0001 | −1.47201 | −0.58351 | 0.371613 | 0.229465 | 0.557935 |
| ACID | | | | | | | |
| N-ACETYLNEURAMINIC | 0.012425 | 0.0007 | 0.005686 | 0.020249 | 1.012503 | 1.005702 | 1.020456 |
| ACID | | | | | | | |
| 4-(β- | 4.469352 | 0.0041 | 1.397495 | 7.786111 | 87.30013 | 4.045055 | 2406.938 |
| ACETYLAMINOETHYL)IMIDAZOLE | | | | | | | |

### 11. Consideration of Searched Substance

In the present invention, the concentrations of ionic metabolites contained in saliva were simultaneously measured, and markers having a high ability of distinguishing the subjects with pancreatic cancer from the healthy subjects were selected. Further, a model having higher accuracy (sensitivity and specificity) as compared with a single substance could be developed by combining the markers.

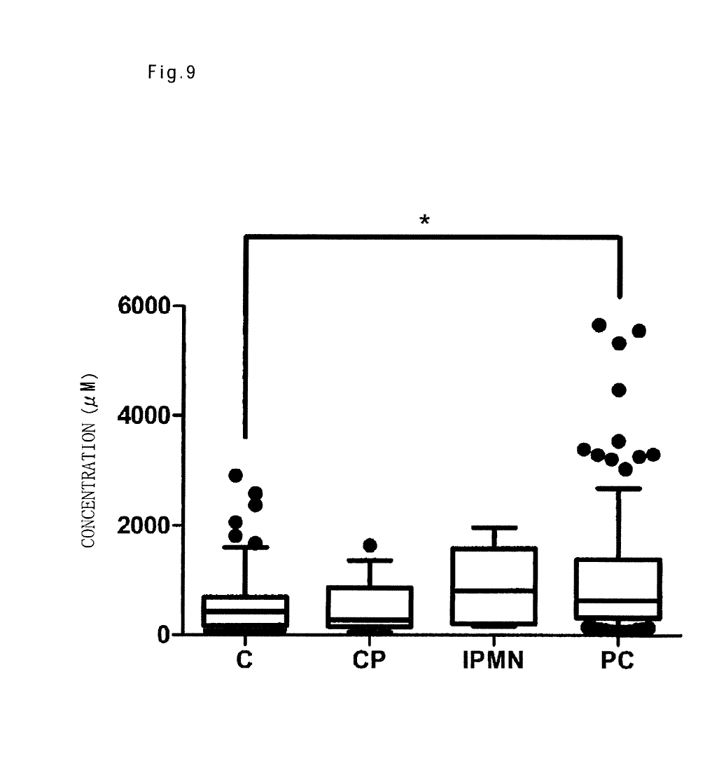

A problem involved in using saliva is that there is a greater variation of concentrations present in saliva as compared with blood. In this method, saliva was collected under unified conditions depending on the collection time and dietary restriction before the collection. Some of the samples had trends in which the concentrations of all the substances were clearly high or low. FIG. 9 shows a difference in the total concentration of amino acids in saliva of each disease. A boxplot has the same meanings as that of FIG. 6. A Kruskal-Wallis test that is a non-parametric multiplex test was performed, and the P value was 0.0138. After that, a Dunn's post test was performed. Only between C and PC, the P value was less than 0.05.

The concentration in the subjects with pancreatic cancer (PC) is significantly higher than that in the healthy subjects (C), and as an indication exhibiting a risk of pancreatic cancer, a high total concentration in saliva itself may be used. However, some of the samples of C have a concentration higher than PC, and in contrast, some of the samples of PC have a concentration lower than C. Therefore, when the samples are simply considered for a risk, the accuracy is low (from results of ROC analysis between C and PC in data of FIG. 8, AUC=0.6282, and p=0.004756).

When only a sample having a concentration falling within a certain range except for the samples (the samples of C having higher concentration, and the samples of PC having lower concentration) is a target of a test, based on the whole concentration, examination should be omitted. Therefore, the fluctuation of the entire concentration was offset by performing normalization using a substance that has a high correlation with the entire metabolite concentration of the saliva and can be detected in all the samples by the method shown in FIG. 2. The normalization may be omitted.

Among the substances as marker candidates in Table 2, polyamines such as spermine, and acetylated polyamines such as N8-acetylspermidine, N1-acetylspermidine, and N1-acetylspermine are each a substance that reflects on a state of the pancreatic tissues according to various changes in cancer. However, for example, in a case of spermine in urine, the concentration correction with creatinine is only considered. Therefore, spermine cannot achieve the accuracy that a tumor marker measured in a blood test can achieve. Because polyamines in blood are taken up by erythrocytes (see Fu N N, Zhang H S, MaM, Wang H. (2007) Quantification of polyamines in human erythrocytes using a new near-infrared cyanine 1-(epsilon-succinimidyl-hexanoate)-1′-methyl-3,3,3′,3′-tetramethyl-indocarbocyanine-5,5′-disulfonate potassium with CE-LIF detection. Electrophoresis. 28(5): 822-9), the amount of polyamines in a free state is extremely small, and the concentration thereof in urine is extremely low. Even when polyamines in blood and urine are measured in breast cancer, the highest concentration of spermidine is about 140 nM (nanomol), and the concentration of N-acetylspermidine is about 64 nM (nanomol). Thus, concentrations that are much lower than the concentrations in saliva are reported (see Byun J A, Lee S H, Jung B H, Choi M H, Moon M H, Chung B C. (2008) Analysis of polyamines as carbamoyl derivatives in urine and serum by liquid chromatography-tandem mass spectrometry. Biomed Chromatogr. 22(1): 73-80). The quantitative determination of polyamines in erythrocytes requires a complicated step. Therefore, a diagnosis method found in the present invention has characteristics in which a highly accurate prediction can be achieved due to the contribution of the following three points, including (i) use of saliva capable of detecting the marker substances at high concentration, (ii) a decrease in dispersion generated at each measurement due to a simple treatment process for measurement, and (iii) use of the mathematical model in combination with the markers. A difference in mRNA in saliva between the patients with pancreatic cancer and the healthy subjects is already known (Non-Patent Literature 4). However, mRNA is completely different because a molecular group to which the present invention is directed is a metabolite. The variation of metabolites by themselves in saliva depending on pancreatic cancer is already known (Non-Patent Literature 5). However, substances that are not disclosed in known documents are used as a marker in the present invention, and a mathematical model for eliminating the effect of a specific concentration variation in saliva and identifying pancreatic cancer with high sensitivity and specificity can be developed.

With respect to four groups of healthy subjects, chronic pancreatitis, IPMN, and pancreatic cancer, a distribution of risk of pancreatic cancer that is predicted by the MLR model shows that the model exhibits high specificity for pancreatic cancer (FIG. 6). The results of cross validation (Table 4) and the results of a test for distinguishing the pancreatic cancer group from the groups other than the pancreatic cancer group also show that this model has high sensitivity and specificity that cannot be achieved by the conventional method.

In Examples, capillary electrophoresis-mass spectroscopy (CE-MS) is used to measure the concentrations of metabolites in saliva. However, high speed liquid chromatography (LC), gas chromatography (GC), chip LC, or chip CE, or GC-MS, LC-MS, and CE-MS methods in which they are combined with a mass spectrometer (MS), a measurement method for each MS alone, an NMR method, a measurement method for a metabolite substance that is derivatized into a fluorescent substance or a UV absorptive material, or an enzyme method in which an antibody is produced and measured by an ELISA method, may be used. Regardless of the measurement method, measurement may be performed by any analysis.

Next, a biomarker for breast cancer will be described.

Cases included healthy subjects (20 cases), and patients with breast cancer (90 cases) including patients with breast cancer before initiation of treatment (37 cases), patients with breast cancer that were treated with chemotherapy, hormonotherapy, or the like. In the breast cancer cases, one patient was male and the rest were female. In the patients with breast cancer before initiation of treatment, eight cases were DCIS, and 29 cases were invasive ductal carcinoma.

A method for collecting saliva, a method for measuring metabolites, and the like were the same as those used in the biomarker for pancreatic cancer.

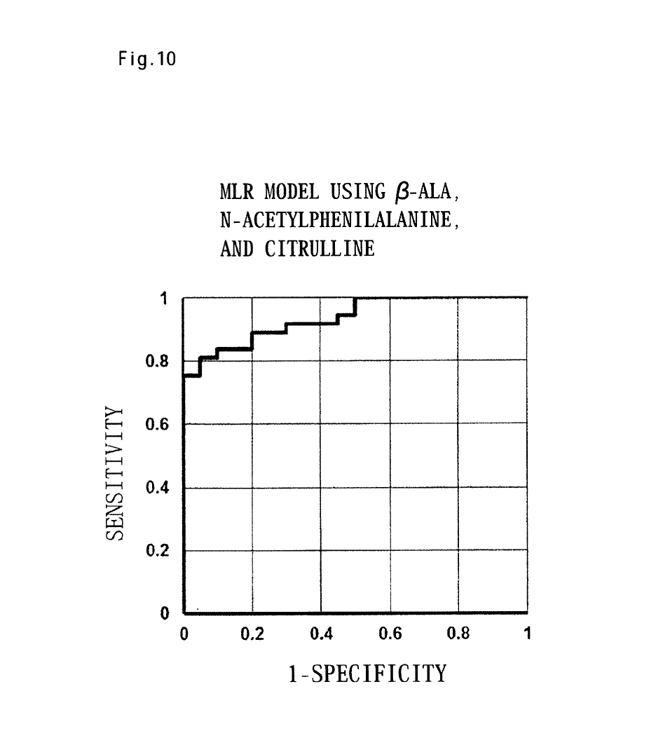

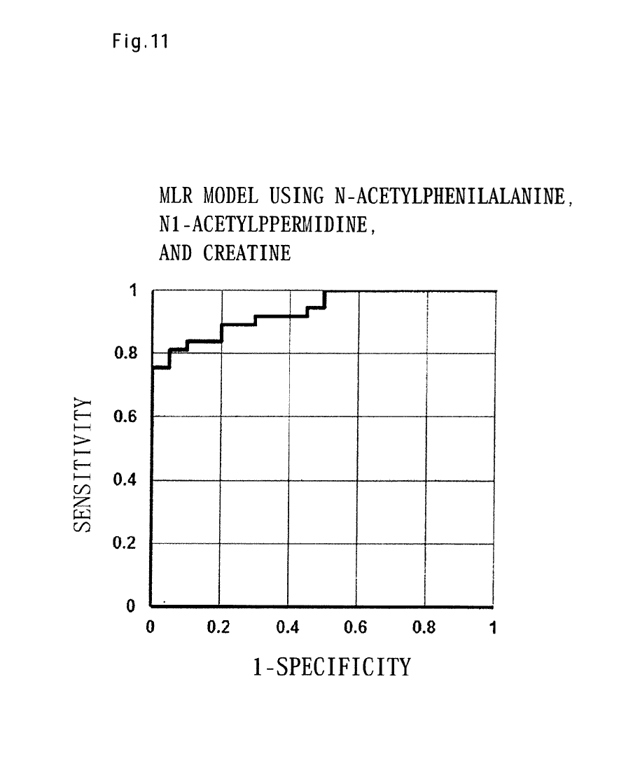

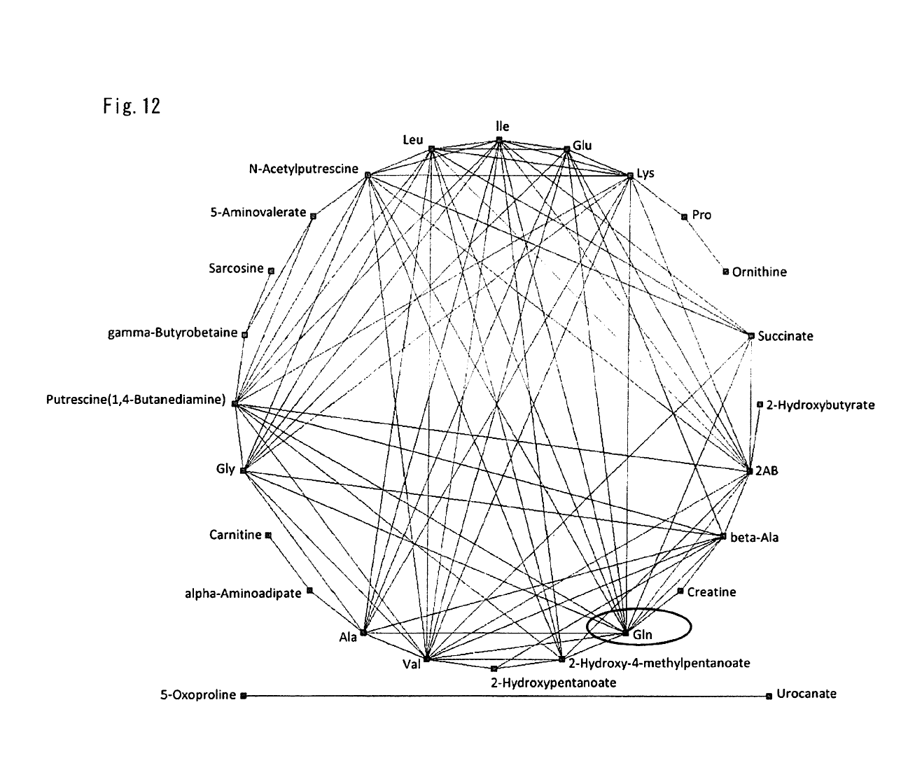

In a variable selection method performed during formation of multiple logistic regression model (MLR mode), only a substance of Q 0.05 was used. In FIG. 10, β-alanine (Beta-Ala), N-acetylphenylalanine (N-Acetylphenylalanine), and citrulline (Citrulline) were used for addition of variable at P≦0.05 or elimination of variable at P≧0.05 by a stepwise forward selection method. In FIG. 11, N-acetylphenylalanine, N1-acetylspermidine, and creatine (Creatine), which include N1-acetylspermidine (N1-Acetylspermidine) that is a known marker, were used in a method for adding (increasing) a variable at P≦0.05 by a stepwise forward selection method. In the model of FIG. 10, as the ROC value of each substance, the ROC value of β-alanine is 0.8373, the ROC value of N-acetylphenylalanine is 0.7122, and the ROC value of citrulline is 0.698. By conversion of the substances into the MLR model, the ROC values are increased to 0.9622. Also in FIG. 12 including a known marker, the ROC value of N-acetylphenylalanine is 0.7122, the ROC value of N-acetylspermidine is 0.7811, and the ROC value of creatine is 0.7824. It was confirmed that the ROC values were increased to 0.9365 by combination of the substances using the MLR model.

Substances in which the absolute concentration exhibits a statistic significant difference (p<0.05 in the Mann-Whitney test) between the patients with breast cancer and the healthy subjects are shown in Tables 7-1, 7-2, 7-3, and 7-4. Comparison was performed in 20 cases of the healthy subjects and all the cases (90 cases) including the patients with breast cancer before treatment without chemotherapy or hormonotherapy (37 cases). A Q value was calculated by the false discovery rate (FDR).

TABLE 7-1

CONCENTRATION (μM)

| | |
|---|---|
| BREAST CANCER | DETECTION RATIO (%) |

| | | | | ONLY BEFORE | | INCLUDING DURING | | | BREAST CANCER | |
|---|---|---|---|---|---|---|---|---|---|---|
| | | HEALTHY SUBJECT | | TREATMENT | | TREATMENT | | | | INCLUDING |
| | | | STANDARD | | STANDARD | | STANDARD | HEALTHY | BEFORE | DURING |
| NAME OF SUBSTANCE | | AVERAGE | DEVIATION | AVERAGE | DEVIATION | AVERAGE | DEVIATION | SUBJECT | TREATMENT | TREATMENT |
| CHOLINE | Choline | 5.378 | 3.347 | 20.534 | 26.7069 | 18.249 | 21.29059 | 20 | 37 | 90 |
| 2-HYDROXYBUTYRIC ACID | 2-Hydroxybutyrate | 1.027 | 0.713 | 2.6841 | 4.1045 | 2.5884 | 3.078189 | 18 | 37 | 90 |
| β-ALANINE | beta-Ala | 1.233 | 0.919 | 5.4375 | 9.96858 | 4.5107 | 7.349677 | 17 | 36 | 87 |
| 3-METHYLHISDINE | 3-Methylhistidine | 0.085 | 0.180 | 1.2255 | 2.20901 | 1.0247 | 1.925432 | 6 | 28 | 70 |
| α-AMINOBUTYRIC ACID | 2AB | 0.791 | 0.703 | 6.2284 | 20.2287 | 5.3851 | 16.0949 | 16 | 35 | 86 |
| CADAVERINE | Cadaverine | 9.083 | 13.402 | 35.46 | 41.1071 | 39.765 | 55.12648 | 19 | 37 | 89 |
| N-ACETYL-β-ALANINE | N-Acetyl-beta-alanine | 0.282 | 0.374 | 1.4267 | 2.0143 | 1.2751 | 1.666246 | 9 | 32 | 76 |
| ISETHIONIC ACID | Isethionate | 0.148 | 0.109 | 0.3581 | 0.27681 | 0.4044 | 0.804686 | 14 | 32 | 77 |
| N-ACETYLPHENYLALANINE | N-Acetylphenylalanine | 0.086 | 0.116 | 0.0172 | 0.07346 | 0.0229 | 0.074225 | 10 | 3 | 10 |
| TRIMETHYLLYSINE | N6,N6,N6-Trimethyllysine | 0.035 | 0.089 | 0.2699 | 0.36582 | 0.2515 | 0.342463 | 3 | 25 | 57 |
| α-AMINOADIPIC ACID | alpha-Aminoadipate | 0.883 | 0.814 | 3.4689 | 5.42307 | 3.0669 | 6.266668 | 17 | 34 | 83 |
| SPERMINE | Spermine | 0.065 | 0.119 | 1.3403 | 3.39481 | 1.0308 | 2.855626 | 6 | 28 | 60 |
| N1-ACETYLSPERMIDINE | N1-Acetylspermidine | 0.327 | 0.345 | 1.289 | 1.74192 | 1.1973 | 1.595473 | 15 | 35 | 87 |
| CREATINE | Creatine | 10.968 | 6.522 | 34.274 | 63.8828 | 28.365 | 50.60692 | 20 | 37 | 90 |
| γ-BUTYROBETAINE | gamma-Butyrobetaine | 1.914 | 1.542 | 7.3809 | 11.9304 | 6.1926 | 8.983804 | 20 | 36 | 89 |
| SARCOSINE | Sarcosine | 5.133 | 4.294 | 13.808 | 18.2185 | 10.827 | 13.26566 | 20 | 37 | 90 |
| PYRUVIC ACID | Pyruvate | 40.919 | 27.286 | 98.681 | 93.8728 | 105.45 | 133.2851 | 20 | 37 | 90 |
| UROCANIC ACID | Urocanate | 0.995 | 1.082 | 5.054 | 13.3985 | 3.9576 | 8.875522 | 13 | 32 | 82 |
| PIPERIDINE | Piperidine | 0.073 | 0.152 | 0.3755 | 0.53702 | 0.7133 | 2.011631 | 7 | 25 | 67 |
| SERINE | Ser | 10.300 | 9.622 | 34.007 | 67.4699 | 28.451 | 47.13016 | 20 | 37 | 90 |
| HOMOVANILLIC ACID | Homovanillate | 1.721 | 0.906 | 3.8279 | 3.74161 | 4.2265 | 5.204739 | 20 | 37 | 88 |
| 5-OXOPROLINE | 5-Oxoproline | 6.788 | 12.685 | 18.15 | 44.9634 | 14.684 | 30.06842 | 20 | 37 | 90 |
| GABA | GABA | 1.286 | 1.299 | 2.5587 | 2.18752 | 4.4657 | 16.60331 | 20 | 36 | 89 |
| 5-AMINOVALERIC ACID | 5-Aminovalerate | 195.795 | 209.963 | 827.22 | 1489.58 | 679.53 | 1052.043 | 20 | 37 | 89 |
| TRIMETHYLAMINE-N-OXIDE | Trimethylamine N-oxide | 0.091 | 0.195 | 0.4613 | 0.61489 | 0.4739 | 1.005668 | 4 | 24 | 61 |
| 2-HYDROXYVALERIC ACID | 2-Hydroxypentanoate | 5.843 | 4.793 | 16.188 | 25.3406 | 15.137 | 19.67004 | 20 | 37 | 90 |
| CARNITINE | Carnitine | 0.757 | 0.633 | 1.8914 | 2.68246 | 1.9392 | 4.54229 | 20 | 37 | 90 |
| ISOPROPANOLAMINE | Isopropanolamine | 0.530 | 0.806 | 2.2374 | 4.13934 | 2.047 | 3.62564 | 12 | 31 | 77 |
| THREONINE | Thr | 4.163 | 3.602 | 12.408 | 19.5441 | 12.486 | 19.3648 | 20 | 37 | 90 |
| HYPOTAURINE | Hypotaurine | 0.318 | 1.106 | 1.9495 | 2.79625 | 1.6802 | 2.564848 | 2 | 19 | 42 |
| LACTIC ACID | Lactate | 128.842 | 80.253 | 348.15 | 495.025 | 394.62 | 807.0465 | 20 | 37 | 90 |
| 2-HYDROXY-4- | 2-Hydroxy-4- | 2.068 | 2.293 | 6.7127 | 12.7565 | 5.9735 | 9.612041 | 20 | 36 | 89 |
| METHYLPENTANOIC ACID | methylpentanoate | | | | | | | | | |
| HYDROXYPROLINE | Hydroxyproline | 0.437 | 0.731 | 1.6485 | 3.26692 | 1.4398 | 2.683583 | 9 | 31 | 75 |

| | | MANN-WHITNEY TEST | | | | | |
|---|---|---|---|---|---|---|---|
| | | | | INCLUDING | | | |
| | | ONLY BEFORE | | DURING | | | |
| | | TREATMENT | | TREATMENT | | | |
| | | vs HEALTHY | | vs HEALTHY | | | |
| | | SUBJECT | | SUBJECT | | PUBLICLY | |
| NAME OF SUBSTANCE | | P VALUE | Q VALUE | P VALUE | Q VALUE | KNOWN | SIGNIFICANT |
| CHOLINE | Choline | 1.18E−05 | 0.001 | 3.07E−06 | 0.0002 | | 1 |
| 2-HYDROXYBUTYRIC ACID | 2-Hydroxybutyrate | 4.11E−05 | 0.002 | 4.39E−06 | 0.0002 | | 1 |
| β-ALANINE | beta-Ala | 3.34E−05 | 0.002 | 1.44E−05 | 0.0004 | | 1 |
| 3-METHYLHISDINE | 3-Methylhistidine | 6.88E−05 | 0.002 | 5.69E−06 | 0.0002 | | 1 |
| α-AMINOBUTYRIC ACID | 2AB | 8.39E−05 | 0.002 | 2.38E−05 | 0.0005 | | 1 |
| CADAVERINE | Cadaverine | 8.81E−05 | 0.002 | 2.13E−05 | 0.0005 | 78 | 1 |
| N-ACETYL-β-ALANINE | N-Acetyl-beta-alanine | 9.62E−05 | 0.002 | 4.60E−05 | 0.0008 | | 1 |
| ISETHIONIC ACID | Isethionate | 0.0001192 | 0.002 | 5.75E−05 | 0.0008 | | 1 |
| N-ACETYLPHENYLALANINE | N-Acetylphenylalanine | 0.0003692 | 0.004 | 7.54E−05 | 0.0009 | | 1 |
| TRIMETHYLLYSINE | N6,N6,N6-Trimethyllysine | 0.0003921 | 0.004 | 0.000271 | 0.0026 | | 1 |
| α-AMINOADIPIC ACID | alpha-Aminoadipate | 0.0004268 | 0.004 | 0.000311 | 0.0027 | | 1 |
| SPERMINE | Spermine | 0.0003262 | 0.004 | 0.001251 | 0.005 | 8 | 1 |
| N1-ACETYLSPERMIDINE | N1-Acetylspermidine | 0.0005128 | 0.005 | 0.00023 | 0.0024 | 8 | 1 |
| CREATINE | Creatine | 0.0004883 | 0.005 | 0.000933 | 0.0043 | | 1 |
| γ-BUTYROBETAINE | gamma-Butyrobetaine | 0.0006073 | 0.005 | 0.000385 | 0.0028 | | 1 |
| SARCOSINE | Sarcosine | 0.0006649 | 0.005 | 0.002504 | 0.0087 | | 1 |
| PYRUVIC ACID | Pyruvate | 0.0008749 | 0.006 | 0.000421 | 0.0029 | | 1 |
| UROCANIC ACID | Urocanate | 0.001326 | 0.009 | 0.000101 | 0.0011 | | 1 |
| PIPERIDINE | Piperidine | 0.0015033 | 0.01 | 5.83E−05 | 0.0008 | | 1 |
| SERINE | Ser | 0.0016657 | 0.01 | 0.000509 | 0.0033 | | 1 |
| HOMOVANILLIC ACID | Homovanillate | 0.0016108 | 0.01 | 0.000735 | 0.0041 | | 1 |
| 5-OXOPROLINE | 5-Oxoproline | 0.0018661 | 0.01 | 0.000369 | 0.0028 | | 1 |
| GABA | GABA | 0.0019086 | 0.01 | 0.000844 | 0.0042 | | 1 |
| 5-AMINOVALERIC ACID | 5-Aminovalerate | 0.002093 | 0.01 | 0.000323 | 0.0027 | | 1 |
| TRIMETHYLAMINE-N-OXIDE | Trimethylamine N-oxide | 0.0020797 | 0.01 | 0.000762 | 0.0041 | | 1 |
| 2-HYDROXYVALERIC ACID | 2-Hydroxypentanoate | 0.0023391 | 0.011 | 0.00086 | 0.0042 | | 1 |
| CARNITINE | Carnitine | 0.0025095 | 0.012 | 0.004876 | 0.0133 | | 1 |
| ISOPROPANOLAMINE | Isopropanolamine | 0.0030194 | 0.012 | 0.000882 | 0.0042 | | 1 |
| THREONINE | Thr | 0.0029044 | 0.012 | 0.001147 | 0.0048 | 7 | 1 |
| HYPOTAURINE | Hypotaurine | 0.0028193 | 0.012 | 0.003926 | 0.0116 | | 1 |
| LACTIC ACID | Lactate | 0.0029968 | 0.012 | 0.004292 | 0.0122 | | 1 |
| 2-HYDROXY-4- | 2-Hydroxy-4- | 0.0033304 | 0.013 | 0.001146 | 0.0048 | | 1 |
| METHYLPENTANOIC ACID | methylpentanoate | | | | | | |
| HYDROXYPROLINE | Hydroxyproline | 0.0035492 | 0.013 | 0.000991 | 0.0044 | | 1 |

TABLE 7-2

CONCENTRATION (μM)

| | |
|---|---|
| BREAST CANCER | DETECTION RATIO (%) |

| | | | | ONLY BEFORE | | INCLUDING DURING | | | BREAST CANCER | |
|---|---|---|---|---|---|---|---|---|---|---|
| | | HEALTHY SUBJECT | | TREATMENT | | TREATMENT | | | | INCLUDING |
| | | | STANDARD | | STANDARD | | STANDARD | HEALTHY | BEFORE | DURING |
| NAME OF SUBSTANCE | | AVERAGE | DEVIATION | AVERAGE | DEVIATION | AVERAGE | DEVIATION | SUBJECT | TREATMENT | TREATMENT |
| ALANINE | Ala | 21.390 | 19.430 | 67.526 | 117.162 | 69.232 | 120.485 | 20 | 37 | 90 |
| VALINE | Val | 11.610 | 15.718 | 49.326 | 129.44 | 41.908 | 95.743 | 20 | 37 | 90 |
| BUTYRIC ACID | Butanoate | 65.627 | 64.301 | 204.46 | 276.832 | 216.69 | 280.267 | 20 | 37 | 89 |
| SPERMIDINE | Spermidine | 1.339 | 1.342 | 3.874 | 5.26322 | 3.3277 | 4.61563 | 20 | 36 | 88 |
| N8-ACETYLSPERMIDINE | N8-Acetylspermidine | 0.013 | 0.031 | 0.0715 | 0.0938 | 0.0737 | 0.11937 | 3 | 20 | 49 |
| ADENINE | Adenine | 0.673 | 0.482 | 1.4111 | 1.21709 | 1.2015 | 1.05126 | 16 | 34 | 81 |
| PUTRESCINE | Putrescine(1,4- | 39.277 | 40.268 | 182.54 | 417.293 | 137.74 | 287.799 | 20 | 37 | 89 |
| | Butanediamine) | | | | | | | | | |
| N6-ACETYLLYSINE | N-epsilon-Acetyllysine | 0.077 | 0.173 | 0.346 | 0.42637 | 0.3251 | 0.41918 | 4 | 22 | 51 |
| 6-HYDROXYHEXANOIC ACID | 6-Hydroxyhexanoate | 0.949 | 1.156 | 0.2497 | 0.66804 | 0.4526 | 0.93174 | 9 | 5 | 19 |
| PROPIONIC ACID | Propionate | 212.587 | 228.193 | 546.67 | 646.293 | 494.44 | 565.74 | 20 | 37 | 89 |
| BETAINE | Betaine | 3.025 | 3.596 | 5.3352 | 5.90053 | 8.0909 | 19.3663 | 18 | 36 | 88 |
| N-ACETYLPUTRESCINE | N-Acetylputrescine | 2.272 | 2.472 | 8.8389 | 21.6884 | 7.1087 | 15.0297 | 20 | 36 | 89 |
| GLYCINE | Gly | 65.011 | 74.106 | 244.6 | 523.14 | 184.11 | 365.957 | 20 | 37 | 90 |
| HYPOXANTHINE | Hypoxanthine | 3.410 | 3.105 | 8.2703 | 9.59661 | 7.6871 | 9.55741 | 19 | 35 | 85 |
| LEUCINE | Leu | 10.154 | 12.642 | 40.029 | 111.084 | 32.465 | 79.3207 | 20 | 37 | 90 |
| CROTONIC ACID | Crotonate | 3.972 | 4.571 | 13.987 | 16.6822 | 13.974 | 18.9801 | 13 | 32 | 75 |
| ORNITHINE | Ornithine | 14.858 | 14.943 | 49.144 | 89.3732 | 38.014 | 61.7178 | 20 | 37 | 90 |
| ISOLEUCINE | Ile | 3.739 | 5.159 | 16.472 | 49.1296 | 13.323 | 34.8535 | 20 | 37 | 90 |
| TRYPTOPHAN | Trp | 1.021 | 1.114 | 2.3621 | 3.28089 | 2.3166 | 4.00336 | 16 | 36 | 88 |
| CITRULLINE | Citrulline | 12.637 | 20.416 | 23.047 | 32.1049 | 19.889 | 24.4814 | 20 | 37 | 90 |
| GLUTAMINE | Gln | 17.582 | 20.941 | 52.563 | 125.806 | 48.734 | 118.722 | 20 | 37 | 90 |
| N1-,N8-DIACETYLSPERMIDINE | N1,N8-Diacetylspermidine | 0.097 | 0.090 | 0.2579 | 0.35117 | 0.2501 | 0.38753 | 15 | 32 | 76 |
| PROLINE | Pro | 41.381 | 58.124 | 172.84 | 450.311 | 117.48 | 298.65 | 20 | 37 | 90 |
| 2-OXOISOPENTANOIC ACID | 2-Oxoisopentanoate | 0.783 | 0.465 | 1.3519 | 1.12646 | 1.3467 | 1.33154 | 17 | 34 | 81 |
| GLUTAMIC ACID | Glu | 34.682 | 40.278 | 73.856 | 138.564 | 63.928 | 110.975 | 20 | 37 | 90 |
| 4-METHYLBENZOATE | 4-Methylbenzoate | 13.815 | 15.388 | 34.85 | 50.4018 | 30.807 | 39.5322 | 20 | 35 | 87 |
| 3-(4-HYDROXYPHENYL) | 3-(4- | 5.764 | 6.256 | 18.082 | 23.9319 | 17.157 | 24.1955 | 19 | 35 | 86 |
| PROPIONIC ACID | Hydroxyphenyl)propionate | | | | | | | | | |
| CYSTEIC ACID | Cysteate | 0.140 | 0.129 | 0.4086 | 0.67652 | 0.3194 | 0.5148 | 13 | 27 | 60 |
| AZELAIC ACID | Azelate | 0.037 | 0.129 | 0.0975 | 0.19163 | 0.0913 | 0.15074 | 2 | 15 | 39 |
| RIBULOSE-5-PHOSPHORIC | Ru5P | 3.152 | 2.063 | 4.9381 | 3.74484 | 4.7181 | 3.89669 | 20 | 36 | 88 |
| ACID | | | | | | | | | | |
| PICOLINIC ACID | Pipecolate | 0.622 | 0.813 | 0.9077 | 0.96916 | 0.8528 | 0.87247 | 17 | 34 | 83 |
| PHENYLALANINE | Phe | 14.694 | 12.454 | 28.133 | 35.1399 | 25.384 | 29.0587 | 20 | 37 | 90 |

| | | MANN-WHITNEY TEST | | | | | |
|---|---|---|---|---|---|---|---|
| | | | | INCLUDING | | | |
| | | ONLY BEFORE | | DURING | | | |
| | | TREATMENT | | TREATMENT | | | |
| | | vs HEALTHY | | vs HEALTHY | | | |
| | | SUBJECT | | SUBJECT | | PUBLICLY | |
| NAME OF SUBSTANCE | | P VALUE | Q VALUE | P VALUE | Q VALUE | KNOWN | SIGNIFICANT |
| ALANINE | Ala | 0.00362 | 0.013 | 0.00148 | 0.006 | 7 | 1 |
| VALINE | Val | 0.00381 | 0.014 | 0.00064 | 0.004 | 7 | 1 |
| BUTYRIC ACID | Butanoate | 0.00424 | 0.015 | 0.00065 | 0.004 | | 1 |
| SPERMIDINE | Spermidine | 0.00457 | 0.015 | 0.00821 | 0.02 | 8 | 1 |
| N8-ACETYLSPERMIDINE | N8-Acetylspermidine | 0.00497 | 0.016 | 0.00323 | 0.01 | 8 | 1 |
| ADENINE | Adenine | 0.00514 | 0.016 | 0.01372 | 0.03 | | 1 |
| PUTRESCINE | Putrescine(1,4-Butanediamine) | 0.00536 | 0.017 | 0.00201 | 0.007 | 78 | 1 |
| N6-ACETYLLYSINE | N-epsilon-Acetyllysine | 0.00552 | 0.017 | 0.00453 | 0.013 | | 1 |
| 6-HYDROXYHEXANOIC ACID | 6-Hydroxyhexanoate | 0.00624 | 0.019 | 0.02908 | 0.058 | | 1 |
| PROPIONIC ACID | Propionate | 0.00642 | 0.019 | 0.00394 | 0.012 | | 1 |
| BETAINE | Betaine | 0.00688 | 0.02 | 0.00347 | 0.011 | | 1 |
| N-ACETYLPUTRESCINE | N-Acetylputrescine | 0.00709 | 0.02 | 0.00315 | 0.01 | | 1 |
| GLYCINE | Gly | 0.00727 | 0.02 | 0.00281 | 0.009 | 7 | 1 |
| HYPOXANTHINE | Hypoxanthine | 0.00905 | 0.024 | 0.01064 | 0.025 | | 1 |
| LEUCINE | Leu | 0.00953 | 0.024 | 0.00198 | 0.007 | 7 | 1 |
| CROTONIC ACID | Crotonate | 0.00944 | 0.024 | 0.01306 | 0.03 | | 1 |
| ORNITHINE | Ornithine | 0.01051 | 0.026 | 0.00399 | 0.012 | 78 | 1 |
| ISOLEUCINE | Ile | 0.01076 | 0.026 | 0.00159 | 0.006 | 7 | 1 |
| TRYPTOPHAN | Trp | 0.01237 | 0.03 | 0.00849 | 0.02 | | 1 |
| CITRULLINE | Citrulline | 0.01463 | 0.034 | 0.00566 | 0.015 | | 1 |
| GLUTAMINE | Gln | 0.0164 | 0.038 | 0.0207 | 0.043 | | 1 |
| N1-,N8-DIACETYLSPERMIDINE | N1,N8-Diacetylspermidine | 0.01899 | 0.043 | 0.0671 | 0.118 | 8 | 1 |
| PROLINE | Pro | 0.0201 | 0.045 | 0.0066 | 0.017 | | 1 |
| 2-OXOISOPENTANOIC ACID | 2-Oxoisopentanoate | 0.02087 | 0.046 | 0.0153 | 0.033 | | 1 |
| GLUTAMIC ACID | Glu | 0.02196 | 0.047 | 0.02156 | 0.043 | 7 | 1 |
| 4-METHYLBENZOATE | 4-Methylbenzoate | 0.02558 | 0.054 | 0.00974 | 0.023 | | 1 |
| 3-(4-HYDROXYPHENYL) | 3-(4-Hydroxyphenyl)propionate | 0.02669 | 0.056 | 0.00776 | 0.019 | | 1 |
| PROPIONIC ACID | | | | | | | |
| CYSTEIC ACID | Cysteate | 0.02938 | 0.06 | 0.13727 | 0.209 | | 1 |
| AZELAIC ACID | Azelate | 0.03075 | 0.062 | 0.01386 | 0.03 | | 1 |
| RIBULOSE-5-PHOSPHORIC | Ru5P | 0.03961 | 0.079 | 0.04804 | 0.09 | | 1 |
| ACID | | | | | | | |
| PICOLINIC ACID | Pipecolate | 0.0403 | 0.079 | 0.0329 | 0.063 | | 1 |
| PHENYLALANINE | Phe | 0.04301 | 0.083 | 0.02133 | 0.043 | | 1 |

TABLE 7-3

| | | CONCENTRATION (μM) | | | | | | | DETECTION |
|---|---|---|---|---|---|---|---|---|---|
| | | | | BREAST CANCER | | | | | RATIO (%) |
| | | | | ONLY | | INCLUDING DURING | | | BREAST |
| | | HEALTHY SUBJECT | | BEFORE TREATMENT | | TREATMENT | | | CANCER |
| | | | STANDARD | | STANDARD | | STANDARD | HEALTHY | BEFORE |
| NAME OF SUBSTANCE | | AVERAGE | DEVIATION | AVERAGE | DEVIATION | AVERAGE | DEVIATION | SUBJECT | TREATMENT |
| O-PHOSPHOSERINE | O-Phosphoserine | 0.708 | 0.930 | 1.5125 | 2.1099 | 41.317 | 1.73873 | 14 | 35 |
| MALONIC ACID | Malonate | 0.63945 | 0.329939 | 1.0177 | 0.7514 | 1.055 | 1.06444 | 18 | 36 |
| HEXANOIC ACID | Hexanoate | 13.3189 | 15.28551 | 28.902 | 34.775 | 31.21 | 37.8054 | 20 | 37 |
| 3-PHOSPHOGLYCERIC ACID | 3PG | 3.04736 | 3.338531 | 4.1156 | 4.6142 | 3.806 | 3.88737 | 20 | 36 |
| N-ACETYLGLUTAMIC ACID | N-Acetylglutamate | 0.13999 | 0.127875 | 0.3581 | 0.5293 | 0.304 | 0.42572 | 15 | 30 |
| N-ACETYLGLUCOSAMINE-6- | N-Acetylglucosamine | 0.17916 | 0.254872 | 0.3779 | 0.5735 | 0.355 | 0.66918 | 10 | 25 |
| PHOSPHORIC ACID | 6-phosphate | | | | | | | | |
| 2-OXOBUTYRIC ACID | 2-Oxobutyrate | 3.12964 | 2.243161 | 5.9299 | 5.5128 | 6.043 | 6.8824 | 17 | 33 |
| GLYCYL-GLYCINE | Glu-Glu | 0.22668 | 0.421861 | 0.3931 | 0.5354 | 0.391 | 0.64314 | 6 | 23 |
| LYSINE | Lys | 36.0936 | 38.50907 | 120.17 | 311.97 | 86.9 | 213.649 | 20 | 36 |
| ASPARTIC ACID | Asp | 17.2927 | 16.49612 | 25.784 | 30.243 | 23.1 | 23.215 | 20 | 37 |
| METHIONINE | Met | 1.335 | 1.93562 | 2.2982 | 3.2471 | 2.556 | 4.53023 | 12 | 31 |
| P-HYDROXYPHENYLACETIC ACID | p-Hydroxyphenylacetate | 12.5798 | 24.01714 | 31.185 | 64.355 | 24 | 45.5421 | 12 | 29 |
| AGMATINE | Agmatine | 0.09511 | 0.084621 | 0.1607 | 0.1443 | 0.148 | 0.1435 | 13 | 28 |
| 2-DEOXYRIBOSE 1-PHOSPHORIC ACID | 2-Deoxyribose 1-phosphate | 0.39332 | 0.743072 | 0.8457 | 1.6264 | 0.682 | 1.22119 | 10 | 26 |
| PEPLOMYCIN | PEP | 0.60591 | 0.643731 | 0.9077 | 1.0752 | 0.801 | 0.88615 | 18 | 36 |
| DIHYDROXYACETONEPHOSPHORIC ACID | DHAP | 7.36598 | 3.854232 | 9.1677 | 5.3083 | 8.998 | 5.96742 | 20 | 37 |
| GLYCOLIC ACID | Glycolate | 9.57279 | 3.591728 | 12.61 | 8.9417 | 11.9 | 7.76016 | 20 | 36 |
| HISTAMINE | Histamine | 0.30073 | 0.439731 | 0.8814 | 1.9794 | 0.669 | 1.55616 | 13 | 30 |
| N-ACETYLLEUCINE | N-Acetylleucine | 0.03062 | 0.060836 | 0.1832 | 0.5739 | 0.099 | 0.38111 | 6 | 16 |
| CYTIDINE DISODIUM 5′-MONOPHOSPHATE | CMP | 0.25869 | 0.443067 | 1.0785 | 2.6912 | 0.973 | 2.38666 | 10 | 21 |
| GUANINE | Guanine | 0.71569 | 0.608172 | 1.492 | 1.7957 | 1.271 | 1.4044 | 14 | 32 |
| 4-METHYL-2-OXOPENTANOIC ACID | 4-Methyl-2-oxopentanoate | 1.58389 | 0.943891 | 2.3178 | 2.2787 | 2.289 | 2.07591 | 20 | 37 |
| N-ACETYLASPARTIC ACID | N-Acetylaspartate | 0.68824 | 0.381095 | 1.1339 | 1.3923 | 1.025 | 1.04935 | 20 | 37 |
| TYROSINE | Tyr | 21.4067 | 18.11086 | 35.63 | 43.578 | 31.4 | 38.7827 | 20 | 37 |
| SUCCINIC ACID | Succinate | 19.5264 | 17.89444 | 35.029 | 69.034 | 42.57 | 100.728 | 20 | 37 |
| GLYCEROPHOSPHORIC ACID | Glycerophosphate | 8.39508 | 4.672054 | 9.4613 | 4.481 | 10.02 | 7.03863 | 20 | 37 |
| ALANYL-ALANIHE | Ala-Ala | 0.77481 | 0.970735 | 1.2475 | 1.6193 | 1.115 | 1.47621 | 13 | 27 |
| 1,3-DIAMINOPROPANE | 1,3-Diaminopropane | 1.50535 | 1.791791 | 2.2101 | 2.494 | 1.983 | 2.11129 | 12 | 29 |
| 3-PHENYLPROPIONIC ACID | 3-Phenylpropionate | 9.9887 | 10.32626 | 17.117 | 20.316 | 18.22 | 21.885 | 20 | 31 |
| CIS-ACONITATE | cis-Aconitate | 0.12953 | 0.117343 | 0.2169 | 0.3154 | 0.35 | 0.94761 | 12 | 30 |

| | | DETECTION | MANN-WHITNEY TEST | | | | | |
|---|---|---|---|---|---|---|---|---|
| | | RATIO (%) | ONLY | | INCLUDING DURING | | | |
| | | BREAST CANCER | BEFORE TREATMENT | | TREATMENT | | | |
| | | INCLUDING DURING | vs HEALTHY SUBJECT | | vs HEALTHY SUBJECT | | PUBLICLY | |
| NAME OF SUBSTANCE | | TREATMENT | P VALUE | Q VALUE | P VALUE | Q VALUE | KNOWN | SIGNIFICANT |
| O-PHOSPHOSERINE | O-Phosphoserine | 84 | 0.054 | 0.1023 | 0.047 | 0.08962 | | 1 |
| MALONIC ACID | Malonate | 88 | 0.056 | 0.10535 | 0.017 | 0.03548 | | 1 |
| HEXANOIC ACID | Hexanoate | 89 | 0.062 | 0.11442 | 0.007 | 0.01869 | | 1 |
| 3-PHOSPHOGLYCERIC ACID | 3PG | 89 | 0.066 | 0.1192 | 0.082 | 0.14011 | | 0 |
| N-ACETYLGLUTAMIC ACID | N-Acetylglutamate | 78 | 0.071 | 0.12654 | 0.055 | 0.10058 | | 0 |
| N-ACETYLGLUCOSAMINE-6- | N-Acetylglucosamine | 58 | 0.072 | 0.12654 | 0.145 | 0.21651 | | 0 |
| PHOSPHORIC ACID | 6-phosphate | | | | | | | |
| 2-OXOBUTYRIC ACID | 2-Oxobutyrate | 76 | 0.077 | 0.13429 | 0.102 | 0.17022 | | 0 |
| GLYCYL-GLYCINE | Glu-Glu | 52 | 0.084 | 0.1439 | 0.094 | 0.15826 | | 0 |
| LYSINE | Lys | 89 | 0.088 | 0.14876 | 0.074 | 0.12852 | | 0 |
| ASPARTIC ACID | Asp | 90 | 0.094 | 0.15744 | 0.056 | 0.10156 | 7 | 0 |
| METHIONINE | Met | 75 | 0.097 | 0.15951 | 0.058 | 0.10439 | | 0 |
| P-HYDROXYPHENYLACETIC ACID | p-Hydroxyphenylacetate | 75 | 0.101 | 0.1639 | 0.032 | 0.06241 | | 1 |
| AGMATINE | Agmatine | 66 | 0.106 | 0.16947 | 0.185 | 0.26216 | | 0 |
| 2-DEOXYRIBOSE 1-PHOSPHORIC ACID | 2-Deoxyribose 1-phosphate | 63 | 0.138 | 0.21673 | 0.107 | 0.17574 | | 0 |
| PEPLOMYCIN | PEP | 85 | 0.139 | 0.21673 | 0.162 | 0.23485 | | 0 |
| DIHYDROXYACETONEPHOSPHORIC ACID | DHAP | 90 | 0.146 | 0.22459 | 0.286 | 0.37699 | | 0 |
| GLYCOLIC ACID | Glycolate | 85 | 0.16 | 0.24375 | 0.115 | 0.18355 | | 0 |
| HISTAMINE | Histamine | 77 | 0.169 | 0.25525 | 0.129 | 0.20202 | | 0 |
| N-ACETYLLEUCINE | N-Acetylleucine | 28 | 0.179 | 0.26598 | 0.67 | 0.73379 | | 0 |
| CYTIDINE DISODIUM 5′-MONOPHOSPHATE | CMP | 54 | 0.19 | 0.27997 | 0.202 | 0.28365 | | 0 |
| GUANINE | Guanine | 78 | 0.217 | 0.31541 | 0.144 | 0.21651 | | 0 |
| 4-METHYL-2-OXOPENTANOIC ACID | 4-Methyl-2-oxopentanoate | 90 | 0.222 | 0.31859 | 0.113 | 0.18302 | | 0 |
| N-ACETYLASPARTIC ACID | N-Acetylaspartate | 90 | 0.225 | 0.31979 | 0.162 | 0.23485 | | 0 |
| TYROSINE | Tyr | 90 | 0.229 | 0.32104 | 0.227 | 0.31474 | 7 | 0 |
| SUCCINIC ACID | Succinate | 90 | 0.238 | 0.32735 | 0.24 | 0.32643 | | 0 |
| GLYCEROPHOSPHORIC ACID | Glycerophosphate | 90 | 0.238 | 0.32735 | 0.287 | 0.37699 | | 0 |
| ALANYL-ALANIHE | Ala-Ala | 62 | 0.253 | 0.34319 | 0.378 | 0.47192 | | 0 |
| 1,3-DIAMINOPROPANE | 1,3-Diaminopropane | 71 | 0.275 | 0.3699 | 0.278 | 0.37322 | 8 | 0 |
| 3-PHENYLPROPIONIC ACID | 3-Phenylpropionate | 80 | 0.296 | 0.38057 | 0.126 | 0.19886 | | 0 |
| CIS-ACONITATE | cis-Aconitate | 68 | 0.298 | 0.38057 | 0.134 | 0.20719 | | 0 |

TABLE 7-4

| | | CONCENTRATION (μM) | | | | | | | DETECTION |
|---|---|---|---|---|---|---|---|---|---|
| | | | | BREAST CANCER | | | | | RATIO (%) |
| | | | | ONLY | | INCLUDING DURING | | | BREAST |
| | | HEALTHY SUBJECT | | BEFORE TREATMENT | | TREATMENT | | | CANCER |
| | | | STANDARD | | STANDARD | | STANDARD | HEALTHY | BEFORE |
| NAME OF SUBSTANCE | | AVERAGE | DEVIATION | AVERAGE | DEVIATION | AVERAGE | DEVIATION | SUBJECT | TREATMENT |
| 3-HYDROXYBUTYRIC ACID | 3-Hydroxybutyrate | 6.65264 | 4.679435 | 8.1673 | 5.6145 | 9.119 | 8.0353 | 20 | 37 |
| BENZOIC ACID | Benzoate | 11.5158 | 4.957046 | 9.6971 | 6.7622 | 10.98 | 8.65846 | 18 | 27 |
| GUANOSINE | Guanosinc | 0.18071 | 0.208922 | 0.3316 | 0.4262 | 0.313 | 0.52441 | 10 | 19 |
| 6-PHOSPHOGLUCONIC ACID | 6-Phosphogluconate | 0.45188 | 0.420291 | 0.6064 | 0.5832 | 0.547 | 0.59845 | 16 | 33 |
| URIDYLIC ACID | UMP | 0.12161 | 0.168081 | 0.2179 | 0.2884 | 0.313 | 0.63248 | 8 | 18 |
| ADENOSINE | Adenosine | 0.14107 | 0.164335 | 0.1919 | 0.2021 | 0.163 | 0.18723 | 11 | 22 |
| MUCIC ACID | Mucate | 0.24406 | 0.271429 | 0.1807 | 0.2273 | 0.283 | 0.54345 | 11 | 18 |
| ETHANOLAMINEPHOSPHORIC ACID | Ethanolamine phosphate | 32.346 | 26.12692 | 60.343 | 94.201 | 54.75 | 82.4473 | 18 | 34 |
| ADIPATE | Adipate | 0.49755 | 0.32343 | 0.5903 | 0.531 | 0.562 | 0.39362 | 19 | 35 |
| 2-ISOPROPYLMALATE | 2-Isopropylmalate | 0.25413 | 0.194751 | 0.2663 | 0.1619 | 0.261 | 0.18967 | 20 | 36 |
| PHOSPHORYLCHLORINE | Phosphorylcholine | 5.9658 | 5.079958 | 5.9868 | 6.9568 | 6.471 | 7.37476 | 20 | 31 |
| NICOTINATE | Nicotinate | 1.84972 | 1.398171 | 2.2547 | 2.2648 | 2.606 | 2.66505 | 16 | 23 |
| 1-METHYL-2-PYRROLIDINONE | 1-Methyl-2-pyrrolidinone | 23.0783 | 14.05832 | 30.672 | 33.782 | 27.93 | 28.4191 | 20 | 36 |
| MALIC ACID | Malate | 1.63046 | 0.501086 | 2.4789 | 2.9025 | 2.689 | 2.85035 | 20 | 37 |
| PANTOTHENIC ACID | Pantothenate | 0.15636 | 0.284826 | 0.2627 | 0.5432 | 0.276 | 0.49056 | 7 | 14 |
| N-ACETYLNEURAMINATE | N-Acetylneuraminate | 49.3584 | 36.98463 | 43.496 | 30.441 | 42.27 | 34.3608 | 20 | 37 |
| HISTAMINE | His | 11.4764 | 8.07934 | 16.58 | 20.947 | 15.95 | 19.4737 | 20 | 37 |
| FUMARIC ACID | Fumarate | 0.35582 | 0.233544 | 0.4677 | 0.5926 | 0.57 | 0.68895 | 15 | 30 |
| 2-DEOXYGLUCOSE-6-PHOSPHORIC ACID | 2-Deoxyglucose 6-phosphate | 0.35743 | 0.74316 | 0.4996 | 1.0832 | 0.37 | 0.83301 | 5 | 11 |
| CITRIC ACID | Citrate | 4.60257 | 3.675132 | 4.8562 | 6.0137 | 8.051 | 19.8864 | 20 | 35 |
| 4-ACETYLBUTYRIC ACID | 4-Acetylbutyrate | 0.09595 | 0.160288 | 0.0775 | 0.1312 | 0.072 | 0.12937 | 6 | 11 |
| O-ACETYLCARNITINE | o-Acetylcarnitine | 0.93204 | 0.530378 | 1.3934 | 1.8255 | 1.283 | 1.90322 | 20 | 37 |
| N-ACETYLGLUCOSAMINE-1- | N-Acetylglucosamine | 0.28012 | 0.263447 | 0.265 | 0.3282 | 0.239 | 0.25934 | 17 | 26 |
| PHOSPHORIC ACID | 1-phosphate | | | | | | | | |
| SEDOHEPTULOSE-7-PHOSPHORIC ACID | S7P | 0.49477 | 0.412779 | 0.5007 | 0.3721 | 0.564 | 0.45267 | 16 | 29 |
| HEPTANOIC ACID | Heptanoate | 0.23138 | 0.200911 | 0.2286 | 0.1788 | 0.215 | 0.22231 | 15 | 25 |
| CREATININE | Creatinine | 3.47024 | 1.223945 | 4.3163 | 3.7725 | 4.805 | 8.00241 | 20 | 37 |
| 2,5-DIHYDROXYBENZONATE | 2,5-Dihydroxybenzoate | 0.67706 | 1.220304 | 0.6312 | 1.125 | 0.563 | 0.97834 | 9 | 17 |
| CYTIDINE | Cytidine | 0.2924 | 0.261356 | 0.4567 | 0.6785 | 0.386 | 0.62379 | 14 | 22 |
| ARGININE | Arg | 12.0359 | 6.289379 | 15.501 | 15.934 | 14.71 | 14.7857 | 20 | 37 |
| SYRINGIC ACID | Syringate | 1.13065 | 0.376083 | 1.2108 | 0.7313 | 1.106 | 0.67811 | 20 | 34 |

| | | DETECTION | MANN-WHITNEY TEST | | | | | |
|---|---|---|---|---|---|---|---|---|
| | | RATIO (%) | ONLY | | INCLUDING DURING | | | |
| | | BREAST CANCER | BEFORE TREATMENT | | TREATMENT | | | |
| | | INCLUDING DURING | vs HEALTHY SUBJECT | | vs HEALTHY SUBJECT | | PUBLICLY | |
| NAME OF SUBSTANCE | | TREATMENT | P VALUE | Q VALUE | P VALUE | Q VALUE | KNOWN | SIGNIFICANT |
| 3-HYDROXYBUTYRIC ACID | 3-Hydroxybutyrate | 90 | 0.288 | 0.38057 | 0.179 | 0.2567 | | 0 |
| BENZOIC ACID | Benzoate | 67 | 0.289 | 0.38057 | 0.54 | 0.64014 | | 0 |
| GUANOSINE | Guanosinc | 42 | 0.297 | 0.38057 | 0.675 | 0.73379 | | 0 |
| 6-PHOSPHOGLUCONIC ACID | 6-Phosphogluconate | 77 | 0.319 | 0.40265 | 0.655 | 0.73117 | | 0 |
| URIDYLIC ACID | UMP | 42 | 0.324 | 0.40524 | 0.341 | 0.43454 | | 0 |
| ADENOSINE | Adenosine | 49 | 0.356 | 0.44105 | 0.613 | 0.70354 | | 0 |
| MUCIC ACID | Mucate | 49 | 0.465 | 0.56994 | 0.687 | 0.74006 | | 0 |
| ETHANOLAMINEPHOSPHORIC ACID | Ethanolamine phosphate | 84 | 0.558 | 0.67738 | 0.543 | 0.64014 | | 0 |
| ADIPATE | Adipate | 87 | 0.581 | 0.69792 | 0.319 | 0.41063 | | 0 |
| 2-ISOPROPYLMALATE | 2-Isopropylmalate | 87 | 0.586 | 0.69803 | 0.913 | 0.95163 | | 0 |
| PHOSPHORYLCHLORINE | Phosphorylcholine | 81 | 0.604 | 0.71215 | 0.914 | 0.95163 | | 0 |
| NICOTINATE | Nicotinate | 63 | 0.628 | 0.73378 | 0.386 | 0.47816 | | 0 |
| 1-METHYL-2-PYRROLIDINONE | 1-Methyl-2-pyrrolidinone | 87 | 0.64 | 0.74026 | 0.978 | 0.98625 | | 0 |
| MALIC ACID | Malate | 90 | 0.651 | 0.74644 | 0.23 | 0.31652 | | 0 |
| PANTOTHENIC ACID | Pantothenate | 42 | 0.678 | 0.76994 | 0.313 | 0.40777 | | 0 |
| N-ACETYLNEURAMINATE | N-Acetylneuraminate | 90 | 0.744 | 0.80908 | 0.464 | 0.56853 | | 0 |
| HISTAMINE | His | 90 | 0.738 | 0.80908 | 0.551 | 0.64092 | | 0 |
| FUMARIC ACID | Fumarate | 72 | 0.743 | 0.80908 | 0.554 | 0.64092 | | 0 |
| 2-DEOXYGLUCOSE-6-PHOSPHORIC ACID | 2-Deoxyglucose 6-phosphate | 24 | 0.744 | 0.80908 | 0.972 | 0.98625 | | 0 |
| CITRIC ACID | Citrate | 86 | 0.732 | 0.80908 | 1 | 1 | | 0 |
| 4-ACETYLBUTYRIC ACID | 4-Acetylbutyrate | 25 | 0.772 | 0.82508 | 0.625 | 0.71055 | | 0 |
| O-ACETYLCARNITINE | o-Acetylcarnitine | 89 | 0.77 | 0.82508 | 0.96 | 0.98625 | | 0 |
| N-ACETYLGLUCOSAMINE-1- | N-Acetylglucosamine | 63 | 0.781 | 0.82726 | 0.675 | 0.73379 | | 0 |
| PHOSPHORIC ACID | 1-phosphate | | | | | | | |
| SEDOHEPTULOSE-7-PHOSPHORIC ACID | S7P | 72 | 0.814 | 0.85505 | 0.518 | 0.62283 | | 0 |
| HEPTANOIC ACID | Heptanoate | 56 | 0.826 | 0.85996 | 0.654 | 0.73117 | | 0 |
| CREATININE | Creatinine | 90 | 0.867 | 0.89576 | 0.877 | 0.92877 | | 0 |
| 2,5-DIHYDROXYBENZONATE | 2,5-Dihydroxybenzoate | 38 | 0.898 | 0.92041 | 0.767 | 0.81912 | | 0 |
| CYTIDINE | Cytidine | 49 | 0.959 | 0.96668 | 0.508 | 0.6165 | | 0 |
| ARGININE | Arg | 90 | 0.953 | 0.96668 | 0.972 | 0.98625 | | 0 |
| SYRINGIC ACID | Syringate | 82 | 0.993 | 0.99331 | 0.374 | 0.47183 | | 0 |

A substance for which “7” or “8” is indicated in the column labeled “Publicly Known” is a known substance disclosed in Non-Patent Literature 7 or 8.

Next, a network diagram in which a line is drawn between metabolites exhibiting a correlation between metabolites in the patients with breast cancer before initiation of treatment (37 cases) and metabolites in the healthy subjects (20 cases) of R²>0.92 shown in FIG. 10 is shown. Among the substances, a substance that formed bonding lines to many substances and could be detected in all of the samples, or glutamine (Gln), was selected as a concentration-correcting substance. In the drawing, the substance is circled.

Substances in which a relative concentration exhibits a statistical significant difference (p<0.05 in the Mann-Whitney test) between the patients with breast cancer and the healthy subjects are shown in Tables 8-1, 8-2, 8-3, and 8-4. In calculating the relative concentration, the concentration of each substance was divided by the concentration of glutamine, and the value was expressed with no units.

At that time, many of the metabolites included in saliva were measured. In order to calculate the significant difference of each substance, independent statistics (for example, Mann-Whitney test) needs to be repeated. When the test is repeated at a level of significance a of 0.05, null hypothesis that is accidentally dismissed is increased. Therefore, the P value was corrected by the false discovery rate (FDR) method (Storey, J. D., & Tibshirani, R. (2003). Statistical significance for genomewide studies. Proceedings of the National academy of Sciences of the United States of America, 100, 9440-9445), and a Q value was calculated. For example, when the Q value is 0.5, true null hypothesis occupies a half of the null hypothesis that is dismissed at P<0.05.

TABLE 8-1

| | | RELATIVE CONCENTRATION (NO UNIT) | | | | | | | DETECTION | |
|---|---|---|---|---|---|---|---|---|---|---|
| | | | | BREAST CANCER | | | | | RATIO (%) | |
| | | | | ONLY BEFORE | | INCLUDING DURING | | | BREAST CANCER | |
| | | HEALTHY SUBJECT | | TREATMENT | | TREATMENT | | | | INCLUDING |
| | | | STANDARD | | STANDARD | | STANDARD | HEALTHY | BEFORE | DURING |
| NAME OF SUBSTANCE | | AVERAGE | DEVIATION | AVERAGE | DEVIATION | AVERAGE | DEVIATION | SUBJECT | TREATMENT | TREATMENT |
| CHOLINE | Choline | 0.535 | 0.365 | 0.6695 | 0.39647 | 0.7124 | 0.392 | 20 | 37 | 90 |
| 2-HYDROXYBUTYRIC ACID | 2-Hydroxybutyrate | 0.126 | 0.110 | 0.1339 | 0.14048 | 0.1438 | 0.132294 | 18 | 37 | 90 |
| β-ALANINE | beta-Ala | 0.102 | 0.081 | 0.1503 | 0.07955 | 0.1676 | 0.118533 | 17 | 36 | 87 |
| 3-METHYLHISDINE | 3-Methylhistidine | 0.007 | 0.016 | 0.0294 | 0.03108 | 0.0331 | 0.038386 | 6 | 28 | 70 |
| α-AMINOBUTYRIC ACID | 2AB | 0.057 | 0.043 | 0.0989 | 0.05536 | 0.1191 | 0.102119 | 16 | 35 | 86 |
| CADAVERINE | Cadaverine | 0.602 | 0.539 | 1.2257 | 0.86809 | 1.6484 | 1.741134 | 19 | 37 | 89 |
| N-ACETYL-β-ALANINE | N-Acetyl-beta-alanine | 0.021 | 0.031 | 0.0409 | 0.03495 | 0.0424 | 0.033048 | 9 | 32 | 76 |
| ISETHIONIC ACID | Isethionate | 0.017 | 0.016 | 0.0162 | 0.01528 | 0.0178 | 0.025157 | 14 | 32 | 77 |
| N-ACETYLPHENYLALANINE | N-Acetylphenylalanine | 0.007 | 0.011 | 0.0003 | 0.00108 | 0.0007 | 0.002282 | 10 | 3 | 10 |
| TRIMETHYLLYSINE | N6,N6,N6-Trimethyllysine | 0.001 | 0.004 | 0.0071 | 0.00828 | 0.0075 | 0.008624 | 3 | 25 | 57 |
| α-AMINOADIPIC ACID | alpha-Aminoadipate | 0.073 | 0.056 | 0.089 | 0.05484 | 0.095 | 0.065314 | 17 | 34 | 83 |
| SPERMINE | Spermine | 0.004 | 0.007 | 0.0207 | 0.02522 | 0.0199 | 0.030295 | 6 | 28 | 60 |
| N1-ACETYLSPERMIDINE | N1-Acetylspermidine | 0.025 | 0.026 | 0.0386 | 0.0295 | 0.0425 | 0.032472 | 15 | 35 | 87 |
| CREATINE | Creatine | 1.034 | 0.560 | 0.9723 | 0.44164 | 1.0219 | 0.578194 | 20 | 37 | 90 |
| γ-BUTYROBETAINE | gamma-Butyrobetaine | 0.154 | 0.089 | 0.2152 | 0.14508 | 0.232 | 0.161565 | 20 | 36 | 89 |
| SARCOSINE | Sarcosine | 0.416 | 0.199 | 0.4475 | 0.25949 | 0.4453 | 0.284517 | 20 | 37 | 90 |
| PYRUVIC ACID | Pyruvate | 3.853 | 2.269 | 4.1335 | 2.94749 | 4.6006 | 3.016964 | 20 | 37 | 90 |
| UROCANIC ACID | Urocanate | 0.070 | 0.083 | 0.1517 | 0.19424 | 0.1848 | 0.27465 | 13 | 32 | 82 |
| PIPERIDINE | Piperidine | 0.006 | 0.011 | 0.0159 | 0.02094 | 0.0348 | 0.107037 | 7 | 25 | 67 |
| SERINE | Ser | 0.708 | 0.279 | 0.9755 | 0.86402 | 1.0627 | 1.0582 | 20 | 37 | 90 |
| HOMOVANILLIC ACID | Homovanillate | 0.155 | 0.073 | 0.143 | 0.07656 | 0.1857 | 0.14732 | 20 | 37 | 88 |
| 5-OXOPROLINE | 5-Oxoproline | 0.568 | 0.887 | 0.556 | 0.62212 | 0.7212 | 1.230777 | 20 | 37 | 90 |
| GABA | GABA | 0.100 | 0.046 | 0.1004 | 0.06762 | 0.1752 | 0.460201 | 20 | 36 | 89 |
| 5-AMINOVALERIC ACID | 5-Aminovalerate | 13.413 | 11.107 | 23.718 | 19.2024 | 27.708 | 23.34253 | 20 | 37 | 89 |
| TRIMETHYLAMINE-N-OXIDE | Trimethylamine N-oxide | 0.014 | 0.030 | 0.0244 | 0.03508 | 0.0222 | 0.031137 | 4 | 24 | 61 |
| 2-HYDROXYVALERIC ACID | 2-Hydroxypentanoate | 0.503 | 0.284 | 0.5272 | 0.35447 | 0.6226 | 0.481419 | 20 | 37 | 90 |
| CARNITINE | Carnitine | 0.064 | 0.028 | 0.0598 | 0.0358 | 0.0589 | 0.030769 | 20 | 37 | 90 |
| ISOPROPANOLAMINE | Isopropanolamine | 0.040 | 0.053 | 0.061 | 0.07286 | 0.0734 | 0.084822 | 12 | 31 | 77 |
| THREONINE | Thr | 0.286 | 0.085 | 0.3742 | 0.24147 | 0.4102 | 0.288762 | 20 | 37 | 90 |
| HYPOTAURINE | Hypotaurine | 0.007 | 0.020 | 0.0487 | 0.06827 | 0.05 | 0.072847 | 2 | 19 | 42 |
| LACTIC ACID | Lactate | 13.476 | 7.556 | 13.847 | 10.6027 | 15.565 | 17.77329 | 20 | 37 | 90 |
| 2-HYDROXY-4-METHYL- | 2-Hydroxy-4-methylpentanoate | 0.144 | 0.093 | 0.1792 | 0.1286 | 0.2112 | 0.174675 | 20 | 36 | 89 |
| PENTANOIC ACID | | | | | | | | | | |
| HYDROXYPROLINE | Hydroxyproline | 0.025 | 0.041 | 0.048 | 0.08291 | 0.0459 | 0.059641 | 9 | 31 | 75 |
| ALANINE | Ala | 1.408 | 0.577 | 1.7516 | 0.78559 | 2.0262 | 1.3785 | 20 | 37 | 90 |

| | | MANN-WHITNEY TEST | | | | | |
|---|---|---|---|---|---|---|---|
| | | ONLY | | INCLUDING | | | |
| | | BEFORE | | DURING | | | |
| | | TREATMENT | | TREATMENT | | | |
| | | vs HEALTHY | | vs HEALTHY | | | |
| | | SUBJECT | | SUBJECT | | PUBLICLY | |
| NAME OF SUBSTANCE | | P VALUE | Q VALUE | P VALUE | Q VALUE | KNOWN | SIGNIFICANT |
| CHOLINE | Choline | 0.20875 | 0.42434 | 0.042705 | 0.129157 | | 1 |
| 2-HYDROXYBUTYRIC ACID | 2-Hydroxybutyrate | 0.7955 | 0.90497 | 0.695525 | 0.821382 | | 0 |
| β-ALANINE | beta-Ala | 0.02613 | 0.12032 | 0.010775 | 0.049363 | | 1 |
| 3-METHYLHISDINE | 3-Methylhistidine | 0.00022 | 0.00989 | 2.52E−05 | 0.001565 | | 1 |
| α-AMINOBUTYRIC ACID | 2AB | 0.00841 | 0.0652 | 0.001245 | 0.02205 | | 1 |
| CADAVERINE | Cadaverine | 0.00496 | 0.05 | 0.001936 | 0.025253 | 78 | 1 |
| N-ACETYL-β-ALANINE | N-Acetyl-beta-alanine | 0.01176 | 0.07673 | 0.003595 | 0.03184 | | 1 |
| ISETHIONIC ACID | Isethionate | 0.88658 | 0.93166 | 0.891843 | 0.929316 | | 0 |
| N-ACETYLPHENYLALANINE | N-Acetylphenylalanine | 0.00017 | 0.00989 | 2.41E−05 | 0.001565 | | 1 |
| TRIMETHYLLYSINE | N6,N6,N6-Trimethyllysine | 0.00045 | 0.01366 | 0.000233 | 0.009643 | | 1 |
| α-AMINOADIPIC ACID | alpha-Aminoadipate | 0.2996 | 0.53842 | 0.182378 | 0.337535 | | 0 |
| SPERMINE | Spermine | 0.00077 | 0.01585 | 0.001713 | 0.025253 | 8 | 1 |
| N1-ACETYLSPERMIDINE | N1-Acetylspermidine | 0.07745 | 0.20434 | 0.019641 | 0.073801 | 8 | 1 |
| CREATINE | Creatine | 0.8749 | 0.92725 | 0.741877 | 0.824197 | | 0 |
| γ-BUTYROBETAINE | gamma-Butyrobetaine | 0.15403 | 0.34726 | 0.076587 | 0.202061 | | 0 |
| SARCOSINE | Sarcosine | 0.78449 | 0.90322 | 0.990725 | 0.990725 | | 0 |
| PYRUVIC ACID | Pyruvate | 1 | 1 | 0.459233 | 0.618967 | | 0 |
| UROCANIC ACID | Urocanate | 0.05766 | 0.17023 | 0.004244 | 0.035084 | | 1 |
| PIPERIDINE | Piperidine | 0.02259 | 0.11672 | 0.001153 | 0.02205 | | 1 |
| SERINE | Ser | 0.39241 | 0.63739 | 0.152761 | 0.315079 | | 0 |
| HOMOVANILLIC ACID | Homovanillate | 0.63652 | 0.81967 | 0.97836 | 0.986315 | | 0 |
| 5-OXOPROLINE | 5-Oxoproline | 0.81009 | 0.91124 | 0.249793 | 0.407335 | | 0 |
| GABA | GABA | 0.66039 | 0.8356 | 0.650285 | 0.77534 | | 0 |
| 5-AMINOVALERIC ACID | 5-Aminovalerate | 0.03978 | 0.13186 | 0.008131 | 0.049363 | | 1 |
| TRIMETHYLAMINE-N-OXIDE | Trimethylamine N-oxide | 0.01743 | 0.09637 | 0.006256 | 0.0431 | | 1 |
| 2-HYDROXYVALERIC ACID | 2-Hydroxypentanoate | 0.91422 | 0.95264 | 0.579501 | 0.717992 | | 0 |
| CARNITINE | Carnitine | 0.32275 | 0.56367 | 0.29367 | 0.444087 | | 0 |
| ISOPROPANOLAMINE | Isopropanolamine | 0.1805 | 0.37303 | 0.038945 | 0.120731 | | 1 |
| THREONINE | Thr | 0.40171 | 0.63862 | 0.086054 | 0.213453 | 7 | 0 |
| HYPOTAURINE | Hypotaurine | 0.00265 | 0.04689 | 0.002568 | 0.028374 | | 1 |
| LACTIC ACID | Lactate | 0.75912 | 0.90322 | 0.736028 | 0.824197 | | 0 |
| 2-HYDROXY-4-METHYL- | 2-Hydroxy-4-methylpentanoate | 0.48063 | 0.70115 | 0.168946 | 0.331163 | | 0 |
| PENTANOIC ACID | | | | | | | |
| HYDROXYPROLINE | Hydroxyproline | 0.04115 | 0.13186 | 0.010739 | 0.049363 | | 1 |
| ALANINE | Ala | 0.08353 | 0.2115 | 0.012718 | 0.053624 | 7 | 1 |

TABLE 8-2

| | | RELATIVE CONCENTRATION (NO UNIT) | | | | | | | DETECTION | |
|---|---|---|---|---|---|---|---|---|---|---|
| | | | | BREAST CANCER | | | | | RATIO (%) | |
| | | | | ONLY BEFORE | | INCLUDING DURING | | | BREAST CANCER | |
| | | HEALTHY SUBJECT | | TREATMENT | | TREATMENT | | | | INCLUDING |
| | | | STANDARD | | STANDARD | | STANDARD | HEALTHY | BEFORE | DURING |
| NAME OF SUBSTANCE | | AVERAGE | DEVIATION | AVERAGE | DEVIATION | AVERAGE | DEVIATION | SUBJECT | TREATMENT | TREATMENT |
| VALINE | Val | 0.655 | 0.505 | 0.92 | 0.4944 | 1.085 | 0.7477 | 20 | 37 | 90 |
| BUTYRIC ACID | Butanoate | 5.412 | 4.454 | 8.34 | 9.2551 | 10.66 | 12.991 | 20 | 37 | 89 |
| SPERMIDINE | Spermidine | 0.106 | 0.082 | 0.12 | 0.0928 | 0.12 | 0.0849 | 20 | 36 | 88 |
| N8-ACETYLSPERMIDINE | N8-Acetylspermidine | 0.001 | 0.001 | 0 | 0.0025 | 0.002 | 0.003 | 3 | 20 | 49 |
| ADENINE | Adenine | 0.055 | 0.046 | 0.05 | 0.0394 | 0.058 | 0.0424 | 16 | 34 | 81 |
| PUTRESCINE | Putrescine(1,4-Butanediamine) | 2.585 | 2.076 | 3.86 | 2.4115 | 4.412 | 3.3521 | 20 | 37 | 89 |
| N6-ACETYLLYSINE | N-epsilon-Acetyllysine | 0.004 | 0.009 | 0.01 | 0.0113 | 0.01 | 0.0126 | 4 | 22 | 51 |
| 6-HYDROXYHEXANOIC | 6-Hydroxyhexanoate | 0.103 | 0.142 | 0.01 | 0.0439 | 0.027 | 0.0732 | 9 | 5 | 19 |
| ACID | | | | | | | | | | |
| PROPIONIC ACID | Propionate | 15.110 | 13.467 | 19.6 | 16.982 | 21.9 | 19.007 | 20 | 37 | 89 |
| BETAINE | Betaine | 0.235 | 0.161 | 0.21 | 0.1575 | 0.367 | 0.9139 | 18 | 36 | 88 |
| N-ACETYLPUTRESCINE | N-Acetylputrescine | 0.140 | 0.098 | 0.2 | 0.1397 | 0.227 | 0.1674 | 20 | 36 | 89 |
| GLYCINE | Gly | 3.852 | 1.481 | 4.95 | 2.8102 | 5.317 | 3.6606 | 20 | 37 | 90 |
| HYPOXANTHINE | Hypoxanthine | 0.255 | 0.144 | 0.27 | 0.2042 | 0.279 | 0.2112 | 19 | 35 | 85 |
| LEUCINE | Leu | 0.551 | 0.355 | 0.69 | 0.3443 | 0.823 | 0.5284 | 20 | 37 | 90 |
| CROTONIC ACID | Crotonate | 0.323 | 0.408 | 0.77 | 1.2057 | 0.785 | 1.1358 | 13 | 32 | 75 |
| ORNITHINE | Ornithine | 1.004 | 0.517 | 1.34 | 1.0714 | 1.364 | 1.0031 | 20 | 37 | 90 |
| ISOLEUCINE | Ile | 0.187 | 0.134 | 0.25 | 0.1419 | 0.304 | 0.2163 | 20 | 37 | 90 |
| TRYPTOPHAN | Trp | 0.063 | 0.054 | 0.07 | 0.0479 | 0.08 | 0.0572 | 16 | 36 | 88 |
| CITRULLINE | Citrulline | 0.631 | 0.477 | 0.66 | 0.4836 | 0.711 | 0.4288 | 20 | 37 | 90 |
| GLUTAMINE | Gln | 1.000 | 0.000 | 1 | 0 | 1 | 0 | 20 | 37 | 90 |
| N1-,N8-DIACETYLSPERMIDINE | N1,N8-Diacetylspermidine | 0.006 | 0.005 | 0.01 | 0.0074 | 0.008 | 0.008 | 15 | 32 | 76 |
| PROLINE | Pro | 2.119 | 0.813 | 3.11 | 3.2776 | 3.232 | 3.1517 | 20 | 37 | 90 |
| 2-OXOISOPENTANOIC | 2-Oxoisopentanoate | 0.082 | 0.058 | 0.06 | 0.0537 | 0.066 | 0.0518 | 17 | 34 | 81 |
| ACID | | | | | | | | | | |
| GLUTAMIC ACID | Glu | 2.005 | 0.996 | 1.91 | 1.0563 | 2.005 | 1.1163 | 20 | 37 | 90 |
| 4-METHYLBENZOATE | 4-Methylbenzoate | 1.109 | 1.178 | 1.19 | 1.201 | 1.376 | 1.3512 | 20 | 35 | 87 |
| 3-(4-HYDROXYPHENYL) | 3-(4-Hydroxyphenyl)propionate | 0.456 | 0.466 | 0.67 | 0.8923 | 0.711 | 0.7999 | 19 | 35 | 86 |
| PROPIONIC ACID | | | | | | | | | | |
| CYSTEIC ACID | Cysteate | 0.014 | 0.014 | 0.01 | 0.0115 | 0.011 | 0.0118 | 13 | 27 | 60 |
| AZELAIC ACID | Azelate | 0.003 | 0.010 | 0 | 0.0096 | 0.006 | 0.0124 | 2 | 15 | 39 |
| RIBULOSE-5-PHOSPHORIC | Ru5P | 0.292 | 0.160 | 0.23 | 0.1685 | 0.245 | 0.1554 | 20 | 36 | 88 |
| ACID | | | | | | | | | | |
| PICOLINIC ACID | Pipecolate | 0.060 | 0.109 | 0.03 | 0.0234 | 0.037 | 0.0289 | 17 | 34 | 83 |
| PHENYLALANINE | Phe | 1.085 | 0.645 | 0.88 | 0.4717 | 1.039 | 0.652 | 20 | 37 | 90 |

| | | MANN-WHITNEY TEST | | | | | |
|---|---|---|---|---|---|---|---|
| | | ONLY | | INCLUDING | | | |
| | | BEFORE | | DURING | | | |
| | | TREATMENT | | TREATMENT | | | |
| | | vs HEALTHY | | vs HEALTHY | | | |
| | | SUBJECT | | SUBJECT | | PUBLICLY | |
| NAME OF SUBSTANCE | | P VALUE | Q VALUE | P VALUE | Q VALUE | KNOWN | SIGNIFICANT |
| VALINE | Val | 0.01043 | 0.0719 | 0.0009 | 0.022 | 7 | 1 |
| BUTYRIC ACID | Butanoate | 0.53387 | 0.7523 | 0.10781 | 0.2476 | | 0 |
| SPERMIDINE | Spermidine | 0.54486 | 0.7532 | 0.43606 | 0.6075 | 8 | 0 |
| N8-ACETYLSPERMIDINE | N8-Acetylspermidine | 0.00706 | 0.0584 | 0.00275 | 0.0284 | 8 | 1 |
| ADENINE | Adenine | 1 | 1 | 0.72416 | 0.8242 | | 0 |
| PUTRESCINE | Putrescine(1,4-Butanediamine) | 0.03978 | 0.1319 | 0.01115 | 0.0494 | 78 | 1 |
| N6-ACETYLLYSINE | N-epsilon-Acetyllysine | 0.01409 | 0.0832 | 0.00812 | 0.0494 | | 1 |
| 6-HYDROXYHEXANOIC | 6-Hydroxyhexanoate | 0.00361 | 0.05 | 0.00982 | 0.0494 | | 1 |
| ACID | | | | | | | |
| PROPIONIC ACID | Propionate | 0.3653 | 0.6205 | 0.12208 | 0.2703 | | 0 |
| BETAINE | Betaine | 0.4568 | 0.6824 | 0.86766 | 0.9209 | | 0 |
| N-ACETYLPUTRESCINE | N-Acetylputrescine | 0.11859 | 0.2723 | 0.02693 | 0.092 | | 1 |
| GLYCINE | Gly | 0.17447 | 0.373 | 0.04598 | 0.1357 | 7 | 1 |
| HYPOXANTHINE | Hypoxanthine | 0.83443 | 0.9112 | 0.95673 | 0.9724 | | 0 |
| LEUCINE | Leu | 0.05291 | 0.164 | 0.00521 | 0.0404 | 7 | 1 |
| CROTONIC ACID | Crotonate | 0.08358 | 0.2115 | 0.03365 | 0.107 | | 1 |
| ORNITHINE | Ornithine | 0.48063 | 0.7012 | 0.23726 | 0.3974 | 78 | 0 |
| ISOLEUCINE | Ile | 0.04147 | 0.1319 | 0.00327 | 0.0312 | 7 | 1 |
| TRYPTOPHAN | Trp | 0.4367 | 0.6704 | 0.16891 | 0.3312 | | 0 |
| CITRULLINE | Citrulline | 0.69684 | 0.8555 | 0.20512 | 0.3634 | | 0 |
| GLUTAMINE | Gln | NA | NA | NA | NA | | 0 |
| N1-,N8-DIACETYLSPERMIDINE | N1,N8-Diacetylspermidine | 0.56865 | 0.7664 | 0.43032 | 0.6064 | 8 | 0 |
| PROLINE | Pro | 0.77177 | 0.9032 | 0.253 | 0.4073 | | 0 |
| 2-OXOISOPENTANOIC | 2-Oxoisopentanoate | 0.29187 | 0.535 | 0.22048 | 0.3797 | | 0 |
| ACID | | | | | | | |
| GLUTAMIC ACID | Glu | 0.68461 | 0.8518 | 0.86767 | 0.9209 | 7 | 0 |
| 4-METHYLBENZOATE | 4-Methylbenzoate | 0.71922 | 0.8743 | 0.34639 | 0.5053 | | 0 |
| 3-(4-HYDROXYPHENYL) | 3-(4-Hydroxyphenyl)propionate | 0.55276 | 0.7532 | 0.17378 | 0.3312 | | 0 |
| PROPIONIC ACID | | | | | | | |
| CYSTEIC ACID | Cysteate | 0.64119 | 0.8197 | 0.5094 | 0.6512 | | 0 |
| AZELAIC ACID | Azelate | 0.03779 | 0.1319 | 0.0165 | 0.0639 | | 1 |
| RIBULOSE-5-PHOSPHORIC | Ru5P | 0.15896 | 0.352 | 0.25623 | 0.4073 | | 0 |
| ACID | | | | | | | |
| PICOLINIC ACID | Pipecolate | 0.41234 | 0.6472 | 0.72137 | 0.8242 | | 0 |
| PHENYLALANINE | Phe | 0.24078 | 0.4665 | 0.58482 | 0.718 | | 0 |

TABLE 8-3

| | | RELATIVE CONCENTRATION (NO UNIT) | | | | | | | DETECTION |
|---|---|---|---|---|---|---|---|---|---|
| | | | | BREAST CANCER | | | | | RATIO (%) |
| | | | | ONLY BEFORE | | INCLUDING DURING | | | BREAST |
| | | HEALTHY SUBJECT | | TREATMENT | | TREATMENT | | | CANCER |
| | | | STANDARD | | STANDARD | | STANDARD | HEALTHY | BEFORE |
| NAME OF SUBSTANCE | | AVERAGE | DEVIATION | AVERAGE | DEVIATION | AVERAGE | DEVIATION | SUBJECT | TREATMENT |
| O-PHOSPHOSERINE | O-Phosphoserine | 0.045 | 0.060 | 0.045 | 0.0378 | 0.0488 | 0.03918 | 14 | 35 |
| MALONIC ACID | Malonate | 0.0721 | 0.05367 | 0.046 | 0.0335 | 0.0578 | 0.04882 | 18 | 36 |
| HEXANOIC ACID | Hexanoate | 1.1716 | 1.30695 | 1.794 | 2.2027 | 2.0692 | 2.44959 | 20 | 37 |
| 3-PHOSPHOGLYCERIC ACID | 3PG | 0.2068 | 0.12486 | 0.164 | 0.1395 | 0.1728 | 0.13155 | 20 | 36 |
| N-ACETYLGLUTAMIC ACID | N-Acetylglutamate | 0.0101 | 0.00855 | 0.01 | 0.008 | 0.0107 | 0.00868 | 15 | 30 |
| N-ACETYLGLUCOSAMINE-6-PHOSPHORIC ACID | N-Acetylglucosamine 6-phosphate | 0.0135 | 0.01997 | 0.012 | 0.0129 | 0.0121 | 0.01325 | 10 | 25 |
| 2-OXOBUTYRIC ACID | 2-Oxobutyrate | 0.3398 | 0.32933 | 0.288 | 0.3022 | 0.2977 | 0.31856 | 17 | 33 |
| GLYCYL-GLYCINE | Glu-Glu | 0.007 | 0.01345 | 0.011 | 0.0141 | 0.0119 | 0.01543 | 6 | 23 |
| LYSINE | Lys | 2.37 | 1.17409 | 2.109 | 1.2091 | 2.2092 | 1.19698 | 20 | 36 |
| ASPARTIC ACID | Asp | 1.2001 | 0.49598 | 0.926 | 0.6067 | 1.0226 | 0.60552 | 20 | 37 |
| METHIONINE | Met | 0.063 | 0.07532 | 0.073 | 0.073 | 0.0827 | 0.10958 | 12 | 31 |
| P-HYDROXYPHENYLACETIC ACID | p-Hydroxyphenylacetate | 0.8015 | 1.44237 | 0.77 | 0.9516 | 0.8143 | 0.8509 | 12 | 29 |
| AGMATINE | Agmatine | 0.0072 | 0.00696 | 0.007 | 0.0084 | 0.0072 | 0.00815 | 13 | 28 |
| 2-DEOXYRIBOSE 1-PHOSPHORIC ACID | 2-Deoxyribose 1-phosphate | 0.0246 | 0.04973 | 0.022 | 0.0305 | 0.0235 | 0.03225 | 10 | 26 |
| PEPLOMYCIN | PEP | 0.0429 | 0.03478 | 0.03 | 0.0222 | 0.0328 | 0.02543 | 18 | 36 |
| DIHYDROXYACETONEPHOSPHORIC ACID | DHAP | 0.6886 | 0.3374 | 0.445 | 0.3238 | 0.5023 | 0.36005 | 20 | 37 |
| GLYCOLIC ACID | Glycolate | 1.0617 | 0.75961 | 0.677 | 0.6006 | 0.7289 | 0.69866 | 20 | 36 |
| HISTAMINE | Histamine | 0.0236 | 0.04078 | 0.02 | 0.035 | 0.0194 | 0.02611 | 13 | 30 |
| N-ACETYLLEUCINE | N-Acetylleucine | 0.0018 | 0.0034 | 0.002 | 0.0029 | 0.0015 | 0.00294 | 6 | 16 |
| CYTIDINE DISODIUM 5′-MONOPHOSPHATE | CMP | 0.0228 | 0.0348 | 0.031 | 0.0476 | 0.0282 | 0.04103 | 10 | 21 |
| GUANINE | Guanine | 0.0706 | 0.07096 | 0.061 | 0.0505 | 0.0678 | 0.0594 | 14 | 32 |
| 4-METHYL-2-OXOPENTANOIC ACID | 4-Methyl-2-oxopentanoate | 0.1425 | 0.07182 | 0.102 | 0.075 | 0.1129 | 0.07547 | 20 | 37 |
| N-ACETYLASPARTIC ACID | N-Acetylaspartate | 0.0622 | 0.03537 | 0.046 | 0.0434 | 0.0558 | 0.05725 | 20 | 37 |
| TYROSINE | Tyr | 1.5902 | 0.72068 | 1.093 | 0.6385 | 1.165 | 0.61484 | 20 | 37 |
| SUCCINIC ACID | Succinate | 1.61 | 1.54845 | 1.123 | 0.7861 | 1.6525 | 2.61266 | 20 | 37 |
| GLYCEROPHOSPHORIC ACID | Glycerophosphate | 0.7889 | 0.54774 | 0.522 | 0.4592 | 0.6148 | 0.53233 | 20 | 37 |
| ALANYL-ALANINE | Ala-Ala | 0.0498 | 0.0542 | 0.035 | 0.0332 | 0.0388 | 0.03904 | 13 | 27 |
| 1,3-DIAMINOPROPANE | 1,3-Diaminopropane | 0.1091 | 0.12897 | 0.096 | 0.107 | 0.097 | 0.10061 | 12 | 29 |
| 3-PHENYLPROPIONIC ACID | 3-Phenylpropionate | 0.9437 | 1.09581 | 1.178 | 1.659 | 1.395 | 1.91697 | 20 | 31 |
| CIS-ACONITATE | cis-Aconitate | 0.0175 | 0.01899 | 0.013 | 0.0169 | 0.0212 | 0.06004 | 12 | 30 |

| | | DETECTION | MANN-WHITNEY TEST | | | | | |
|---|---|---|---|---|---|---|---|---|
| | | RATIO (%) | ONLY BEFORE | | INCLUDING | | | |
| | | BREAST | TREATMENT | | DURING | | | |
| | | CANCER | vs HEALTHY | | TREATMENT vs | | | |
| | | INCLUDING DURING | SUBJECT | | HEALTHY SUBJECT | | PUBLICLY | |
| NAME OF SUBSTANCE | | TREATMENT | P VALUE | Q VALUE | P VALUE | Q VALUE | KNOWN | SIGNIFICANT |
| O-PHOSPHOSERINE | O-Phosphoserine | 84 | 0.2802 | 0.52635 | 0.17599 | 0.3312 | | 0 |
| MALONIC ACID | Malonate | 88 | 0.0961 | 0.23376 | 0.24034 | 0.3974 | | 0 |
| HEXANOIC ACID | Hexanoate | 89 | 0.7845 | 0.90322 | 0.17626 | 0.3312 | | 0 |
| 3-PHOSPHOGLYCERIC ACID | 3PG | 89 | 0.0898 | 0.22266 | 0.155 | 0.3151 | | 0 |
| N-ACETYLGLUTAMIC ACID | N-Acetylglutamate | 78 | 0.9732 | 0.99073 | 0.74142 | 0.8242 | | 0 |
| N-ACETYLGLUCOSAMINE-6-PHOSPHORIC ACID | N-Acetylglucosamine 6-phosphate | 58 | 0.5296 | 0.75227 | 0.57941 | 0.718 | | 0 |
| 2-OXOBUTYRIC ACID | 2-Oxobutyrate | 76 | 0.5524 | 0.75321 | 0.48712 | 0.6406 | | 0 |
| GLYCYL-GLYCINE | Glu-Glu | 52 | 0.0764 | 0.20434 | 0.05891 | 0.1588 | | 0 |
| LYSINE | Lys | 89 | 0.3653 | 0.62051 | 0.62264 | 0.7496 | | 0 |
| ASPARTIC ACID | Asp | 90 | 0.0295 | 0.1214 | 0.09959 | 0.233 | 7 | 1 |
| METHIONINE | Met | 75 | 0.3856 | 0.63739 | 0.3171 | 0.4737 | | 0 |
| P-HYDROXYPHENYLACETIC ACID | p-Hydroxyphenylacetate | 75 | 0.3063 | 0.54264 | 0.128 | 0.2784 | | 0 |
| AGMATINE | Agmatine | 66 | 0.8525 | 0.91124 | 0.87852 | 0.9232 | | 0 |
| 2-DEOXYRIBOSE 1-PHOSPHORIC ACID | 2-Deoxyribose 1-phosphate | 63 | 0.3958 | 0.63739 | 0.27727 | 0.428 | | 0 |
| PEPLOMYCIN | PEP | 85 | 0.1626 | 0.35379 | 0.20506 | 0.3634 | | 0 |
| DIHYDROXYACETONEPHOSPHORIC ACID | DHAP | 90 | 0.0052 | 0.05 | 0.01043 | 0.0494 | | 1 |
| GLYCOLIC ACID | Glycolate | 85 | 0.0336 | 0.13014 | 0.02913 | 0.0951 | | 1 |
| HISTAMINE | Histamine | 77 | 0.8398 | 0.91124 | 0.42786 | 0.6064 | | 0 |
| N-ACETYLLEUCINE | N-Acetylleucine | 28 | 0.4863 | 0.70123 | 0.91331 | 0.936 | | 0 |
| CYTIDINE DISODIUM 5′-MONOPHOSPHATE | CMP | 54 | 0.6165 | 0.8132 | 0.50663 | 0.6512 | | 0 |
| GUANINE | Guanine | 78 | 0.9532 | 0.98494 | 0.79472 | 0.8644 | | 0 |
| 4-METHYL-2-OXOPENTANOIC ACID | 4-Methyl-2-oxopentanoate | 90 | 0.0258 | 0.12032 | 0.04857 | 0.1401 | | 1 |
| N-ACETYLASPARTIC ACID | N-Acetylaspartate | 90 | 0.027 | 0.12032 | 0.08465 | 0.2135 | | 1 |
| TYROSINE | Tyr | 90 | 0.004 | 0.05 | 0.00997 | 0.0494 | 7 | 1 |
| SUCCINIC ACID | Succinate | 90 | 0.2212 | 0.43533 | 0.45454 | 0.619 | | 0 |
| GLYCEROPHOSPHORIC ACID | Glycerophosphate | 90 | 0.0179 | 0.09637 | 0.05316 | 0.1495 | | 1 |
| ALANYL-ALANINE | Ala-Ala | 62 | 0.5935 | 0.79179 | 0.62247 | 0.7496 | | 0 |
| 1,3-DIAMINOPROPANE | 1,3-Diaminopropane | 71 | 0.8458 | 0.91124 | 0.77566 | 0.8512 | 8 | 0 |
| 3-PHENYLPROPIONIC ACID | 3-Phenylpropionate | 80 | 0.7316 | 0.88078 | 0.71847 | 0.8242 | | 0 |
| CIS-ACONITATE | cis-Aconitate | 68 | 0.8461 | 0.91124 | 0.90341 | 0.9335 | | 0 |

TABLE 8-4

| | | RELATIVE CONCENTRATION (NO UNIT) | | | | | | | DETECTION |
|---|---|---|---|---|---|---|---|---|---|
| | | | | BREAST CANCER | | | | | RATIO (%) |
| | | | | ONLY BEFORE | | INCLUDING DURING | | | BREAST |
| | | HEALTHY SUBJECT | | TREATMENT | | TREATMENT | | | CANCER |
| | | | STANDARD | | STANDARD | | STANDARD | HEALTHY | BEFORE |
| NAME OF SUBSTANCE | | AVERAGE | DEVIATION | AVERAGE | DEVIATION | AVERAGE | DEVIATION | SUBJECT | TREATMENT |
| 3-HYDROXYBUTYRIC ACID | 3-Hydroxybutyrate | 0.647 | 0.52696 | 0.369 | 0.2321 | 0.4759 | 0.39905 | 20 | 37 |
| BENZOIC ACID | Benzoate | 1.332 | 0.9986 | 0.799 | 0.8965 | 0.8895 | 1.04938 | 18 | 27 |
| GUANOSINE | Guanosine | 0.024 | 0.03472 | 0.015 | 0.0197 | 0.0129 | 0.0182 | 10 | 19 |
| 6-PHOSPHOGLUCONIC ACID | 6-Phosphogluconate | 0.036 | 0.03115 | 0.031 | 0.0485 | 0.0288 | 0.03903 | 16 | 33 |
| URIDYLIC ACID | UMP | 0.012 | 0.01873 | 0.01 | 0.0136 | 0.0122 | 0.01936 | 8 | 18 |
| ADENOSINE | Adenosine | 0.019 | 0.02147 | 0.013 | 0.0212 | 0.0115 | 0.01764 | 11 | 22 |
| MUCIC ACID | Mucate | 0.032 | 0.04565 | 0.018 | 0.0317 | 0.0178 | 0.02731 | 11 | 18 |
| ETHANOLAMINEPHOSPHORIC ACID | Ethanolamine phosphate | 4.256 | 4.75927 | 3.189 | 3.9208 | 3.5534 | 4.83037 | 18 | 34 |
| ADIPATE | Adipate | 0.052 | 0.04601 | 0.03 | 0.0276 | 0.0385 | 0.04267 | 19 | 35 |
| 2-ISOPROPYLMALATE | 2-Isopropylmalate | 0.025 | 0.0254 | 0.014 | 0.013 | 0.0175 | 0.02347 | 20 | 36 |
| PHOSPHORYLCHLORINE | Phosphorylcholine | 0.547 | 0.35158 | 0.261 | 0.2648 | 0.3764 | 0.3664 | 20 | 31 |
| NICOTINATE | Nicotinate | 0.157 | 0.12343 | 0.098 | 0.1071 | 0.1272 | 0.13348 | 16 | 23 |
| 1-METHYL-2-PYRROLIDINONE | 1-Methyl-2-pyrrolidinone | 2.577 | 2.74352 | 1.798 | 2.1837 | 1.739 | 1.95496 | 20 | 36 |
| MALIC ACID | Malate | 0.181 | 0.11191 | 0.132 | 0.131 | 0.1531 | 0.13135 | 20 | 37 |
| PANTOTHENIC ACID | Pantothenate | 0.007 | 0.01177 | 0.004 | 0.006 | 0.007 | 0.01098 | 7 | 14 |
| N-ACETYLNEURAMINATE | N-Acetylneuraminate | 4.326 | 3.32649 | 2.13 | 1.8598 | 2.3366 | 1.94534 | 20 | 37 |
| HISTAMINE | His | 0.914 | 0.35757 | 0.542 | 0.309 | 0:609 | 0.33134 | 20 | 37 |
| FUMARIC ACID | Fumarate | 0.042 | 0.04031 | 0.03 | 0.0321 | 0.0327 | 0.03294 | 15 | 30 |
| 2-DEOXYGLUCOSE-6-PHOSPHORIC ACID | 2-Deoxyglucose 6-phosphate | 0.016 | 0.03298 | 0.01 | 0.0192 | 0.0093 | 0.01812 | 5 | 11 |
| CITRIC ACID | Citrate | 0.636 | 0.62144 | 0.449 | 0.672 | 0.5399 | 0.79022 | 20 | 35 |
| 4-ACETYLBUTYRIC ACID | 4-Acetylbutyrate | 0.013 | 0.02228 | 0.007 | 0.0149 | 0.0051 | 0.01133 | 6 | 11 |
| O-ACETYLCARNITINE | o-Acetylcarnitine | 0.092 | 0.0607 | 0.057 | 0.0544 | 0.0588 | 0.04852 | 20 | 37 |
| N-ACETYLGLUCOSAMINE-1-PHOSPHORIC ACID | N-Acetylglucosamine 1-phosphate | 0.021 | 0.01561 | 0.01 | 0.0111 | 0.0103 | 0.01125 | 17 | 26 |
| SEDOHEPTULOSE-7-PHOSPHORIC ACID | S7P | 0.038 | 0.0291 | 0.024 | 0.0242 | 0.0263 | 0.02339 | 16 | 29 |
| HEPTANOIC ACID | Heptanoate | 0.032 | 0.03981 | 0.015 | 0.0218 | 0.0165 | 0.02305 | 15 | 25 |
| CREATININE | Creatinine | 0.364 | 0.23931 | 0.221 | 0.2029 | 0.2462 | 0.22261 | 20 | 37 |
| 2,5-DIHYDROXYBENZONATE | 2,5-Dihydroxybenzoate | 0.04 | 0.05452 | 0.021 | 0.0352 | 0.0334 | 0.07638 | 9 | 17 |
| CYTIDINE | Cytidine | 0.037 | 0.4016 | 0.022 | 0.0278 | 0.0192 | 0.02594 | 14 | 22 |
| ARGININE | Arg | 1.147 | 0.61391 | 0.775 | 0.7669 | 0.8378 | 0.72021 | 20 | 37 |
| SYRINGIC ACID | Syringate | 0.13 | 0.08544 | 0.085 | 0.0942 | 0.0843 | 0.08747 | 20 | 34 |

| | | DETECTION | MANN-WHITNEY TEST | | | | | |
|---|---|---|---|---|---|---|---|---|
| | | RATIO (%) | ONLY BEFORE | | INCLUDING | | | |
| | | BREAST | TREATMENT | | DURING | | | |
| | | CANCER | vs HEALTHY | | TREATMENT vs | | | |
| | | INCLUDING DURING | SUBJECT | | HEALTHY SUBJECT | | PUBLICLY | |
| NAME OF SUBSTANCE | | TREATMENT | P VALUE | Q VALUE | P VALUE | Q VALUE | KNOWN | SIGNIFICANT |
| 3-HYDROXYBUTYRIC ACID | 3-Hydroxybutyrate | 90 | 0.037 | 0.1319 | 0.14 | 0.294 | | 1 |
| BENZOIC ACID | Benzoate | 67 | 0.027 | 0.1203 | 0.024 | 0.085 | | 1 |
| GUANOSINE | Guanosine | 42 | 0.624 | 0.8149 | 0.345 | 0.505 | | 0 |
| 6-PHOSPHOGLUCONIC ACID | 6-Phosphogluconate | 77 | 0.258 | 0.4929 | 0.227 | 0.386 | | 0 |
| URIDYLIC ACID | UMP | 42 | 0.848 | 0.9112 | 0.744 | 0.824 | | 0 |
| ADENOSINE | Adenosine | 49 | 0.455 | 0.6824 | 0.272 | 0.427 | | 0 |
| MUCIC ACID | Mucate | 49 | 0.293 | 0.535 | 0.358 | 0.516 | | 0 |
| ETHANOLAMINEPHOSPHORIC ACID | Ethanolamine phosphate | 84 | 0.219 | 0.4353 | 0.28 | 0.428 | | 0 |
| ADIPATE | Adipate | 87 | 0.03 | 0.1214 | 0.093 | 0.221 | | 1 |
| 2-ISOPROPYLMALATE | 2-Isopropylmalate | 87 | 0.029 | 0.1214 | 0.02 | 0.074 | | 1 |
| PHOSPHORYLCHLORINE | Phosphorylcholine | 81 | 6E−04 | 0.0137 | 0.013 | 0.054 | | 1 |
| NICOTINATE | Nicotinate | 63 | 0.068 | 0.1926 | 0.211 | 0.369 | | 0 |
| 1-METHYL-2-PYRROLIDINONE | 1-Methyl-2-pyrrolidinone | 87 | 0.107 | 0.2553 | 0.092 | 0.221 | | 0 |
| MALIC ACID | Malate | 90 | 0.057 | 0.1702 | 0.189 | 0.345 | | 0 |
| PANTOTHENIC ACID | Pantothenate | 42 | 0.787 | 0.9032 | 0.549 | 0.694 | | 0 |
| N-ACETYLNEURAMINATE | N-Acetylneuraminate | 90 | 0.005 | 0.05 | 0.006 | 0.043 | | 1 |
| HISTAMINE | His | 90 | 2E−04 | 0.0099 | 9E−04 | 0.022 | | 1 |
| FUMARIC ACID | Fumarate | 72 | 0.392 | 0.6374 | 0.491 | 0.641 | | 0 |
| 2-DEOXYGLUCOSE-6-PHOSPHORIC ACID | 2-Deoxyglucose 6-phosphate | 24 | 0.975 | 0.9907 | 0.869 | 0.921 | | 0 |
| CITRIC ACID | Citrate | 86 | 0.075 | 0.2043 | 0.138 | 0.294 | | 0 |
| 4-ACETYLBUTYRIC ACID | 4-Acetylbutyrate | 25 | 0.687 | 0.8518 | 0.449 | 0.619 | | 0 |
| O-ACETYLCARNITINE | o-Acetylcarnitine | 89 | 0.01 | 0.0719 | 0.009 | 0.049 | | 1 |
| N-ACETYLGLUCOSAMINE-1-PHOSPHORIC ACID | N-Acetylglucosamine 1-phosphate | 63 | 0.006 | 0.0508 | 0.002 | 0.025 | | 1 |
| SEDOHEPTULOSE-7-PHOSPHORIC ACID | S7P | 72 | 0.068 | 0.1926 | 0.112 | 0.252 | | 0 |
| HEPTANOIC ACID | Heptanoate | 56 | 0.117 | 0.2723 | 0.086 | 0.213 | | 0 |
| CREATININE | Creatinine | 90 | 0.005 | 0.05 | 0.01 | 0.049 | | 1 |
| 2,5-DIHYDROXYBENZONATE | 2,5-Dihydroxybenzoate | 38 | 0.438 | 0.6704 | 0.483 | 0.641 | | 0 |
| CYTIDINE | Cytidine | 49 | 0.178 | 0.373 | 0.054 | 0.149 | | 0 |
| ARGININE | Arg | 90 | 0.013 | 0.083 | 0.027 | 0.092 | | 1 |
| SYRINGIC ACID | Syringate | 82 | 0.039 | 0.1319 | 0.015 | 0.062 | | 1 |

Even when the whole concentration of saliva of elderly patient is increased, using glutamine that is a substance that correlates with the most metabolites and can be detected in all of the samples as a concentration-correcting marker makes it possible to distinguish a subject with cancer from a healthy subject by eliminating the influence of concentration variations by this method. On the other hand, people equal to or older than 70 years of age have a trend of increasing the whole concentration of saliva. Therefore, when the absolute concentration is used, the construction of a model using only data of people less than 70 years of age leads to a highly accurate separation.

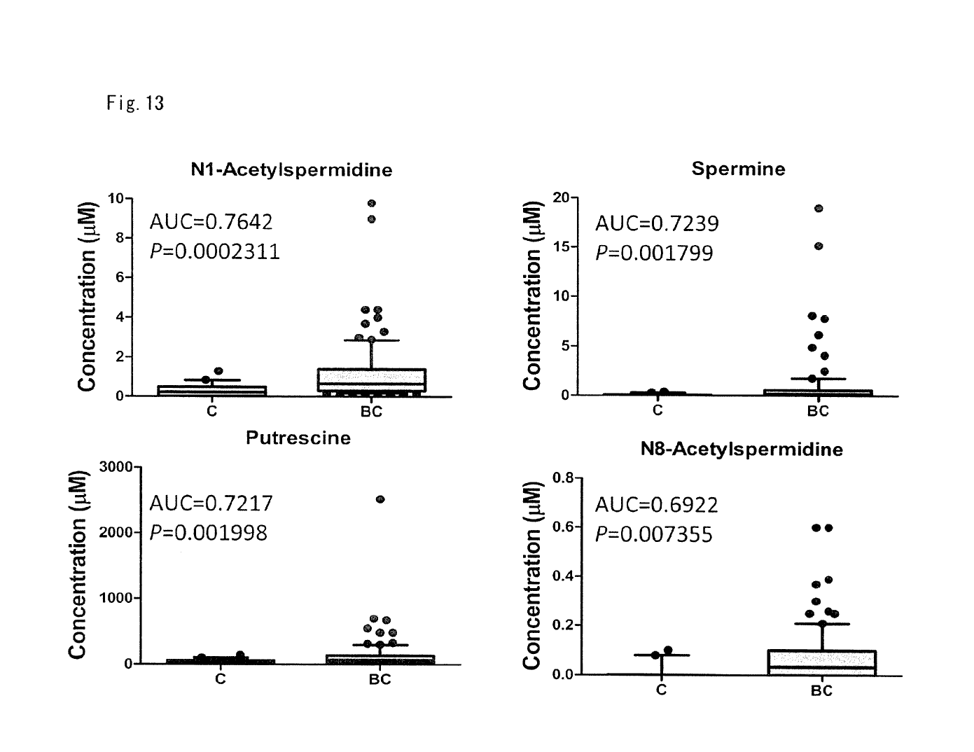

A substance belonging to polyamines among substances that give a significant difference between the healthy subjects (C, n=20) and the patients with breast cancer (BC, all the cases including before treatment, n=90) is shown in FIG. 13.

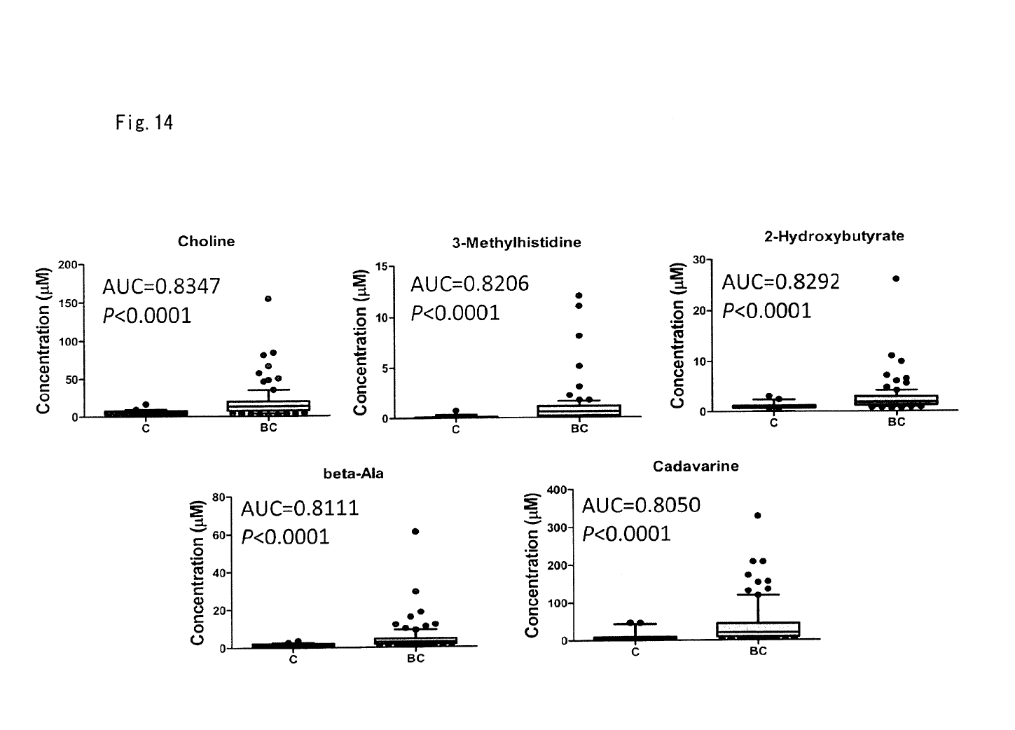

Examples (the top five substances with a smaller P value) of substances other than polyamines among the substances that give a significant difference between the healthy subjects (C, n=20) and the patients with breast cancer (BC, all the cases including before treatment, n=90) are shown in FIG. 14.

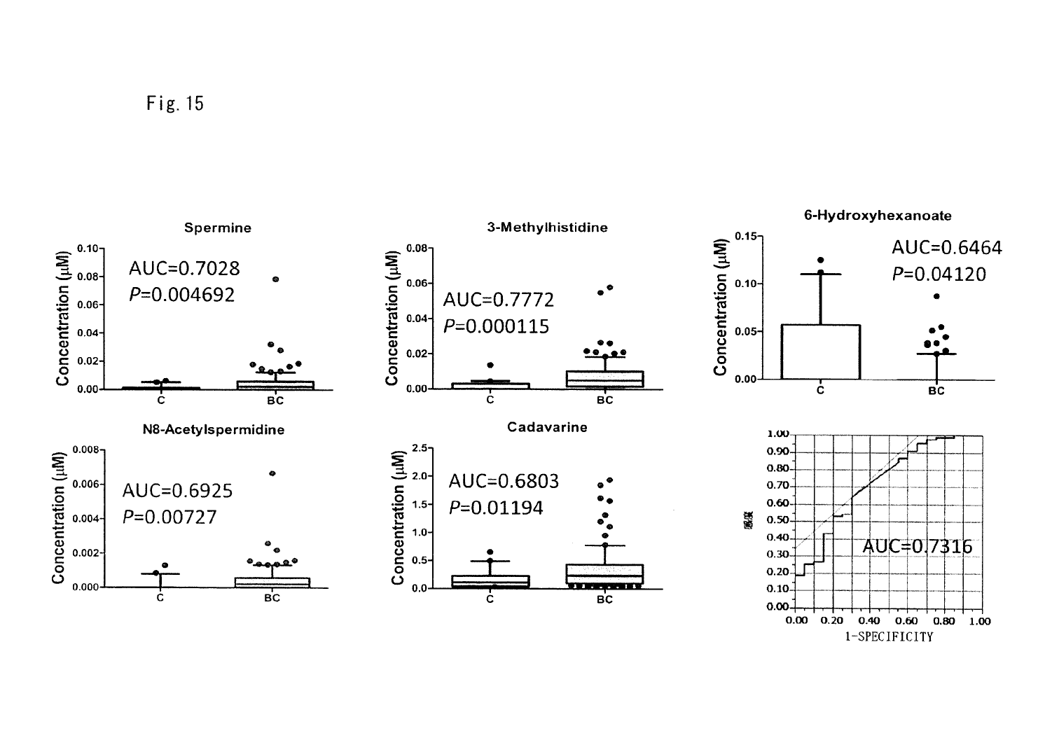

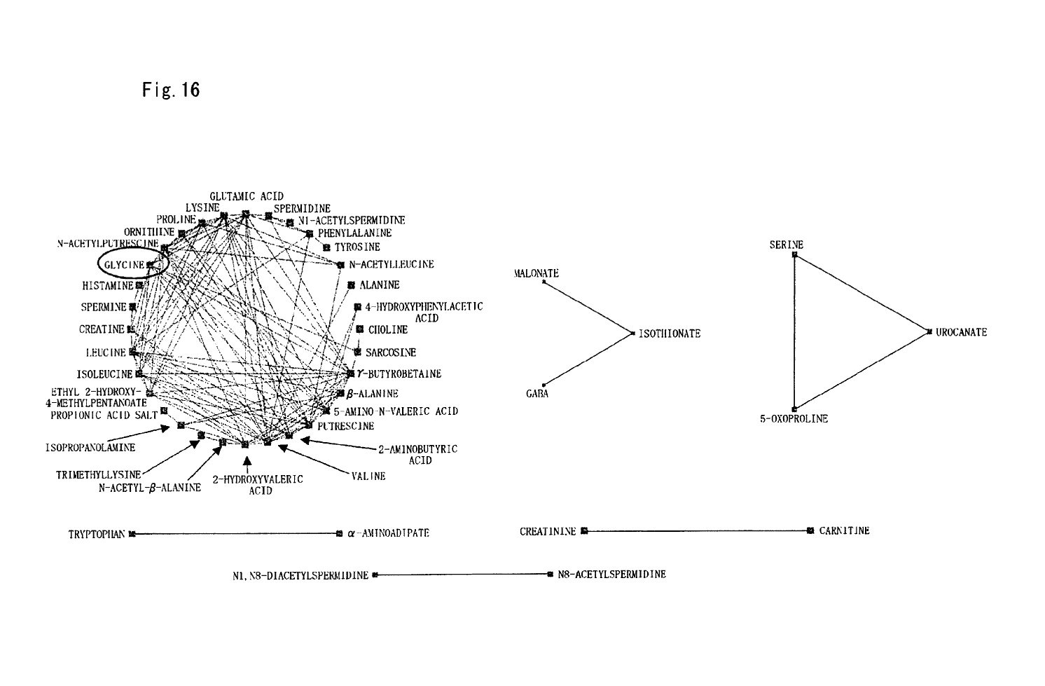

Substances that give a significant difference (p<0.05) between the healthy subjects (C, 20 cases) and the patients with breast cancer (BC, 90 cases) regardless of the presence or absence of concentration correction are shown in FIG. 15. A network diagram (shown in FIG. 16) was formed using all the cases of all the samples (20 cases of C and 90 cases of BC). A concentration correction substance, or Gly (glycine, expressed in o in the drawing) was determined. When concentration correction with this concentration correction marker was not performed, 73 substances exhibited a significant difference. When concentration correction was performed (the concentration of metabolite of interest was divided by the concentration of Gly), 35 substances exhibited a significant difference. Among the substances, 11 substances exhibited a significant difference regardless of the presence or absence of concentration correction. The top five substances that had a smaller P value are shown. An ROC curve at which an MLR model was formed using two substances of spermine and 6-hydaroxyhexanoate is shown in the lower right.

Next, a biomarker for oral cancer will be described.

Substances in which the absolute concentrations of metabolites in saliva exhibit a difference between subjects with oral cancer and healthy subjects are shown in Tables 9-1 and 9-2. The healthy subjects were 20 cases, and patients with breast cancer were 20 cases. For the healthy subjects, saliva was collected 1.5 hours after eating, and for the patients with cancer, saliva was collected two times, before eating (in a fasting state from the previous night) and 1.5 hours after eating. By comparing either of them, a P value was calculated using the Mann-Whitney test, and a Q value was calculated using the false discovery rate (FDR). Substances of Q<0.05 were listed.

TABLE 9-1

| | | | | | | | | TEST (COMPARISON | | | | |
|---|---|---|---|---|---|---|---|---|---|---|---|---|
| | | HEALTHY | | | | | | WITH HEALTHY SUBJECTS) | | | | |
| | | SUBJECT | | ORAL CANCER | | | | CANCER | | CANCER | | |
| | | 1.5 HOURS | | 1.5 HOURS | | ON EMPTY | | (1.5 HOURS/ | | (ON EMPTY/ | | |
| | | AFTER DIET | | AFTER DIET | | STOMACH | | AFTER DIET) | | STOMACH) | | PUBLICLY |
| COMPOUND | NAME OF SUBSTANCE | AVERAGE | S.D. | AVERAGE | S.D. | AVERAGE | S.D. | P-value | Q-value | P-value | Q-value | KNOWN |
| Gly-Gly | GLYCYL-GLYCINE | 0.4069 | 0.9342 | 1.058 | 1.70904 | 2.6807 | 3.3424 | 0.01231 | 0.08136 | 7.3E−05 | 0.0028 | |
| Choline | CHOLINE | 9.8191 | 13.615 | 16.57 | 10.5053 | 23.982 | 18.487 | 0.00196 | 0.03719 | 9.5E−05 | 0.0029 | 9 |
| Citrulline | CITRULLINE | 15.509 | 17.115 | 32.87 | 33.9328 | 58.026 | 59.93 | 0.04298 | 0.16753 | 0.00013 | 0.0032 | |
| gamma-Butyrobetaine | γ-BUTYROBETAINE | 3.0834 | 3.8972 | 5.898 | 5.71459 | 8.116 | 5.7123 | 0.01809 | 0.10186 | 0.00015 | 0.0032 | |
| 3-Phenyllactate | 3-PHENYLLACTATE | 1.7552 | 3.8247 | 2.227 | 2.43789 | 4.436 | 5.4298 | 0.15394 | 0.2945 | 0.00024 | 0.004 | |
| Butanoate | BUTYRIC ACID | 63.135 | 53.789 | 368.9 | 572.732 | 276.63 | 290.83 | 0.00195 | 0.03719 | 0.00027 | 0.004 | |
| Hexanoate | HEXANOIC ACID | 13.695 | 13.216 | 38.52 | 65.1854 | 71.359 | 106.92 | 0.34078 | 0.45043 | 0.0003 | 0.004 | |
| Met | METHIONINE | 1.372 | 3.3692 | 2.48 | 4.46552 | 7.1068 | 10.922 | 0.12541 | 0.28034 | 0.00032 | 0.004 | |
| Hypoxanthine | HYPOXANTHINE | 5.4485 | 8.9829 | 12.46 | 16.7868 | 18.403 | 21.808 | 0.0239 | 0.11717 | 0.00034 | 0.004 | |
| Spermidine | SPERMIDINE | 1.813 | 1.7732 | 3.705 | 5.16895 | 4.6425 | 2.7304 | 0.31408 | 0.42626 | 0.00043 | 0.0042 | |
| Val | VALINE | 13.282 | 20.557 | 27.63 | 47.2029 | 47.09 | 63.858 | 0.1207 | 0.27586 | 0.00044 | 0.0042 | 7 |
| Glu | GLUTAMIC ACID | 43.09 | 90.355 | 54.12 | 65.7928 | 93.418 | 88.182 | 0.31408 | 0.42626 | 0.00044 | 0.0042 | 7 |
| Trp | TRYPTOPHAN | 1.6971 | 2.9918 | 2.367 | 2.83711 | 5.3687 | 6.4 | 0.10745 | 0.26082 | 0.00048 | 0.0042 | |
| Ala | ALANINE | 40.573 | 68.685 | 73.86 | 78.6906 | 137.25 | 145.91 | 0.00672 | 0.06109 | 0.0005 | 0.0042 | 7 |
| Asp | ASPARTIC ACID | 19 | 30.334 | 27.79 | 28.8413 | 45.057 | 41.162 | 0.1207 | 0.27586 | 0.00104 | 0.0083 | |
| Piperidine | PIPERIDINE | 0.1498 | 0.2574 | 1.321 | 2.36202 | 1.3152 | 2.5325 | 0.00501 | 0.06109 | 0.00121 | 0.0083 | 7 |
| Isopropanolamine | ISOPROPANOLAMINE | 0.6591 | 0.5352 | 1.688 | 1.72126 | 1.5766 | 0.9826 | 0.06715 | 0.20118 | 0.00121 | 0.0083 | |
| Ala-Ala | ALANYL-ALANINE | 0.8831 | 1.1716 | 1.732 | 2.46196 | 2.8654 | 3.1707 | 0.37911 | 0.48424 | 0.00123 | 0.0083 | |
| N,N-Dimethylglycine | N,N-DIMETHYLGLYCINE | 0.2266 | 0.5635 | 0.43 | 0.47283 | 0.5607 | 0.3983 | 0.00596 | 0.06109 | 0.00126 | 0.0083 | |
| N1-Acetylspermidine | N1-ACETYLSPERMIDINE | 0.7048 | 0.9459 | 1.337 | 1.27463 | 2.0556 | 2.4166 | 0.01327 | 0.08387 | 0.00143 | 0.0087 | |
| N1,N8-Diacetylspermidine | N1-,N8-DIACETYLSPERMIDINE | 0.5098 | 1.6679 | 0.336 | 0.33971 | 0.5473 | 0.5625 | 0.03538 | 0.14152 | 0.00145 | 0.0087 | |
| N8-Acetylspermidine | N8-ACETYLSPERMIDINE | 0.0524 | 0.0882 | 0.111 | 0.1197 | 0.1356 | 0.1038 | 0.06919 | 0.20192 | 0.00148 | 0.0087 | |
| 2AB | α-AMINOBUTYRIC ACID | 1.6132 | 1.5683 | 3.175 | 4.32823 | 6.9715 | 13.599 | 0.14171 | 0.2945 | 0.00166 | 0.0088 | |
| Trimethylamine N-oxide | TRIMETHYLAMINE-N-OXIDE | 0.1483 | 0.2186 | 0.596 | 0.6416 | 0.8705 | 1.2581 | 0.00337 | 0.05691 | 0.00175 | 0.0088 | |
| N-Acetylaspartate | N-ACETYLASPARTIC ACID | 0.9277 | 0.817 | 2.256 | 2.45157 | 2.2515 | 1.7815 | 0.01435 | 0.08387 | 0.00185 | 0.0088 | |
| Adenine | ADENINE | 0.6068 | 0.6117 | 0.845 | 0.73556 | 1.2708 | 0.8954 | 0.06377 | 0.20118 | 0.00185 | 0.0088 | |
| Thr | THREONINE | 7.3662 | 12.091 | 12.76 | 14.0435 | 19.827 | 18.913 | 0.02831 | 0.12656 | 0.00185 | 0.0088 | 7 |
| 2-Hydroxypentanoate | 2-HYDROXIVALERIC ACID | 10.065 | 22.066 | 12.13 | 13.4737 | 16.866 | 20.227 | 0.08023 | 0.21396 | 0.00193 | 0.0089 | |
| Putrescine(1,4-Butanediamine) | PUTRESCINE | 55.793 | 65.975 | 139.1 | 212.235 | 140.18 | 122.54 | 0.04909 | 0.18199 | 0.00207 | 0.009 | |
| Ile | ISOLEUCINE | 5.4605 | 9.7987 | 10.74 | 19.3536 | 17.599 | 23.355 | 0.0675 | 0.20118 | 0.00207 | 0.009 | 7 |
| 3PG | 3-PHOSPHOGLYCERIC ACID BARIUM SALT | 2.9479 | 4.4668 | 5.387 | 4.81451 | 5.5895 | 3.7831 | 0.00733 | 0.06109 | 0.00231 | 0.0092 | |
| Gln | GLUTAMINE | 21.627 | 22.327 | 38.27 | 36.3946 | 66.62 | 65.148 | 0.07178 | 0.20192 | 0.00231 | 0.0092 | 7 |
| beta-Ala | β-ALANINE | 2.2944 | 2.0961 | 3.48 | 3.29484 | 4.9516 | 3.9527 | 0.10216 | 0.25881 | 0.00231 | 0.0092 | 7 |
| 3-Phenylpropionate | 3-PHENYLPROPIONIC ACID | 8.6553 | 9.8714 | 29.14 | 48.6612 | 53.281 | 74.39 | 0.22064 | 0.36454 | 0.00256 | 0.0094 | |
| Ser | SERINE | 16.853 | 20.939 | 34.11 | 35.0987 | 45.686 | 40.867 | 0.0143 | 0.08387 | 0.00257 | 0.0094 | |

TABLE 9-2

| | | | | | | | | TEST (COMPARISON | | | | |
|---|---|---|---|---|---|---|---|---|---|---|---|---|
| | | HEALTHY | | | | | | WITH HEALTHY SUBJECTS) | | | | |
| | | SUBJECT | | ORAL CANCER | | | | CANCER | | CANCER | | |
| | | 1.5 HOURS | | 1.5 HOURS | | ON EMPTY | | (1.5 HOURS/ | | (ON EMPTY/ | | |
| | | AFTER DIET | | AFTER DIET | | STOMACH | | AFTER DIET) | | STOMACH) | | PUBLICLY |
| COMPOUND | NAME OF SUBSTANCE | AVERAGE | S.D. | AVERAGE | S.D. | AVERAGE | S.D. | P-value | Q-value | P-value | Q-value | KNOWN |
| 1-Methylnicotinamide | 1-METHYLNICOTINAMIDE | 0 | 0 | 0.0863 | 0.1482 | 0.0447 | 0.061 | 0.00093 | 0.0283 | 0.002658 | 0.0093969 | |
| 3-Hydroxy-3-methylglutarate | 3-HYDROXY-3-METHYLGLUTARIC ACID | 0 | 0 | 0.1326 | 0.2051 | 0.1545 | 0.263 | 0.00093 | 0.0283 | 0.002658 | 0.0093969 | |
| Guanine | GUANINE | 0.8532 | 0.8635 | 2.0106 | 2.3806 | 2.1938 | 2.504 | 0.08023 | 0.214 | 0.002701 | 0.0093969 | |
| 3-(4-Hydroxyphenyl)propionate | 3-(4-HYDROXYPHENYL)PROPIONIC ACID | 5.7327 | 5.3987 | 16.824 | 25.171 | 26.897 | 26.52 | 0.22686 | 0.3665 | 0.002948 | 0.0099587 | |
| 4-Methylbenzoate | 4-METHYLBENZOATE | 13.693 | 16.348 | 29.523 | 44.709 | 40.875 | 35.76 | 0.15324 | 0.2945 | 0.003242 | 0.0107135 | |
| Ru5P | RIBULOSE-5-PHOSPHORIC ACID | 3.6439 | 3.1654 | 6.2273 | 3.6308 | 6.4447 | 3.509 | 0.00427 | 0.0611 | 0.003529 | 0.0111736 | |
| Cadaverine | CADAVERINE | 17.118 | 26.089 | 64.655 | 111.69 | 70.095 | 93.24 | 0.03264 | 0.1341 | 0.003529 | 0.0111736 | 7 |
| alpha-Aminoadipate | α-AMINOADIPIC ACID | 2.2491 | 4.0828 | 2.9071 | 1.7281 | 3.6911 | 4.177 | 0.00511 | 0.0611 | 0.00362 | 0.011228 | |
| N-epsilon-Acetyllysine | N6-ACETYLLYSINE | 0.2091 | 0.532 | 0.3292 | 0.4221 | 0.6021 | 0.626 | 0.07152 | 0.2019 | 0.004421 | 0.0131769 | |
| Glucosamine | GLUCOSAMINE | 0.0982 | 0.2061 | 0.4062 | 0.7291 | 0.7409 | 1.101 | 0.15617 | 0.2945 | 0.004612 | 0.0133543 | |
| Pipecolate | PICOLINIC ACID | 0.5181 | 0.5391 | 0.7482 | 0.7248 | 1.5685 | 1.624 | 0.27202 | 0.4195 | 0.004658 | 0.0133543 | 79 |
| Cystine | CYSTINE | 0.1372 | 0.4283 | 0.2625 | 0.288 | 0.4851 | 0.588 | 0.01123 | 0.0813 | 0.005031 | 0.0141617 | |
| Leu | LEUCINE | 15.883 | 26.949 | 27.312 | 49.448 | 47.753 | 73.1 | 0.14171 | 0.2945 | 0.005288 | 0.0146144 | 7 |
| Carnosine | CARNOSINE | 0.2446 | 0.406 | 0.092 | 0.1094 | 0.0635 | 0.201 | 0.30422 | 0.4242 | 0.005549 | 0.0150626 | |
| Urocanate | UROCANIC ACID | 3.3151 | 4.3875 | 5.4696 | 8.6626 | 6.7838 | 7.392 | 0.14171 | 0.2945 | 0.005833 | 0.0152872 | |
| Phe | PHENYLALANINIE | 22.976 | 38.395 | 24.975 | 26.739 | 43.982 | 42.62 | 0.34078 | 0.4504 | 0.005833 | 0.0152872 | |
| 2-Deoxyribose 1-phosphate | 2-DEOXYRIBOSE-1-PHOSPHORIC ACID | 0 | 0 | 0.4503 | 1.456 | 1.3412 | 3.671 | 0.0198 | 0.1075 | 0.005141 | 0.015558 | |
| CMP | CYTIDINE DISODIUM 5′-MONOPHOSPHATE | 0 | 0 | 0.0262 | 0.0806 | 0.6152 | 1.603 | 0.16259 | 0.3014 | 0.006141 | 0.015558 | |
| p-Hydroxyphenylacetate | p-HYDROXYPHENYLACETIC ACID | 9.3775 | 15.199 | 23.122 | 26.812 | 32.256 | 31.62 | 0.02914 | 0.1266 | 0.006704 | 0.0167057 | |
| 3-Hydroxybutyrate | POLYHYDROXYBUTYRIC ACID | 5.849 | 7.9585 | 9.1822 | 7.9574 | 9.7319 | 5.422 | 0.06574 | 0.2012 | 0.008441 | 0.0205966 | |
| N-Acetylputrescine | N-ACETYLPUTRESCINE | 3.7027 | 4.2148 | 6.7508 | 9.5333 | 8.5994 | 8.382 | 0.14928 | 0.2945 | 0.008537 | 0.0205966 | |
| 7-Methylguanine | 7-METHYLGUANINE | 0.0973 | 0.169 | 0.1594 | 0.1784 | 0.2467 | 0.17 | 0.15081 | 0.2945 | 0.008981 | 0.021304 | |
| Inosine | INOSINE | 1.1818 | 5.2853 | 1.1392 | 2.1327 | 0.932 | 1.333 | 0.00764 | 0.0611 | 0.00911 | 0.021304 | |
| Lys | LYSINE | 54.872 | 77.058 | 59.839 | 61.894 | 107.38 | 92.02 | 0.35465 | 0.4647 | 0.010257 | 0.0232693 | |
| DHAP | DIHYDROXYACETONEPHOSPHORIC ACID | 9.8189 | 12.418 | 14.832 | 12.189 | 15.183 | 10.62 | 0.02633 | 0.1213 | 0.011224 | 0.0250892 | |
| 3-Methylhistidine | 3-METHYLHISTIDINE | 0.269 | 0.3248 | 0.3945 | 0.198 | 0.6527 | 0.531 | 0.10982 | 0.2608 | 0.011398 | 0.0251085 | |
| Carbamoylaspartate | CARBAMOYLASPARTIC ACID | 0.1314 | 0.3286 | 0.3321 | 0.6735 | 0.5884 | 0.684 | 0.27515 | 0.4195 | 0.012219 | 0.0259014 | |
| Creatinine | CREATINE | 4.5834 | 2.8466 | 5.6183 | 2.1009 | 6.9995 | 4.008 | 0.05589 | 0.1976 | 0.012269 | 0.0259014 | |
| 1-Methyl-2-pyrrolidinone | N-METHYL-2-PYRROLIDONE | 0 | 0 | 3.393 | 4.145 | 1.6277 | 2.739 | 0.00093 | 0.0283 | 0.013779 | 0.0286895 | |
| Pyruvate | PYRUVIC ACID | 71.74 | 129.01 | 95.867 | 72.494 | 100.95 | 72.28 | 0.00733 | 0.0611 | 0.014053 | 0.0288663 | |
| Carnitine | CARNITINE | 1.3784 | 1.4646 | 1.6847 | 1.0058 | 2.2939 | 1.956 | 0.0524 | 0.1896 | 0.014613 | 0.0292252 | 79 |
| Propionate | PROPIONIC ACID | 212.01 | 162.5 | 503.43 | 443.17 | 444.31 | 328.5 | 0.00733 | 0.0611 | 0.01733 | 0.0333437 | |
| 5-Aminovalerate | 5-AMINOVALERIC ACID | 353.84 | 383.45 | 680.09 | 897.61 | 681.35 | 530.6 | 0.0675 | 0.2012 | 0.01733 | 0.0333437 | |
| N-Acetylornithine | N-ACETYLORNITHINE | 0.15 | 0.3066 | 0.3977 | 0.6845 | 0.5015 | 0.628 | 0.15694 | 0.2945 | 0.020103 | 0.0377233 | |
| Tyr | TYROSINE | 29.353 | 30.264 | 39.195 | 38.122 | 49.956 | 32.98 | 0.30125 | 0.4242 | 0.020467 | 0.0379391 | 7 |
| 5-Oxoproline | 5-OXOPROLINE | 11.522 | 26.849 | 11.424 | 13.484 | 14.236 | 20.71 | 0.06343 | 0.2012 | 0.022209 | 0.0406711 | |
| Creatinine | CREATININE | 30.546 | 53.384 | 26.173 | 13.642 | 33.368 | 35.71 | 0.04595 | 0.1746 | 0.024074 | 0.0434287 | |
| Homoserine | HOMOSERINE | 0.3548 | 0.5828 | 0.657 | 0.7476 | 0.6454 | 0.537 | 0.07306 | 0.2019 | 0.024286 | 0.0434287 | |
| Fumarate | FUMARIC ACID | 0.4579 | 1.4637 | 1.5633 | 2.3957 | 0.703 | 0.883 | 0.01212 | 0.0814 | 0.025797 | 0.0455463 | |
| Gly | GLYCINE | 130.77 | 167.9 | 157.76 | 117.97 | 222.74 | 193.5 | 0.14928 | 0.2945 | 0.026069 | 0.0455463 | |

A substance in which “7” or “9” is described in the column labeled “Publicly Known” is a known substance disclosed in Non-Patent Literature 7 or 9.

Herein, the patients with oral cancer included stages I to IVa, and include oral squamous cell carcinoma (17 cases), malignant melanoma (2 cases), and adenoid cystic carcinoma (1 case). Spermine, spermidine, or acetylated spermine or spermidine consistently have a high concentration in comparison of an oral cancer tissue sample obtained during surgery and a healthy part in a vicinity of the oral cancer tissue.

For example, choline (second substance from the top in Table) among the substances is a known substance in Non-Patent Literatures 7 and 9, and an increase in the concentration of the substance in saliva has been confirmed. However, oral cancer can be identified with high accuracy by a mathematical model combined with a plurality of novel markers by the same procedure as those in pancreatic cancer and breast cancer. The substance is increased in oral cancer, but is not increased in breast cancer. Therefore, when the substance is included as a variable of the mathematical model, the specific type of cancer can be expressed.

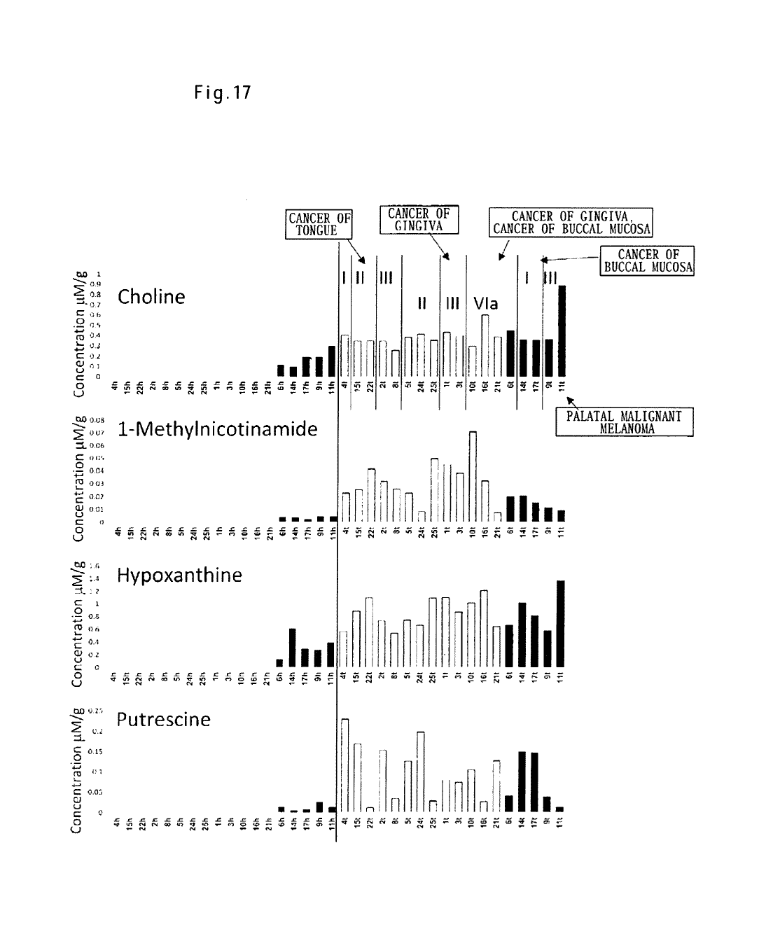

The concentrations of metabolites in the cancer tissue sample obtained during surgery of oral cancer and the healthy tissue sample near the cancer tissue sample (herein, the concentration corrected with the weight of the tissue in μM/g is used) are shown in FIG. 17. In the drawing, the healthy tissue is at a left part and the cancer tissue is at a right part. An extent of progression (grade) of cancer is represented by I, II, III, and Via. The drawing shows some substances that have a significant difference between the healthy part and the cancer part.

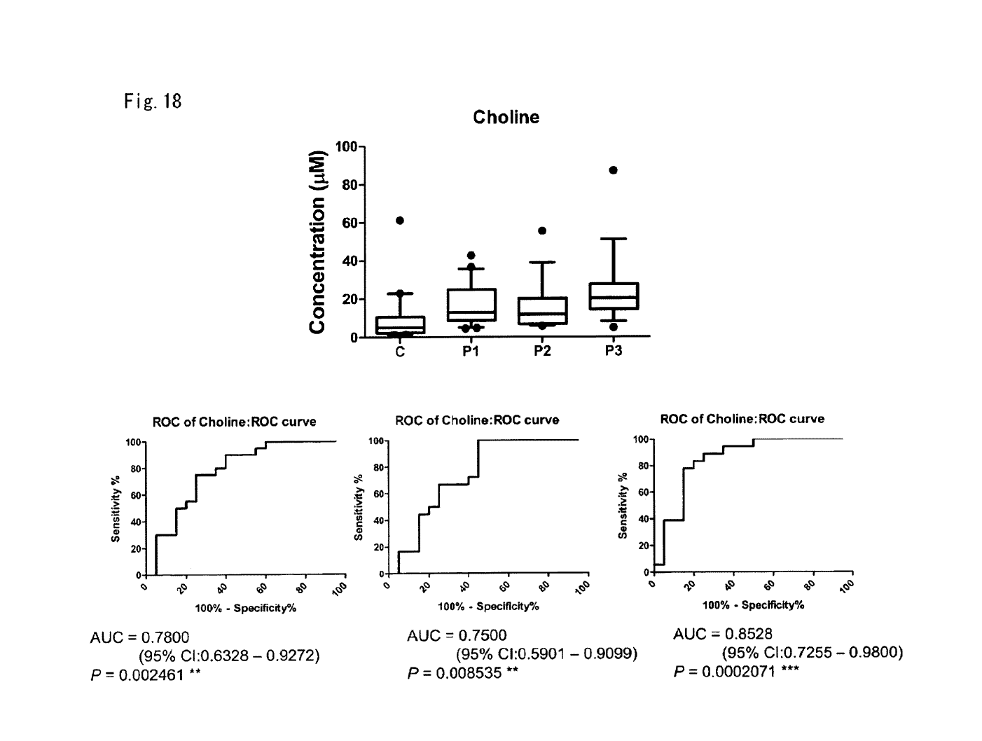

A difference in the concentration of saliva between the patients with oral cancer and the healthy subjects (C) when a method of collecting saliva in the patients with oral cancer was changed is shown in FIG. 18. For the healthy subjects, choline (Choline) that had the smallest P value in comparison of saliva from the patients with oral cancer is expressed as an example. For the healthy subjects (C), saliva was collected 1.5 hours after eating. For oral cancer, saliva was collected from the same patients, and saliva was collected 1.5 hours after eating as P1, collected 3.5 hours after eating as P2, and collected during fasting (before breakfast) as P3.

Table 10 shows results in which the absolute concentrations of polyamines and hypoxanthine were measured using saliva collected from 17 healthy subjects, 21 patients with pancreatic cancer, 16 patents with breast cancer, and 20 patients with oral cancer during fasting (hungry from 9:00 of previous night, no eating on the collection day) by liquid chromatography-mass spectrometer (LC-MS). A P value for evaluation of difference in average was calculated using the Student's t-test as a parametric test because the number of cases was small.

Results of determination of polyamines and hypoxanthine (Hypoxanthine) as a metabolite other than the polyamines are shown in Table 10. Among the polyamines, when N1,N12-diacetylspermine (N1,N12-diacetylspermine) was measured using CE-TOFMS, the peak thereof overlapped the peak of another substance. When LC-qTOFMS was used, the peak of N1,N12-diacetylspermine and the peak of the other substance could be separately measured. Herein, only the samples that were collected during fasting were used. The quantitative values determined for 17 cases of healthy subjects, 21 cases of pancreatic cancer, 18 cases of oral cancer, and 16 cases of breast cancer are described (the unit of quantitative value is μM).

TABLE 10

| | | HEALTHY | | PANCREATIC | | | | | | | | |
|---|---|---|---|---|---|---|---|---|---|---|---|---|
| | | SUBJECT | | CANCER | | | ORAL CANCER | | | BREAST CANCER | | |
| | | AVER- | | AVER- | | | AVER- | | | AVER- | | |
| COMPOUND | JAPANESE NAME | AGE | SD | AGE | SD | P-value | AGE | SD | P-value | AGE | SD | P-value |
| Hypoxanthine | HYPOXANTHINE | 1.088 | 1.421 | 3.245 | 3.158 | 0.00914 | 6.569 | 3.410 | 0.01718 | 1.496 | 1.643 | 0.452478 |
| Spermidine | SPERMIDINE | 1.571 | 1.553 | 3.943 | 2.957 | 0.003346 | 4.756 | 3.889 | 0.00269 | 5.119 | 6.958 | 0.003078 |
| N8-Acetylspermidine | N8-ACETYL- | 0.017 | 0.030 | 0.047 | 0.056 | 0.046547 | 0.088 | 0.114 | 0.01942 | 0.065 | 0.146 | 0.211951 |
| | SPERMIDINE | | | | | | | | | | | |
| N1-Acetylspermidine | N1-ACETYL- | 0.039 | 0.052 | 0.132 | 0.124 | 0.004252 | 0.482 | 0.689 | 0.01453 | 0.224 | 0.372 | 0.06753 |
| | SPERMIDINE | | | | | | | | | | | |
| Spermine | SPERMINE | 0.147 | 0.176 | 1.328 | 1.937 | 0.011404 | 2.526 | 3.351 | 0.00789 | 0.636 | 0.539 | 0.013041 |
| N1,N8- | N1-,N8-DIACETYL- | 0.090 | 0.118 | 0.184 | 0.153 | 0.038241 | 0.223 | 0.336 | 0.12931 | 0.219 | 0.292 | 0.11577 |
| Diacetylspermidine | SPERMIDINE | | | | | | | | | | | |
| N1-Acetylspermine | N1-ACETYL- | 0.024 | 0.033 | 0.114 | 0.101 | 0.000769 | 0.242 | 0.379 | 0.02649 | 0.099 | 0.133 | 0.042624 |
| | SPERMINE | | | | | | | | | | | |
| N1,N12- | N1,N12-DIACETYL- | 0.068 | 0.103 | 0.189 | 0.169 | 0.010603 | 0.404 | 0.521 | 0.01502 | 0.173 | 0.243 | 0.127459 |
| Diacetylspermine | SPERMINE | | | | | | | | | | | |

Saliva for LC-MS is treated as follows.

1) In 270 μL of methanol and ammonium hydroxide solution adjusted to 2 μM 2-morpholinoethanesulfonic acid, saliva stored at −80° C. is dissolved, and 30 μL thereof is added and stirred.

2) The mixture is centrifuged at 4° C. and 15,000 rpm for 10 minutes, and the entire upper layer is transferred to another tube.

3) The whole amount of the liquid is subjected to centrifugal concentration, and added to the liquid are 18 μL of 90% MeOH and 12 μL of BorateBuffer, resulting in redissolution.

4) 5 μL of the liquid is used for LC-MS analysis, and 20 μL of the liquid is used for ELISA analysis.

5) In the LC-MS analysis, 10 μL of ultrapure water containing 4 μM Methionine-sulfone is added to 5 μL of the aforementioned solution to obtain a dilution as a sample.

Measurement conditions of LC-MS are as follows.

LC system: Agilent Technologies 1290 infinity
Mobile phase: Solvent A; Water containing 1% Formic acid: Solvent B; Acetonitrile
containing 0.1% formic acid
Flow rate: 0.5 mL/min

### Gradient [min. (% B)]: 0(98)-1(98)-3(55)-5(5)

Stop time: 7 min
Post time: 3 min
Column: CAPCELL CORE PC (Shiseido: 2.1 mm×50 mm, 2.7 mm)
Column temp.: 50° C.
Injection volume: 1 μL

### MS: Agilent Technologies G6230A

Gas temp: 350° C.
Gas flow: 13 L/min
Neblizer Gas: 55 psig

### Fragmentor: 150

### Skimmer: 90

### OCT1 RF Vpp: 200

### VCap: 3500

### Reference: 121.050873, 922.009798

### Mode: Positive

According to the present invention, when the concentration of saliva is corrected (normalized), using data analysis of a correlation network reduces the influence of the concentration. Even in saliva in which concentrations vary greatly, a subject with pancreatic cancer can be distinguished from a healthy subject. The present method makes prediction of chronic pancreatitis, IPMN, breast cancer, and oral cancer possible.

A range in which a test can be performed using the marker of the present invention is determined by the value of concentration-correcting marker that reflects the saliva concentration, and saliva whose overall concentration is outside should be treated as outliers. In saliva within the range, a patient with each cancer can be distinguished from a healthy subject by a mathematical model that combines the markers of absolute concentrations or corrected relative concentrations.

### INDUSTRIAL APPLICABILITY

Even by using saliva in which the concentration largely varies, pancreatic cancer, breast cancer, and oral cancer can be early detected in a healthy subject.

1-15. (canceled)

16. A method for assaying a salivary biomarker for pancreatic cancer, comprising the steps of:
collecting a saliva sample; and
detecting in the collected saliva sample whether a salivary biomarker for pancreatic cancer is present selected from the group consisting of N-acetylputrescine (N-Acetylputrescine), adenosine (Adenosine), 3-phospho-D-glyceric acid (3PG), urea (Urea), o-acetylcarnitine (o-Acetylcarnitine), citric acid (Citrate), glycyl-glycine (Gly-Gly), 5-aminovaleric acid (5-Aminovalerate), methyl 2-oxopentanoate (2-Oxoisopentanoate), malic acid (Malate), benzoate ester (Benzoate), fumaric acid (Fumarate), N-acetylaspartic acid (N-Acetylaspartate), inosine (Inosine), 3-methylhistidine (3-Methylhistidine), N1-acetylspermine (N1-Acetylspermine), creatine (Creatine), α-aminoadipic acid (alpha-Aminoadipate), phosphorylcholine (Phosphorylcholine), 2-hydroxypentanoate (2-Hydroxypentanoate), xanthine (Xanthine), succinic acid (Succinate), 6-phosphogluconic acid (6-Phosphogluconate), butanoic acid (Butanoate), homovanillic acid (Homovanillate), 0-phosphoserine (O-Phosphoserine), trimethylamine-N-oxide (Trimethylamine N-oxide), piperidine (Piperidine), cystine (Cystine), 2-isopropylmalic acid (2-Isopropylmalate), N8-acetylspermidine (N8-Acetylspermidine), N1-acetylspermidine (N1-Acetylspermidine), N-acetylneuraminic acid (N-Acetylneuraminate), glucosamine (Glucosamine), spermine (Spermine), agmatine (Agmatine), N-acetylhistamine (N-Acetylhistamine), methionine (Met), p-4-hydroxyphenylacetic acid (p-4-Hydroxyphenylacetate), N,N-dimethylglycine (N,N-Dimethylglycine), hypotaurine (Hypotaurine), glutamyl-glutamic acid (Glu-Glu), and N1,N12-diacetylspermine (N1,N12-Diacetylspermine), and combinations thereof.

17. The method according to claim 16, wherein the salivary biomarker for pancreatic cancer is selected from the group consisting of N8-acetylspermidine (N8-Acetylspermidine), creatinine (Creatinine), spermine (Spermine), aspartic acid (Asp), N1-acetylspermidine (N1-Acetylspermidine), N1-acetylspermine (N1-Acetylspermine), cytidine (Cytidine), α-aminoadipic acid (alpha-Aminoadipate), cytosine (Cytosine), betaine (Betaine), urea (Urea), homovanillic acid (Homovanillate), N-acetylneuraminic acid (N-Acetylneuraminate), cystine (Cystine), urocanic acid (Urocanate), fumaric acid (Fumarate), 1,3-diaminopropane (1,3-Diaminopropane), hypotaurine (Hypotaurine), nicotinic acid (Nicotinate), agmatine (Agmatine), valine (Val), 2-hydroxy-4-methylpentanoic acid (2-Hydroxy-4-methylpentanoate), alanine-alanine (Ala-Ala), citric acid (Citrate), glucosamine (Glucosamine), carnosine (Carnosine), glycyl-glycine (Gly-Gly), 2-aminobutyric acid (2AB), arginine (Arg), N-acylglutamic acid (N-Acetylglutamate), glycerophosphoric acid (Glycerophosphate), phosphoenolpyruvic acid (PEP), isoleucine (Ile), adenosine (Adenosine), guanine (Guanine), dihydroxyacetonephosphoric acid (DHAP), cadaverine (Cadaverine), and combinations thereof.

18. The method according to claim 16, wherein the salivary biomarker for pancreatic cancer is a combination of creatinine, N1-acetylspermidine, α-aminoadipic acid, N-acetylneuraminic acid, and 1,3-diaminopropane.

19. A method for identifying a patient with pancreatic cancer from a healthy person, comprising steps of:
collecting a saliva sample from the patient,
detecting in the collected saliva sample whether a salivary biomarker for pancreatic cancer is present, and
normalizing the concentration of the detected salivary biomarker using alanine.

20. The method according to claim 19, wherein the salivary biomarker for pancreatic cancer is selected from the group consisting of N-acetylputrescine (N-Acetylputrescine), adenosine (Adenosine), 3-phospho-D-glyceric acid (3PG), urea (Urea), o-acetylcarnitine (o-Acetylcarnitine), citric acid (Citrate), glycyl-glycine (Gly-Gly), 5-aminovaleric acid (5-Aminovalerate), methyl 2-oxopentanoate (2-Oxoisopentanoate), malic acid (Malate), benzoate ester (Benzoate), fumaric acid (Fumarate), N-acetylaspartic acid (N-Acetylaspartate), inosine (Inosine), 3-methylhistidine (3-Methylhistidine), N1-acetylspermine (N1-Acetylspermine), creatine (Creatine), α-aminoadipic acid (alpha-Aminoadipate), phosphorylcholine (Phosphorylcholine), 2-hydroxypentanoate (2-Hydroxypentanoate), xanthine (Xanthine), succinic acid (Succinate), 6-phosphogluconic acid (6-Phosphogluconate), butanoic acid (Butanoate), homovanillic acid (Homovanillate), O-phosphoserine (O-Phosphoserine), trimethylamine-N-oxide (Trimethylamine N-oxide), piperidine (Piperidine), cystine (Cystine), 2-isopropylmalic acid (2-Isopropylmalate), N8-acetylspermidine (N8-Acetylspermidine), N1-acetylspermidine (N1-Acetylspermidine), N-acetylneuraminic acid (N-Acetylneuraminate), glucosamine (Glucosamine), spermine (Spermine), agmatine (Agmatine), N-acetylhistamine (N-Acetylhistamine), methionine (Met), p-4-hydroxyphenylacetic acid (p-4-Hydroxyphenylacetate), N,N-dimethylglycine (N,N-Dimethylglycine), hypotaurine (Hypotaurine), glutamyl-glutamic acid (Glu-Glu), and N1,N12-diacetylspermine (N1,N12-Diacetylspermine), and combinations thereof.

21. The method according to claim 19, wherein the salivary biomarker for pancreatic cancer is selected from the group consisting of N8-acetylspermidine (N8-Acetylspermidine), creatinine (Creatinine), spermine (Spermine), aspartic acid (Asp), N1-acetylspermidine (N1-Acetylspermidine), N1-acetylspermine (N1-Acetylspermine), cytidine (Cytidine), α-aminoadipic acid (alpha-Aminoadipate), cytosine (Cytosine), betaine (Betaine), urea (Urea), homovanillic acid (Homovanillate), N-acetylneuraminic acid (N-Acetylneuraminate), cystine (Cystine), urocanic acid (Urocanate), fumaric acid (Fumarate), 1,3-diaminopropane (1,3-Diaminopropane), hypotaurine (Hypotaurine), nicotinic acid (Nicotinate), agmatine (Agmatine), valine (Val), 2-hydroxy-4-methylpentanoic acid (2-Hydroxy-4-methylpentanoate), alanine-alanine (Ala-Ala), citric acid (Citrate), glucosamine (Glucosamine), carnosine (Carnosine), glycyl-glycine (Gly-Gly), 2-aminobutyric acid (2AB), arginine (Arg), N-acylglutamic acid (N-Acetylglutamate), glycerophosphoric acid (Glycerophosphate), phosphoenolpyruvic acid (PEP), isoleucine (Ile), adenosine (Adenosine), guanine (Guanine), dihydroxyacetonephosphoric acid (DHAP), cadaverine (Cadaverine), and combinations thereof.

22. The method according to claim 19, wherein the salivary biomarker for pancreatic cancer is a combination of creatinine, N1-acetylspermidine, α-aminoadipic acid, N-acetylneuraminic acid, and 1,3-diaminopropane.
